SEQUENCE LISTING (33 sequences)

SEQ ID NO: 1 - DNA, 429 bp, Homo sapiens
  source 1..429: mol_type = genomic DNA
atgaggttctttgtccctctgttcctggtgggcatcctgttccctgccatcctggccaagcaattcacaaaatgtgagctgtcccagctgctgaaagacatagatggttatggaggcatcgctttgcctgaattgatctgtaccatgtttcacaccagtggttatgacacacaagccatagttgaaaacaatgaaagcacggaatatggactcttccagatcagtaataagctttggtgcaagagcagccaggtccctcagtcaaggaacatctgtgacatctcctgtgacaagttcctggatgatgacattactgatgacataatgtgtgccaagaagatcctggatattaaaggaattgactactggttggcccataaagccctctgcactgagaagctggaacagtggctttgtgagaagttgtga

SEQ ID NO: 2 - DNA, 1197 bp, Homo sapiens
  source 1..1197: mol_type = genomic DNA
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctag

SEQ ID NO: 3 - DNA, 1479 bp, Homo sapiens
  source 1..1479: mol_type = genomic DNA
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtga

SEQ ID NO: 4 - DNA, 1350 bp, Homo sapiens
  source 1..1350: mol_type = genomic DNA
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttc

SEQ ID NO: 5 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 6 - DNA, 587 bp, synthetic construct
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 7 - DNA, 1122 bp, synthetic construct
  source 1..1122: mol_type = other DNA
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatctgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 8 - DNA, 375 bp, synthetic construct
  source 1..375: mol_type = other DNA
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 9 - DNA, 1014 bp, Homo sapiens
  source 1..1014: mol_type = genomic DNA
atggctgccccgagagacaatgtcactttattattcaagttatactgcttggcagtgatgaccctgatggctgcagtctataccatagctttaagatacacaaggacatcagacaaagaactctacttttcaaccacagccgtgtgtatcacagaagttataaagttattgctaagtgtgggaattttagctaaagaaactggtagtctgggtagattcaaagcatctttaagagaaaatgtcttggggagccccaaggaactgttgaagttaagtgtgccatcgttagtgtatgctgttcagaacaacatggctttcctagctcttagcaatctggatgcagcagtgtaccaggtgacctaccagttgaagattccgtgtactgctttatgcactgttttaatgttaaaccggacactcagcaaattacagtgggtttcagtttttatgctgtgtgctggagttacgcttgtacagtggaaaccagcccaagctacaaaagtggtggtggaacaaaatccattattagggtttggcgctatagctattgctgtattgtgctcaggatttgcaggagtatattttgaaaaagttttaaagagttcagatacttctctttgggtgagaaacattcaaatgtatctatcagggattattgtgacattagctggcgtctacttgtcagatggagctgaaattaaagaaaaaggatttttctatggttacacatattatgtctggtttgtcatctttcttgcaagtgttggtggcctctacacttctgttgtggttaagtacacagacaacatcatgaaaggcttttctgcagcagcggccattgtcctttccaccattgcttcagtaatgctgtttggattacagataacactcacctttgccctgggtactcttcttgtatgtgtttccatatatctctatggattacccagacaagacactacatccatccaacaaggagaaacagcttcaaaggagagagttattggtgtgtga

SEQ ID NO: 10 - DNA, 24 bp, synthetic construct
  source 1..24: mol_type = other DNA
ctgctgaccaaggtgaagggctcc

SEQ ID NO: 11 - DNA, 66 bp, synthetic construct
  source 1..66: mol_type = other DNA
cccagacaagacactacatccatccaacaaggagaaacagcttcaaaggagagagttattggtgtg

SEQ ID NO: 12 - DNA, 3935 bp, synthetic construct
  source 1..3935: mol_type = other DNA
atgaggttctttgtccctctgttcctggtgggcatcctgttccctgccatcctggccaagcaattcacaaaatgtgagctgtcccagctgctgaaagacatagatggttatggaggcatcgctttgcctgaattgatctgtaccatgtttcacaccagtggttatgacacacaagccatagttgaaaacaatgaaagcacggaatatggactcttccagatcagtaataagctttggtgcaagagcagccaggtccctcagtcaaggaacatctgtgacatctcctgtgacaagttcctggatgatgacattactgatgacataatgtgtgccaagaagatcctggatattaaaggaattgactactggttggcccataaagccctctgcactgagaagctggaacagtggctttgtgagaagttgtgaggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagctcgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatctgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 13 - DNA, 3163 bp, synthetic construct
  source 1..3163: mol_type = other DNA
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggcgcgcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 14 - DNA, 3932 bp, synthetic construct
  source 1..3932: mol_type = other DNA
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagggatcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgaggttctttgtccctctgttcctggtgggcatcctgttccctgccatcctggccaagcaattcacaaaatgtgagctgtcccagctgctgaaagacatagatggttatggaggcatcgctttgcctgaattgatctgtaccatgtttcacaccagtggttatgacacacaagccatagttgaaaacaatgaaagcacggaatatggactcttccagatcagtaataagctttggtgcaagagcagccaggtccctcagtcaaggaacatctgtgacatctcctgtgacaagttcctggatgatgacattactgatgacataatgtgtgccaagaagatcctggatattaaaggaattgactactggttggcccataaagccctctgcactgagaagctggaacagtggctttgtgagaagttgtgactcgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatctgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 15 - DNA, 962 bp, synthetic construct
  source 1..962: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 16 - DNA, 3935 bp, synthetic construct; identical to SEQ ID NO: 12
  source 1..3935: mol_type = other DNA

SEQ ID NO: 17 - DNA, 3187 bp, synthetic construct
  source 1..3187: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgctgctgaccaaggtgaagggctcctgaggcgcgcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 18 - DNA, 3935 bp, synthetic construct; identical to SEQ ID NO: 12
  source 1..3935: mol_type = other DNA

SEQ ID NO: 19 - DNA, 3163 bp, synthetic construct
  source 1..3163: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgaggcgcgcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 20 - DNA, 2144 bp, synthetic construct
  source 1..2144: mol_type = other DNA
atgaggttctttgtccctctgttcctggtgggcatcctgttccctgccatcctggccaagcaattcacaaaatgtgagctgtcccagctgctgaaagacatagatggttatggaggcatcgctttgcctgaattgatctgtaccatgtttcacaccagtggttatgacacacaagccatagttgaaaacaatgaaagcacggaatatggactcttccagatcagtaataagctttggtgcaagagcagccaggtccctcagtcaaggaacatctgtgacatctcctgtgacaagttcctggatgatgacattactgatgacataatgtgtgccaagaagatcctggatattaaaggaattgactactggttggcccataaagccctctgcactgagaagctggaacagtggctttgtgagaagttgtgactcgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatctgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 21 - DNA, 962 bp, synthetic construct; identical to SEQ ID NO: 15
  source 1..962: mol_type = other DNA

SEQ ID NO: 22 - DNA, 2912 bp, synthetic construct
  source 1..2912: mol_type = other DNA
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagctcgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatctgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 23 - DNA, 962 bp, synthetic construct; identical to SEQ ID NO: 15
  source 1..962: mol_type = other DNA

SEQ ID NO: 24 - DNA, 3935 bp, synthetic construct; identical to SEQ ID NO: 12
  source 1..3935: mol_type = other DNA

SEQ ID NO: 25 - DNA, 3037 bp, synthetic construct
  source 1..3037: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttctgaggcgcgcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 26 - DNA, 3935 bp, synthetic construct; identical to SEQ ID NO: 12
  source 1..3935: mol_type = other DNA

SEQ ID NO: 27 - DNA, 3103 bp, synthetic construct
  source 1..3103: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttccccagacaagacactacatccatccaacaaggagaaacagcttcaaaggagagagttattggtgtgtgaggcgcgcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 28 - DNA, 3935 bp, synthetic construct; identical to SEQ ID NO: 12
  source 1..3935: mol_type = other DNA

SEQ ID NO: 29 - DNA, 3229 bp, synthetic construct
  source 1..3229: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgcccagacaagacactacatccatccaacaaggagaaacagcttcaaaggagagagttattggtgtgtgaggcgcgcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 30 - protein, 8 aa, synthetic construct
LLTKVKGS

SEQ ID NO: 31 - protein, 22 aa, synthetic construct
PRQDTTSIQQGETASKERVIGV

SEQ ID NO: 32 - protein, 18 aa, synthetic construct
TTSIQQGETASKERVIGV

SEQ ID NO: 33 - DNA, 54 bp, synthetic construct
  source 1..54: mol_type = other DNA
actacatccatccaacaaggagaaacagcttcaaaggagagagttattggtgtg

(19) United States
(12) Patent Application Publication
(10) Pub. No.: US 2025/0011825 A1
(43) Pub. Date: Jan. 9, 2025

(54) Lactose And Human Milk Oligosaccharides (HMOS) Production In Cells

(71) Applicant: AGENCY FOR SCIENCE, TECHNOLOGY AND RESEARCH, Singapore (SG)

(72) Inventors: Zhiwei Song, Singapore (SG); Wen Min Lau, Singapore (SG)

(21) Appl. No.: 18/711,011

(22) PCT Filed: Dec. 20, 2022
(86) PCT No.: PCT/SG2022/050919

§ 371 Date: May 16, 2024

(30) Foreign Application Priority Data
Dec. 20, 2021 (SG) 10202114129T

(51) Int. Cl. C12P 19/18; C07K 14/76; C12N 9/10; C12N 15/52; C12P 19/12

(52) U.S. Cl. CPC C12P 19/18; C07K 14/76; C12N 9/1051; C12N 15/52; C12P 19/12; C12Y 204/01038; C12N 2510/02

(57) ABSTRACT

This technology relates to the production of lactose and human milk oligosaccharides (HMOs) in cells.

### CROSS-REFERENCE TO RELATED APPLICATIONS

This application claims the benefit of priority of Singapore patent application Ser. No. 10202114129T, filed 20 Dec. 2021, the contents of it being hereby incorporated by reference in its entirety for all purposes.

### FIELD OF THE INVENTION

The present invention relates to the field of molecular biology and cell biology. In particular, the present invention relates to the production of lactose and human milk oligosaccharides (HMOs) in cells.

### BACKGROUND

Human milk and cow milk contain similar amounts of lactose which is the major energy source for new-borns. However, human milk contains high concentrations of oligosaccharides that are missing in cow milk and other milk obtained from farmed animals. More than 130 types of human milk oligosaccharides (HMOs) have been described with about 15 of them being highly abundant in human milk. Studies have shown that human milk oligosaccharides (HMOs) can modulate the infant's microbiota, immune system and brain development. It has been observed that breastfed babies have a much higher chance of survival and lower incidences of disease compared to bottle-fed babies.

To improve the nutritional profile of baby milk powder, human milk oligosaccharides (HMOs) are commonly added to milk powder during manufacture. For example, companies add 2′-fucosyllactose (2′-FL) to baby milk powder which is the most abundant human milk oligosaccharides (HMOs) found in human milk. However, the development and production of other abundant human milk oligosaccharides (HMOs) remains unsuccessful.

Conventionally, 2′-FL that was added to baby milk powder is produced in microorganisms such as fungi and bacteria. It is challenging to produce other human milk oligosaccharides (HMOs) in fungi, in particular for those with higher structural complexity. Therefore, an object of the present invention is the production of human milk oligosaccharides (HMOs) using expression systems such as mammalian cells.

### SUMMARY OF INVENTION

In one aspect, the present disclosure refers to a recombinant cell for producing lactose, wherein said recombinant cell comprises one or more expression construct that encodes an alpha-lactalbumin (LALBA) and a beta-1,4-galactosyltransferase 1 (B4GalT1).

In another aspect, the present disclosure refers to a composition comprising one or more expression construct, wherein the expression construct comprises polynucleotides encoding an alpha-lactalbumin (LALBA) and a beta-1,4-galactosyltransferase 1 (B4GalT1).

In another aspect, the present disclosure refers to a method of producing lactose using the recombinant cell as disclosed herein, wherein the method comprises the steps of: i) culturing the recombinant cell of any one of the preceding claims, and ii) detecting lactose from the recombinant cell in i).

In another aspect, the present disclosure refers to a cell culture comprising the cell as disclosed herein and a culture medium.

In yet another aspect, the present disclosure refers to a cryopreserved cell culture comprising the cell as disclosed herein.

### BRIEF DESCRIPTION OF THE DRAWINGS

FIG. 1 shows the different subcellular localizations of GFP and Glucose Transporter 1 (GLUT1) fusion proteins in mammalian cells. Lactose synthesis takes place in the Golgi of the mammary gland epithelial cells with the functions of lactose synthase (LS) heterodimer α-Lactalbumin (LALBA) and β-1,4-galactosyltransferase (B4GalT1). One of starting materials for lactose synthesis, glucose, is transported by glucose transporters in cells. However, glucose transporter 1 (GLUT1) is normally expressed on the cell surface. Thus, GLUT1 variants were generated to identify a suitable variant that remains in the ER or Golgi apparatus, instead of being translocated to the cell membrane FIG. 1A and FIG. 1C show localization of Golgi in Chinese Hamster Ovary (CHO) cells grown in suspension culture. Fluorescent microscopic image of GFP-GLUT1 (GFP fused at the N-terminus of huGLUT1) as shown in FIG. 1B is mostly localized to the cell membrane whereas GLUT1-GFP (GFP fused at the C-terminus of huGLUT1) in FIG. 1D is only partially localized to the cell membrane in CHO cells grown in suspension culture.

FIG. 2 provides fluorescent confocal microscopic images showing cellular localizations of additional variants of the GLUT1/GFP fusion proteins. The tested variants comprise: LGCT: a full-length human GLUT1 with a C-terminal fusion of GFP; LGNT: a full-length human GLUT1 with an N-terminal fusion of GFP; ST2: a full-length human GLUT1 with an N-terminal fusion of GFP and an ER retention signal (obtained from human Glucose Transporter 2, having the sequence of LLTKVKGS (SEQ ID NO: 30)); ST3: a full-length human GLUT1 with an N-terminal fusion of GFP and a Golgi retention signal (obtained from the C-terminal 18 amino acids of human CMP-sialic acid transporter (CST), having the sequence of TTSIQQGETASKERVIGV (SEQ ID NO: 32); ST5: a truncated human GLUT1 with an N-terminal fusion of GFP; ST7: a full-length human GLUT1 with an N-terminal fusion of GFP and a C-terminal ER retention signal; ST4: a full-length human GLUT1 with an N-terminal fusion of GFP and a C-terminal Golgi retention signal; and ST6: a truncated human GLUT1 with an N-terminal fusion of GFP and a C-terminal Golgi retention signal. According to FIG. 2, the exemplary variants show various degree of co-localisation between the GLUT1/GFP fusion proteins and the Golgi marker to carry out the desired function of transporting glucose into the ER and Golgi apparatus. In particular, ST7, ST5, and ST6 show high co-localisation between GLUT1/GFP fusion proteins and the Golgi marker, indicating retention of GLUT1/GFP fusion protein in the Golgi apparatus.

FIG. 3 provides Liquid Chromatography-Mass Spectrometry (LC-MS) results showing lactose synthesis in cultured recombinant cells. The recombinant cells express α-Lactalbumin (LALBA), β-1,4-galactosyltransferase (B4GalT1), or glucose transporter GLUT1, or combinations thereof. Conditioned media culturing recombinant cells is analysed with Liquid Chromatography-Mass Spectrometry against a lactose standard control. The top panel shows the standard peak for lactose when analysed using the same LC-MS procedure. Cells expressing alpha-lactalbumin (LALBA) or galactosyltransferase (B4GalT1) alone do not produce detectable lactose. As shown in the two panels at the bottom, the same lactose peak can be detected in the cultured Chinese Hamster Ovary (CHO) cells that were transfected with B4GalT1+LALAB+GFP-GLUT1 (LGNT), or B4GalT1+LALBA constructs. Both B4GalT1 and LALBA are required for lactose synthesis in mammalian cells. Interestingly, sucrose was also produced in CHO cells (the peak on the left side of the lactose peak).

FIG. 4 confirms the identity of the synthesized product using the Liquid Chromatography-Mass Spectrometry (LC-MS) following the identification of lactose produced by the recombinant cells as described herein. The conditioned medium that contained lactose after culturing the CHO cells expressing the lactose synthesis constructs was treated with β-galactosidase, which specifically digests lactose. As shown in the top panel of FIG. 4, after the treatment, the lactose peak disappeared, whereas the sucrose peak remained unchanged. The disappearance of the lactose peak in LC-MS graph after treatment of lactose digesting enzyme confirms the synthesis of lactose by recombinant cells.

FIG. 5 provides a quantitative comparison of lactose product by different combinations of constructs. The schematics of FIG. 5A provide an overview of the constructs and the combination of constructs for each transfection tested, including combinations including LALBA and B4GalT1, with or without GLUT1 variants. The quantitative results of lactose production in each stably transfected pool are shown in FIG. 5B. The concentrations of lactose were determined by the K-LOLAC lactose assay kit. As can be seen from FIG. 5B, all of these constructs are capable of producing Lactose, compared to the negative controls (LALBA or B4GalT1 alone). CT+GL constructs (C-terminus tagged full-length huGLUT1, LALBA and B4GalT1) produced the highest amount of lactose compared to other tested combinations, followed by ST7+GL.

FIG. 6 shows the identification of the putative human milk oligosaccharides (HMOs) in recombinant cells expressing huGLUT1, B4GalT1 and LALBA. Hitchhiking the internal cellular mechanism, the recombinant cells are able to convert the produced lactose into oligosaccharides. Monosaccharides and polysaccharides present in the conditioned media were captured and purified with BlotGlyco Kit, labeled with 2-AB, were analyzed with a liquid chromatography quadrupole time-of-flight mass spectrometry (LC-FLD-QTOF). Two samples were analysed, the Control (C1) medium (untransfected CHO cells), and the Sample(S) medium (CHO cells transfected with huGLUT1, B4GalT1 and LALBA). Three different amounts of Sample S were analysed, S1 (20 ul), S2 (80 μl) and S3 (160 μl). In FIG. 6A, similar amounts of glucose were detected in both the Control and the Sample (C and S1-3). Lactose was detected only in the Sample, not in the Control in FIG. 6B. Higher amounts of lactose were detected when more sample were used in the analysis. According to FIG. 6C, Sialyl-lactose was detected in the Sample, but not in the Control. Similarly, FIG. 6D-FIG. 6H, show that five other different putative HMOs were detected in the Sample, not in the Control. Based on FIG. 6H, apart from the commonly found milk oligosaccharides identified above, other milk oligosaccharides are produced as well (arrows). A summary of the identified polysaccharides produced by CHO cells transfected with huGLUT1, B4GalT1 and LALBA is provided in FIG. 6I. The identities of the products in FIGS. 6A-6H respectively are: 6A: glucose; 6B: lactose; 6C: sialyl-lactose; 6D: putative Lacto-n-neotatraose (LNnT) oligosaccharide (4 sugars); 6E: putative sialyl-LNnT (4 sugars+sialic acid); 6F: putative para-lacto-N-hexaose (Para-LNH) (6 sugars); 6G: putative sialyl-para-LNH (6 sugars+sialic acid); and 6H: putative para-lacto-N-octaose (8 sugars). Hence, the recombinant cells comprising the expression constructs as described herein are able to produce human milk oligosaccharides.

FIG. 7 shows the further confirmation of putative HMOs produced by a CHO-K-ST7 stable cell line. FIG. 7A provides a Liquid Chromatography Fluorescence Detector (LC-FLD) chromatogram showing a peak at 3.6 min which was putatively identified as 2-AB labelled sialyllactose, based on known calculated retention times of HMOs. A further detailed fragmentation pattern of the oligosaccharide as shown in FIG. 7B confirms that the putative structure is sialyllactose (3′-Sialyllactose (3′SL) or 6′-Sialyllactose (6′SL)).

FIG. 7C provides a LC-FLD chromatogram having a peak at 3.8 min which was putatively identified as 2-AB labelled Lacto-n-neotatraose (LNnT), based on known calculated retention times of HMOs. In addition, FIG. 7D provides a further detailed fragmentation pattern of the oligosaccharide LNnT showing individual monosaccharides. These experiments confirmed the presence of known human milk oligosaccharides (HMOs) produced by recombinant CHO cells.

FIG. 8 provides exemplary schematics showing the expression constructs used in the Examples to produce lactose and HMOs, and the controls used, respectively.

FIG. 8A-8I each comprise the vector maps of a single mammalian expression vector showing the individual components and their relative positioning within the vector. The respective vectors are, from FIG. 8A-8I, respectively: 1. hLALBA-B4GALT1-hGLUT1-GFP (CT); 2. hB4GALT1-hLALBA; 3. hLALBA-B4GALT1-GFP-hGLUT1-ER (ST7); 4. hLALBA-B4GALT1-GFP-hGLUT1 (NT/LGNT); 5. hLALBA (negative control); 6. B4GALT1 (negative control); 7. hLALBA-B4GALT1-GFP-hGLUT1A (ST5); 8. hLALBA-B4GALT1-GFP-hGLUT1A-golgi (ST6); 9. hLALBA-B4GALT1-GFP-hGLUT1A-golgi (ST4).

FIG. 9 provides exemplary nucleic acid sequence of the expressed proteins used in the Examples to produce lactose and HMOs, including human LALBA, human B4GalT1, and human GLUT1.

### DEFINITIONS

As used herein, the term “recombinant cell” refers to a cell that that is made by combining genetic material from two or more different sources. Recombination is a process by which pieces of genetic material are broken and recombined to produce new combinations of alleles. The process of recombination can happen both naturally or engineered artificially in the laboratory. One example of naturally occurring recombination is meiosis, where the homologous pairs of maternal and paternal chromosomes align and crossover, causing exchange of genetic material between the maternal and paternal chromosomes. As a result, offspring can have different combinations of genes than their parents. The genetic engineering of cells in the laboratory allows manipulation of genetic material such as DNA. In particular, genetic manipulation introduces exogenous genetic material into the host cell, thereby altering the characteristics of the host cell. Methods of genetic manipulation are known in the art, for example, virus transfection, electroporation, or microinjection. Recombinant cells as referred to herein can be, but are not limited to mammalian cells, fungi cells, insect cells, plant cells or bacterial cells.

As used herein, the term “lactose” refers to a disaccharide sugar synthesized by one galactose and one glucose subunit which form a β-1→4 glycosidic linkage. Lactose makes up around 2-8% of milk (by mass).

As used herein, the term “milk oligosaccharides” refers to unconjugated glycan/carbohydrates, which are found primarily in breast milk. Oligosaccharides are the third most abundant component in human milk. It is widely accepted that they play several important protective, physiological, and biological roles, including selective growth stimulation of beneficial gut microbiota, inhibition of pathogen adhesion, and immune modulation.

As used herein, the term “human milk oligosaccharides (HMOs)” refers to milk oligosaccharides that are isolated or obtained from human breast milk. Human milk oligosaccharides (HMOs) are made of linear or branched monosaccharides, such as galactose, glucose, N-acetylglucosamine, fucose, and sialic acid, varying in size from 3 to 22 monosaccharide units. In contrast to the milk of other mammals, human breast milk contains a very high amount and a structurally diverse set of oligosaccharides that even exceeds the protein content of breast milk. Commonly found human milk oligosaccharides (HMOs) include, for example, 2′-Fucosyllactose (2′-FL), 3′-Fucosyllactose (3′-FL), Lacto-N-tetraose (LNT), Lacto-N-neotetraose (LNnT), Lacto-N-fucopentaose I (LNFPI), Lacto-N-fucopentaose II (LNFPII), Lacto-N-fucopentaose III (LNFPIII), 3′-Sialyllactose (3′-SL), 6′-Sialyllactose (6′-SL), Sialyllacto-N-tetraose (a) (LSTa), Sialyllacto-N-tetraose (b) (LSTb), Sialyllacto-N-tetraose (c) (LSTc), 6′-Sialyllactosamine (6′ SLN), Disialyllactose (DSL), Disialyllactose-N-tetraose (DSLNT), α-3′-Galactosyllactose (α3′-GL), β-3′-Galactosyllactose (β3′-GL), β-4′-Galactosyllactose (4′-GL), 3-6′-Galactosyllactose (6′-GL), α-3′-N-acetylgalactosaminyllactose (α-3′-GalNACL), Lacto-N-difucohexaose I (LNDFH-I), Lacto-N-neohexaose (LNnH), Lacto-N-hexaose (LNH), and 6′-N-Acetyl-glucosaminyl-lactose (NAL).

As used herein, the term “expression construct” or “expression vector” refers to a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. An expression vector has features that any vector may have, such as an origin of replication, a selectable marker, and a suitable site for the insertion of a gene like the multiple cloning site. The vector is typically engineered to contain elements necessary for gene expression. Such elements may include a promoter, the correct translation initiation sequence such as a ribosomal binding site and start codon, a termination codon, and a transcription termination sequence. There are differences in the machinery for protein synthesis between prokaryotes and eukaryotes, therefore the expression vectors must have the elements for expression that are appropriate for the chosen host. The expression vector is transformed or transfected into the host cell for protein synthesis. Thus, some expression vectors may have elements for transformation or the insertion of DNA into the host chromosome, for example the vir genes for plant transformation, and integrase sites for chromosomal integration.

As used herein, the term “alpha-Lactalbumin (LALBA)” or “α-Lactalbumin (LALBA)” refers to a protein encoded by the LALBA gene. α-Lactalbumin is a protein that regulates the production of lactose in the milk of almost all mammalian species. In primates, alpha-lactalbumin expression is upregulated in response to the hormone prolactin and increases the production of lactose. α-Lactalbumin forms the regulatory subunit of the lactose synthase (LS) heterodimer while β-1,4-galactosyltransferase (B4GalT1) forms the catalytic component. Together, these proteins enable LS to produce lactose by transferring galactose moieties to glucose.

As used herein, the term “beta-1,4-galactosyltransferase 1 (B4GalT1)” or “β-1,4-galactosyltransferase (B4GalT1)” refers to a type II membrane-bound glycoprotein that appear to have exclusive specificity for the donor substrate UDP-galactose. The glycoprotein transfers galactose in a β-1,4-linkage to similar acceptor sugars, such as GlcNAc, Glucose, and Xylose.

As used herein, the term “glucose transporter (GLUT)” refers to a wide group of membrane proteins that facilitate the transport of glucose across the plasma membrane, a process known as facilitated diffusion. The GLUT or SLC2A family are a protein family that is found in most mammalian cells. GLUTs are integral membrane protein that contains 12 membrane-spanning helices with both the amino and carboxyl termini exposed on the cytoplasmic side of the plasma membrane. GLUT proteins transport glucose and related hexoses according to a model of alternate conformation, which predicts that the transporter exposes a single substrate binding site toward either the outside or the inside of the cell. Binding of glucose to one site provokes a conformational change associated with transport, and releases glucose to the other side of the membrane. The inner and outer glucose-binding sites are predicted to be located in transmembrane segments 9, 10, 11 Also, the DLS motif located in the seventh transmembrane segment is potentially involved in the selection and affinity of transported substrates. Fourteen GLUTs are encoded by human genome, such as glucose transporter 1 (GLUT1), a glucose transporter 8 (GLUT8), a glucose transporter 12 (GLUT12), and a sodium-glucose transporter (SGLT1). Each glucose transporter isoform plays a specific role in glucose metabolism determined by its pattern of tissue expression, substrate specificity, transport kinetics, and regulated expression in different physiological conditions.

As used herein, the term “GLUT1” refers to glucose transporter 1, a well-characterised isoform of the GLUT protein family. GLUT1 is widely distributed in fetal tissues. In the adult, it is expressed at highest levels in erythrocytes and also in the endothelial cells of barrier tissues such as the blood-brain barrier. However, it is responsible for the low level of basal glucose uptake required to sustain respiration in all cells.

As used herein, the term “fluorescence protein” refers to proteins that are members of a structurally homologous class that share the unique property of being self-sufficient to form a visible wavelength chromophore from a sequence of 3 amino acids within their own polypeptide sequence. It is common research practice for biologists to introduce a gene (or a gene chimera) encoding an engineered fluorescent protein into living cells and subsequently visualize the location and dynamics of the gene product using fluorescence microscopy. Through extensive engineering, a wide range of fluorescence proteins are developed with various excitation and emission wavelengths, maturation rate, and sizes. Fluorescent proteins commonly used in research includes, for example, Green Fluorescent Proteins (GFP), Red Fluorescent Proteins (RFP), and Yellow Fluorescent Proteins (YFP).

As used herein, the term “Endoplasmic Reticulum (ER) localization sequence” or “Endoplasmic Reticulum (ER) retention sequence” refers to a sequence that allows a protein to localize within Endoplasmic Reticulum. Protein localization to the ER often depends on certain sequences of amino acids located at the N terminus or C terminus, which are known as signal peptides, molecular signatures, or sorting signals. The classical ER retention signal is the C-terminal KDEL sequence for lumen bound proteins and KKXX (signal sequence is located in cytoplasm) for transmembrane localization. These signals allow for retrieval from the Golgi apparatus by ER retention receptors, effectively maintaining the protein in the ER. For example, as used herein, the ER localization sequence is LLTKVKGS (SEQ ID NO: 30) (exemplary nucleic acid sequence:
CTGCTGACCAAGGTGAAGGGCTCC (SEQ ID NO: 10)). Other sequences can be used for ER retention of proteins are known in the art.

As used herein, the term “Golgi localization sequence” or “Golgi retention sequence” refers to a sequence that allows a protein to localize within the Golgi apparatus. For example, as used herein, the Golgi localization sequence is
PRQDTTSIQQGETASKERVIGV (SEQ ID NO: 31) (exemplary nucleic acid sequence can be:
CCCAGACAAGACACTACATCCATCCAACAAGGAGAAACAGCTTCAAAGGAGAGAGTTAT TGGTGTG (SEQ ID NO: 11), or TTSIQQGETASKERVIGV (SEQ ID NO: 32). Unlike ER retention of proteins, which shares a consensus signal sequence, protein retention in Golgi develops more dynamic and diverse mechanisms. Other sequences can be used for Golgi retention of proteins and are known in the art, such as the direct or indirect associations of protein's transmembrane domain or motif with a COPI-coatomer.

As used herein, the term “selectable marker” refers to genes that help identify host cells that have successfully transformed, or taken up the recombinant plasmid. Selectable marker genes are a vital part of most transformation protocols. They are delivered alongside the gene of interest, either on the same plasmid or on a separate plasmid. A wide range of selectable marker regimes is available and is particularly important in species where transformation efficiencies are low. Selectable marker genes can be categorized into those based on resistance genes that confer the ability to grow in the presence of toxic compounds such as antibiotics or herbicides which kill or otherwise compromise untransformed tissue (negative selection). Commonly used negative selection markers include antibiotic resistance gene marker in combination with antibiotic compounds, for example, kanamycin, ampicillin, or hygromycin. Alternatively, a range of positive selection systems are available which provide transformed tissues with an enhanced ability to utilize, e.g., an unusual carbohydrate or amino acid supply and thus enrich the culture for transformed tissue expressing the marker gene. For example, glutamine synthetase (GS) selection system, or dihydrofolate reductase (DHFR) selection system.

As used herein, the term “sequence identity” refers to the percentage of similarity between a pair of sequences. The sequence identity applies to either protein or peptide sequence, or polynucleotides. The sequence identity between two sequences can be, for example, 90%, 95%, 98%, 99% or 100%. The higher the percentage of similarity is, the more the two sequences have in common in their sequences. Two sequences are completely identical if the sequence identity is 100%.

### DETAILED DESCRIPTION OF THE PRESENT INVENTION

The following detailed description is merely exemplary in nature and is not intended to limit the invention or the application and uses of the invention. Furthermore, there is no intention to be bound by any theory presented in the preceding background of the invention or the following detailed description. It is an object of this invention to produce human milk oligosaccharides (HMOs) using mammalian system, and the expression vectors, polynucleotides, and recombinant cells for the production of HMOs thereof.

All HMOs are derivatives of lactose. Biosynthesis of lactose takes place in the Golgi apparatus of human mammary epithelial cells (MEC). The lactose synthase (LS) enzyme synthesizes lactose (galactose β-1,4-glucose) from UDP-galactose and glucose. Lactose synthesis takes place in the Golgi apparatus of mammary gland epithelial cells. The LS is an enzymatic complex of galactosyltransferase (B4GalT1) and alpha-lactalbumin (LALBA). LALBA is only found in mammary epithelial cells and can increase the affinity of B4GalT1 for glucose by 1000-fold. Therefore, LALBA enables B4GalT1 to add galactose from UDP-galactose to glucose even at low concentrations of glucose. In addition, glucose transporters can transport glucose into the Golgi apparatus, which increases the amount of glucose in the Golgi apparatus.

In one aspect, the disclosure provides a recombinant cell for producing lactose. In one embodiment, the recombinant cell is a mammalian cell. In one example, the recombinant cell is a stable cell line. In a further example, the recombinant cell is a Chinese Hamster Ovary (CHO) cell. In yet a further example, the recombinant cell is a CHO-K1 cell line. A person skilled in the art would appreciate that the recombinant cell is a cell that allows stable expression of recombinant proteins, for example, HeLa, HEK293T, U2OS, A549, HT1080, CAD, P19, NIH3T3, L929, N2a, MCF-7, Y79, SO-Rb50, Hep G2, DUKX-X11, J558L, or Baby hamster kidney (BHK) cells.

In one embodiment, the recombinant cell comprises one or more expression construct(s). It is understood by a person skilled in the art that the expression construct is for the purpose of expressing recombinant proteins. In one example, the expression construct is a plasmid vector. In another example, the expression constructs may comprise a vector backbone, one or more of any one of the following: origins of replication, a selection marker, a reporter (for example, a fluorescent protein), a promoter, an internal ribosome entry site (IRES), a linker sequence, and multiple cloning sites. The general setup of such expression constructs is known in the art. In another example, the expression construct is a mammalian expression construct. In another example, the expression construct comprises one or more promoters that can drive gene expression in mammalian cells. Commonly used vector for expression of recombinant protein is known in the art, and are usually commercially available, for example, pcDNA3.1, pGenLenti, and pCMV.

In one embodiment, the recombinant cell comprises one or more expression constructs that encode an alpha-lactalbumin (LALBA) and a beta-1,4-galactosyltransferase 1 (B4GalT1). In one example, the alpha-lactalbumin (LALBA) and a beta-1,4-galactosyltransferase 1 (B4GalT1) are encoded within the same construct. In another example, the alpha-lactalbumin (LALBA) and a beta-1,4-galactosyltransferase 1 (B4GalT1) are encoded in separate constructs. In another example, the alpha-lactalbumin (LALBA) and a beta-1,4-galactosyltransferase 1 (B4GalT1) encoded in the expression construct are of mammalian origin. In a further example, the alpha-lactalbumin (LALBA) and a beta-1,4-galactosyltransferase 1 (B4GalT1) encoded in the expression construct are of human origin. In a further example, the alpha-lactalbumin (LALBA) and a beta-1,4-galactosyltransferase 1 (B4GalT1) encoded in the expression construct are from a hamster. In yet another example, the alpha-lactalbumin (LALBA) and a beta-1,4-galactosyltransferase 1 (B4GalT1) encoded in the expression construct are from the same species. In yet another example, the alpha-lactalbumin (LALBA) and a beta-1,4-galactosyltransferase 1 (B4GalT1) encoded in the expression construct are from the same genus. In yet another example, the alpha-lactalbumin (LALBA) and a beta-1,4-galactosyltransferase 1 (B4GalT1) encoded in the expression construct are from different species. The alpha-lactalbumin (LALBA) and a beta-1,4-galactosyltransferase 1 (B4GalT1) encoded in the expression construct may be expressed under the control of the same promoter, or different promoters. The alpha-lactalbumin (LALBA) and a beta-1,4-galactosyltransferase 1 (B4GalT1) encoded in the expression construct may be tagged or untagged, by a reporter. In one example, the reporter is a fluorescent protein. In a further example, the fluorescent protein is a Green Fluorescent Protein (GFP).

In one embodiment, the disclosure provides a recombinant cell for producing lactose, wherein said recombinant cell comprises one or more expression constructs that encode an alpha-lactalbumin (LALBA) and a beta-1,4-galactosyltransferase 1 (B4GalT1). In one example, the alpha-lactalbumin (LALBA) encoded in the expression construct has a nucleic acid sequence of SEQ ID NO: 1. In another example, beta-1,4-galactosyltransferase 1 (B4GalT1) encoded in the expression construct has a nucleic acid sequence of SEQ ID NO: 2. In another example, the alpha-lactalbumin (LALBA) encoded in the expression construct has a nucleic acid sequence that is 90%, 95%, 96%, 97%, 98%, 99%, or 99.9% identical to SEQ ID NO: 1. In another example, the beta-1,4-galactosyltransferase 1 (B4GalT1) encoded in the expression construct has a nucleic acid sequence that is 90%, 95%, 96%, 97%, 98%, 99%, or 99.9% identical to SEQ ID NO: 2. Irrespective of the sequence identity percentage, the alpha-lactalbumin (LALBA) and the beta-1,4-galactosyltransferase 1 (B4GalT1) are capable of lactose synthesis.

In another embodiment, the disclosure provides a recombinant cell for producing lactose, wherein the recombinant cell further comprises a glucose transporter. In one example, the recombinant cell further comprises an expression construct encoding the glucose transporter. In another example, the glucose transporter is encoded in the same expression construct with one or more expression constructs that encode an alpha-lactalbumin (LALBA) or a beta-1,4-galactosyltransferase 1 (B4GalT1), or both. In one example, the sequences encoding alpha-lactalbumin (LALBA), beta-1,4-galactosyltransferase 1 (B4GalT1), glucose transporter, and combinations thereof, are engineered in at least one, at least two, at least three or at least four expression constructs. In one example, the glucose transporter is a mammalian glucose transporter. In another example, the glucose transporter is a human glucose transporter. In a further example, the glucose transporter is from GLUT family. In another example, the glucose transporter is selected from any one of the following: GLUT 1, GLUT2, GLUT3, GLUT4, GLUT5, GLUT6, GLUT7, GLUT8, GLUT9, GLUT10, GLUT11, GLUT12, HMIT (H+ driven myoinositol transporter, also GLUT13), GLUT14, and sodium-glucose transporter (SGLT1). In yet another example, the glucose transporter is GLUT1. In one example, the glucose transporter has a nucleic acid sequence of SEQ ID NO: 3. In another example, the glucose transporter encoded in the expression construct has a nucleic acid sequence that is 90%, 95%, 96%, 97%, 98%, 99%, or 99.9% identical to SEQ ID NO: 3. Irrespective of the sequence identity percentage, the glucose transporter is capable of transporting glucose.

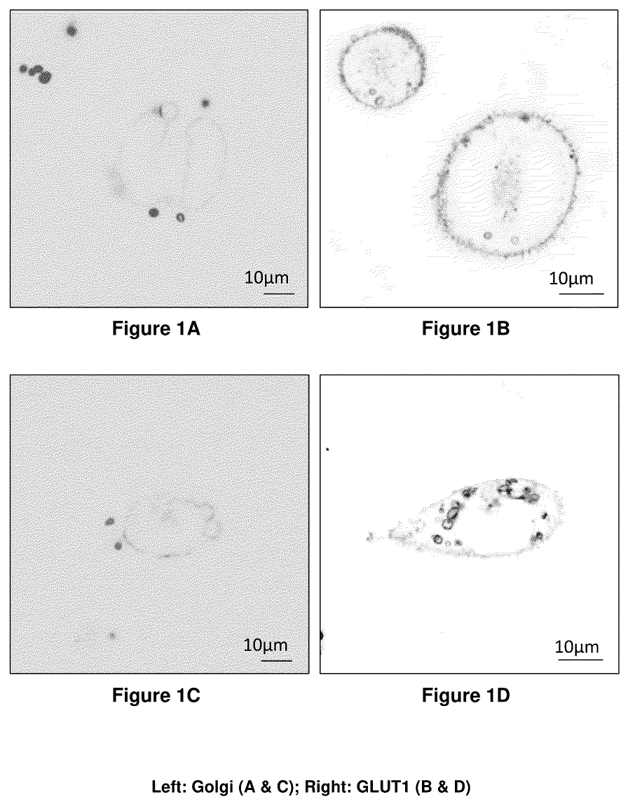

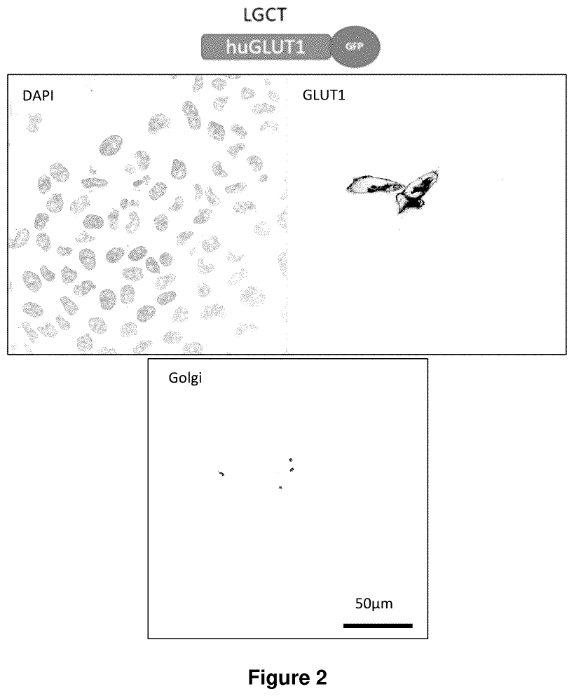

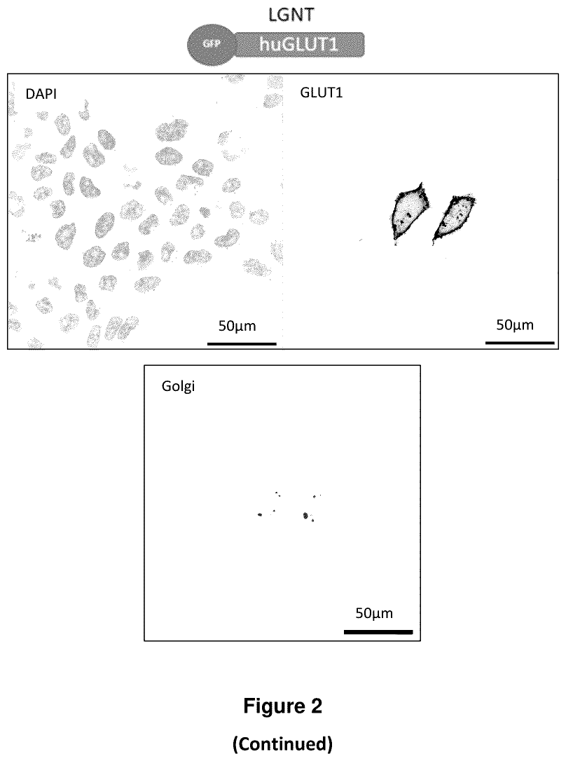

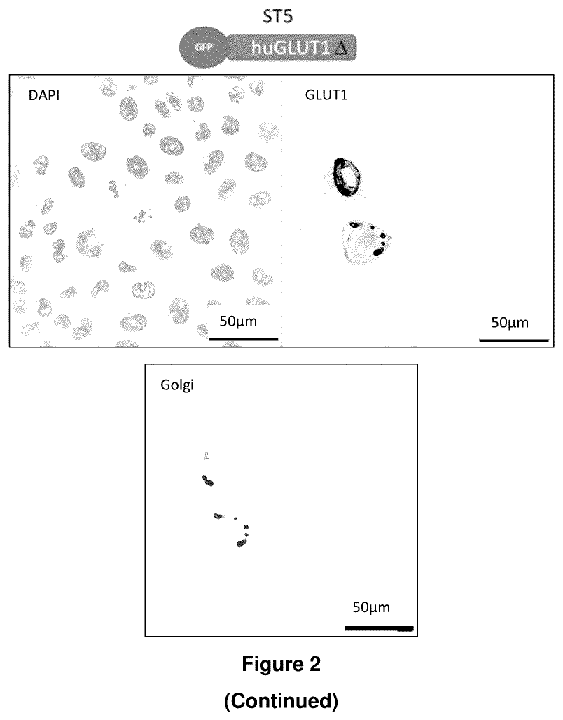

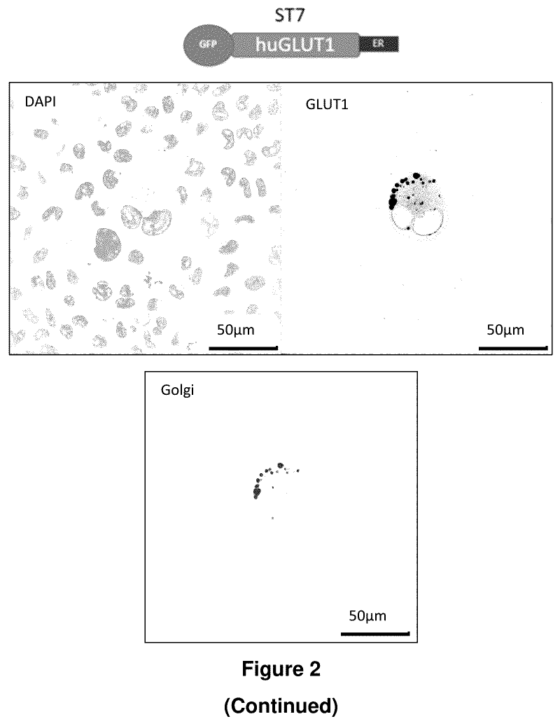

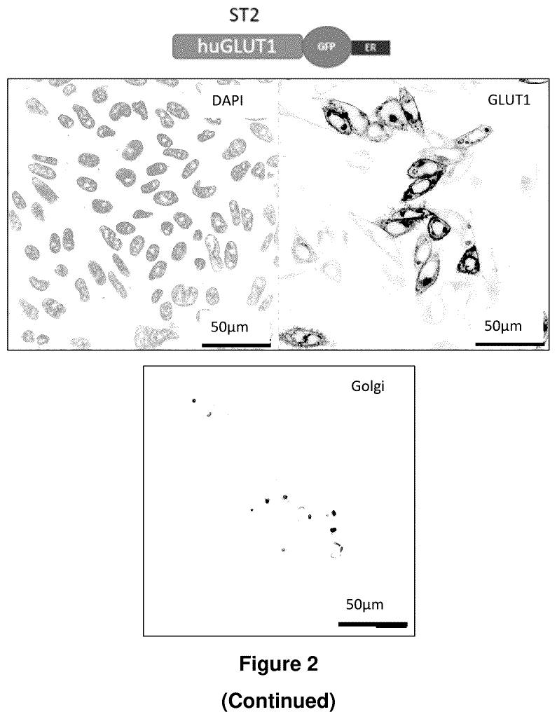

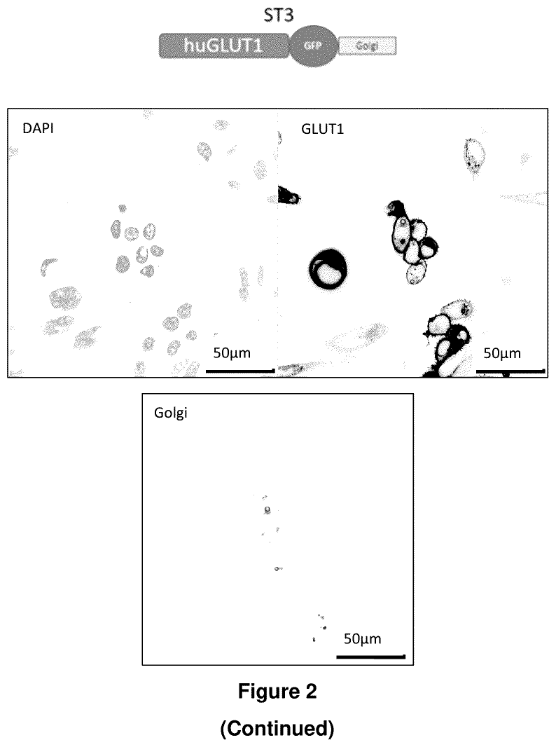

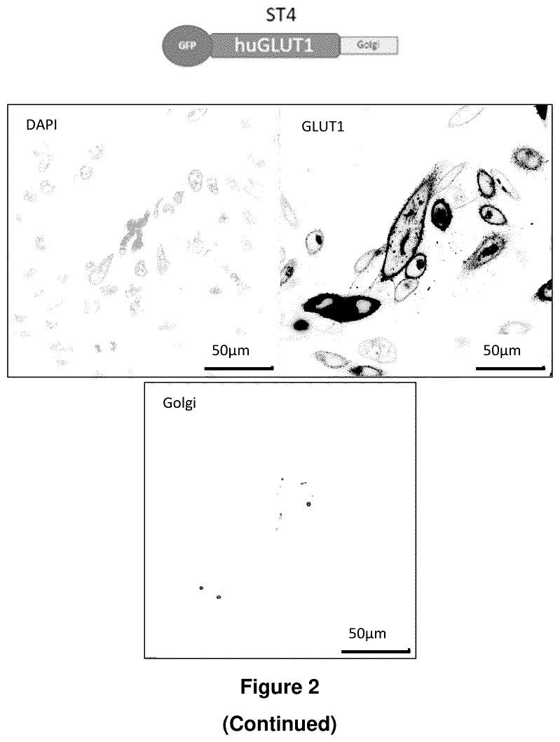

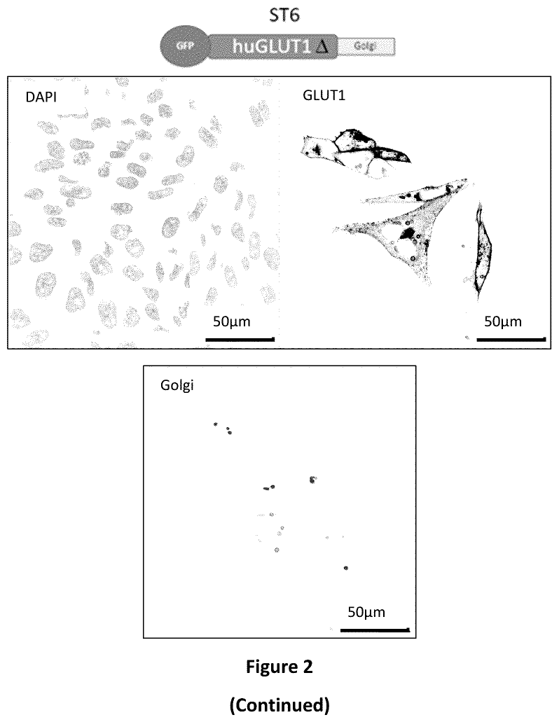

In one example, the glucose transporter comprised in the expression construct is a wild-type glucose transporter. In another example, the glucose transporter comprised in the expression construct is an engineered glucose transporter. As shown in FIG. 1B and FIG. 1D, the glucose transporter GLUT1 can be fused with a N-terminal or C-terminal Green Fluorescent Protein (GFP). The additional fusion protein may affect the subcellular localisation of the GLUT1 expressed in cells. Preferably, the GFP is tagged to GLUT1 on the C-terminal, which enriched the GLUT1 expressed in the Golgi apparatus or the ER. Other examples of engineering of GLUT1 are shown in FIG. 2. In one example, a full-length glucose transporter protein can be fused with a reporter protein at the N-terminal or the C-terminal. Additional signal sequences, such as Golgi retention sequence, or ER retention sequence, can be fused with the glucose transporter/reporter fusion protein. In one example, the signal sequences are fused at the C-terminal of the glucose transporter/reporter fusion protein. In one example, the ER retention sequence is LLTKVKGS (SEQ ID NO: 30). In another example, the Golgi retention sequence is PRQDTTSIQQGETASKERVIGV (SEQ ID NO: 31) or TTSIQQGETASKERVIGV (SEQ ID NO: 32).

In another example, a C-terminal truncated glucose transporter is used. In one example, the truncated glucose transporter has a nucleic acid sequence of SEQ ID NO: 4. In another example, the truncated glucose transporter encoded in the expression construct has a nucleic acid sequence that is 90%, 95%, 96%, 97%, 98%, 99%, or 99.9% identical to SEQ ID NO: 4. Irrespective of the sequence identity percentage, the truncated glucose transporter is capable of transporting glucose.

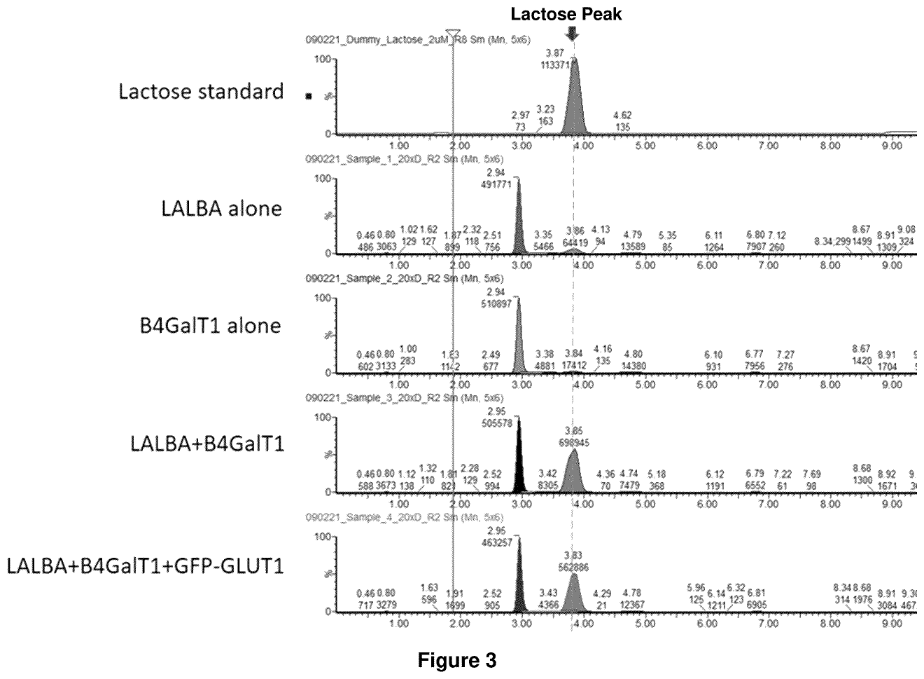

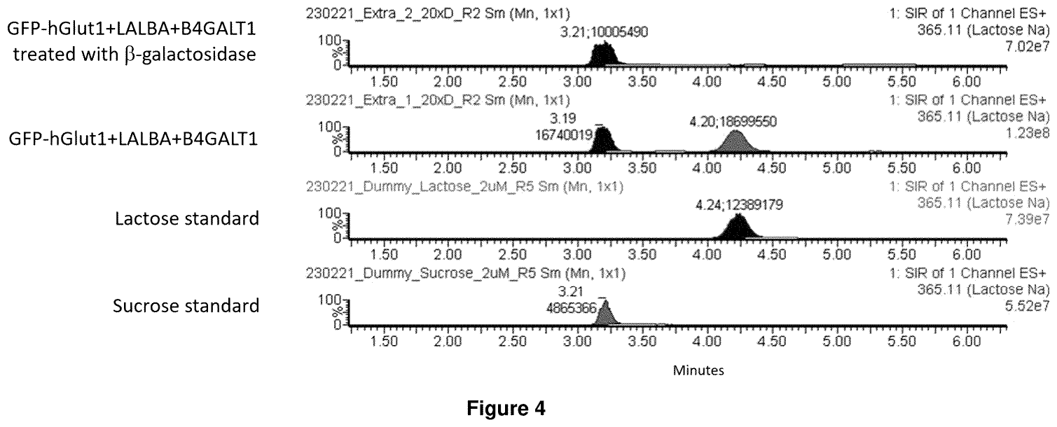

As shown in FIG. 3, recombinant cells comprise expression constructs comprising an alpha-lactalbumin (LALBA) and a beta-1,4-galactosyltransferase 1 (B4GalT1) which produce lactose. In another example, recombinant cells comprise expression constructs comprising alpha-lactalbumin (LALBA), a beta-1,4-galactosyltransferase 1 (B4GalT1) and an exemplary glucose transporter GLUT1 which also produces lactose. The production of lactose is confirmed as shown in FIG. 4, which digested the lactose produced using the recombinant cells as disclosed herein with beta-galactosidase.

Various combinations of the expression constructs in the recombinant cells are tested as shown in FIG. 4A. To select for the recombinant cells expressing the expression constructs, sequence encoding one or more selective markers are comprised in the expression construct(s). Methods and systems for selecting cells based on selective markers are known by a person skilled in the art. A person skilled in the art would appreciate that the host cells used in combination with a particular selectable marker may contain necessary mutation(s) to enable the selection of successful transfectants. In one example, the selection is based on glutamine synthetase (GS) system. The selective marker sequence can be a nucleic acid sequence encoding a glutamine synthetase (GS) while the recombinant cell comprises a glutamine synthetase (GS)-knockout mutation (CHO GS^{-/-}) that provides disadvantage in survival for cells without the expression construct expressing the glutamine synthetase (GS). In another example, the selectable marker encodes dihydrofolate reductase (DHFR). In yet another example, the selectable marker encodes an antibiotic compound resistant marker. In a further example, the antibiotic compound can be a hygromycin B (O-6-Amino-6-deoxy-L-glycero-D-galacto-heptopyranosylidene-(1-2-3)-O-β-D-talopyranosyl(1-5)-2-deoxy-N3-methyl-D-streptamine), an ampicillin ((2S,5R,6R)-6-([(2R)-2-amino-2-phenylacetyl]amino)-3,3-dimethyl-7-oxo-4-thia-1-azabicyclo[3.2.0]heptane-2-carboxylic acid), or a kanamycin (2-(aminomethyl)-6-[4,6-diamino-3-[4-amino-3,5-dihydroxy-6-(hydroxymethyl)tetrahydropyran-2-yl]oxy-2-hydroxy-cyclohexoxy]-tetrahydropyran-3,4,5-triol).

In one example, the recombinant cell comprises: an expression construct that encodes an alpha-lactalbumin (LALBA), a beta-1,4-galactosyltransferase 1 (B4GalT1), a glucose transporter 1 (GLUT1), and a marker protein, wherein C-terminus of LALBA is linked to N-terminus of B4GalT1, C-terminus of B4GalT1 is linked to N-terminus of GLUT1, and C-terminus of GLUT1 is fused to N-terminus of the marker protein, and an expression construct that encodes a beta-1,4-galactosyltransferase 1 (B4GalT1) and an alpha-lactalbumin (LALBA), wherein C-terminus of B4GalT1 is linked to N-terminus of LALBA.

In another example, the recombinant cell comprises: an expression construct that encodes an alpha-lactalbumin (LALBA), a beta-1,4-galactosyltransferase 1 (B4GalT1), a marker protein, a glucose transporter 1 (huGLUT1), and an ER localization signal, wherein C-terminus of LALBA is linked to N-terminus of B4GalT1, C-terminus of B4GalT1 is linked to N-terminus of the marker protein, C-terminus of the marker protein is linked to N-terminus of GLUT1, and C-terminus of GLUT1 is linked to N-terminus of the ER localization signal, and an expression construct that encodes a beta-1,4-galactosyltransferase 1 (B4GalT1) and an alpha-lactalbumin (LALBA), wherein C-terminus of B4GalT1 is linked to N-terminus of LALBA.

In another example, the recombinant cell comprises: an expression construct that encodes an alpha-lactalbumin (LALBA), a beta-1,4-galactosyltransferase 1 (B4GalT1), a marker protein, and a glucose transporter 1 (GLUT1), wherein C-terminus of LALBA is linked to N-terminus of B4GalT1, C-terminus of B4GalT1 is linked to N-terminus of the marker protein, and C-terminus of the marker protein is linked to N-terminus of GLUT1, and an expression construct that encodes a beta-1,4-galactosyltransferase 1 (B4GalT1) and an alpha-lactalbumin (LALBA), wherein C-terminus of B4GalT1 is linked to N-terminus of LALBA.

In another example, the recombinant cell comprises: a first expression construct that encodes an alpha-lactalbumin (LALBA) and a second expression construct that encodes a beta-1,4-galactosyltransferase 1 (B4GalT1).

In another example, the recombinant cell comprises: an expression construct that encodes an alpha-lactalbumin (LALBA), a beta-1,4-galactosyltransferase 1 (B4GalT1), a marker protein, and a C-terminal truncated glucose transporter 1 (GLUT14), wherein C-terminus of LALBA is linked to N-terminus of B4GalT1, C-terminus of B4GalT1 is linked to N-terminus of the marker protein, and C-terminus of the marker protein is linked to N-terminus of GLUT1A and an expression construct that encodes a beta-1,4-galactosyltransferase 1 (B4GalT1) and an alpha-lactalbumin (LALBA), wherein C-terminus of B4GalT1 is linked to N-terminus of LALBA.

In another example, the recombinant cell comprises: an expression construct that encodes an alpha-lactalbumin (LALBA), a beta-1,4-galactosyltransferase 1 (B4GalT1), a marker protein, a C-terminal truncated glucose transporter 1 (GLUT14), and a Golgi localization sequence, wherein the C-terminus of LALBA is linked to N-terminus of B4GalT1, C-terminus of B4GalT1 is linked to N-terminus of the marker protein, C-terminus of the marker protein is linked to N-terminus of GLUT14, and C-terminus of GLUT14 is linked to N-terminus of the Golgi localization sequence, and an expression construct that encodes a beta-1,4-galactosyltransferase 1 (B4GalT1) and an alpha-lactalbumin (LALBA), wherein C-terminus of B4GalT1 is linked to N-terminus of LALBA.

In another example, the recombinant cell comprises: an expression construct that encodes an alpha-lactalbumin (LALBA), a beta-1,4-galactosyltransferase 1 (B4GalT1), a marker protein, a glucose transporter 1 (huGLUT1), and a Golgi localization sequence, wherein C-terminus of LALBA is linked to N-terminus of B4GalT1, C-terminus of B4GalT1 is linked to N-terminus of the marker protein, C-terminus of the marker protein is linked to N-terminus of GLUT1, and C-terminus of GLUT1 is linked to N-terminus of C-terminal of the Golgi localization sequence, and an expression construct that encodes a beta-1,4-galactosyltransferase 1 (B4GalT1) linked to alpha-lactalbumin (LALBA), wherein C-terminus of B4GalT1 is linked to N-terminus of LALBA.

In another example, the recombinant cell comprises: an expression construct that encodes an alpha-lactalbumin (LALBA), a beta-1,4-galactosyltransferase 1 (B4GalT1), a marker protein, and a glucose transporter 1 (GLUT1), wherein C-terminus of LALBA is linked to N-terminus of B4GalT1, C-terminus of B4GalT1 is linked to N-terminus of the marker protein, and C-terminus of the marker protein is linked to N-terminus of GLUT1.

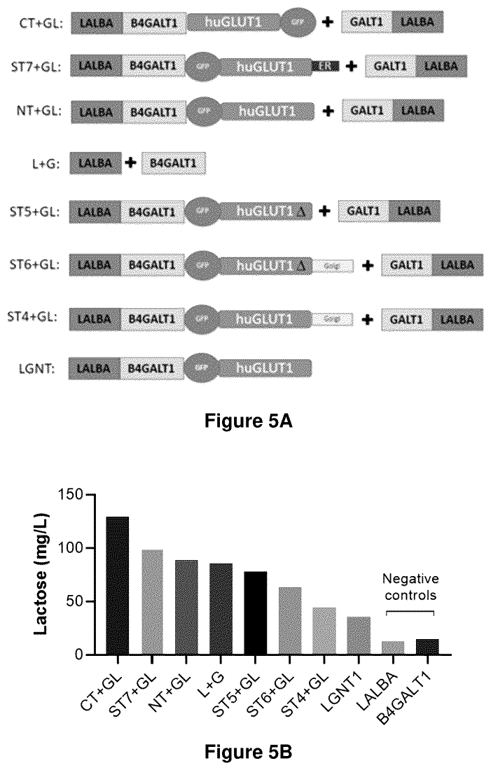

The lactose production levels in these exemplary embodiments are shown in FIG. 5B.

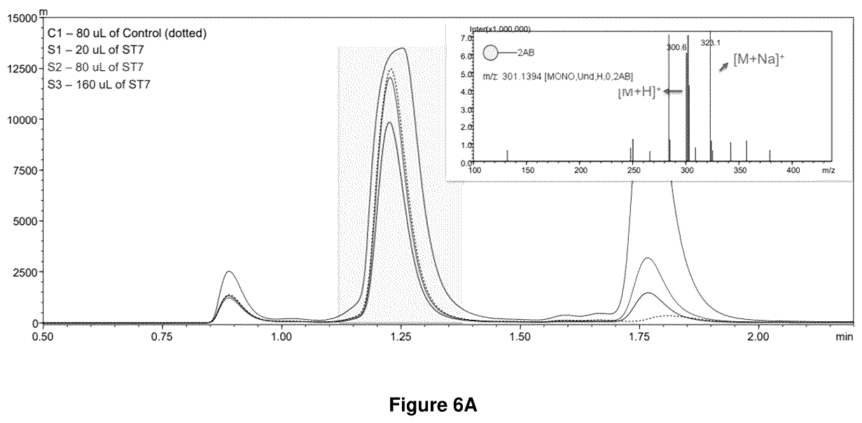

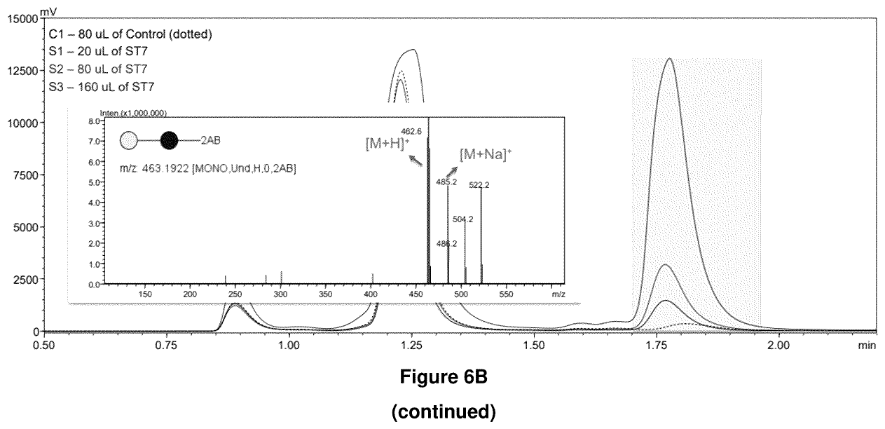

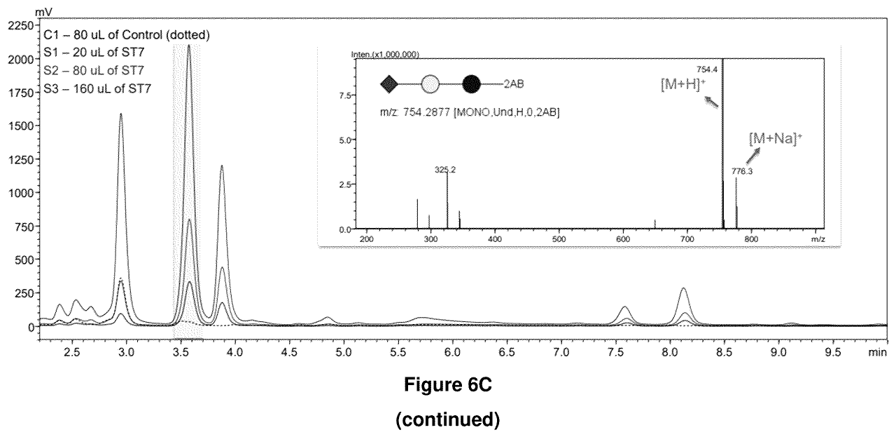

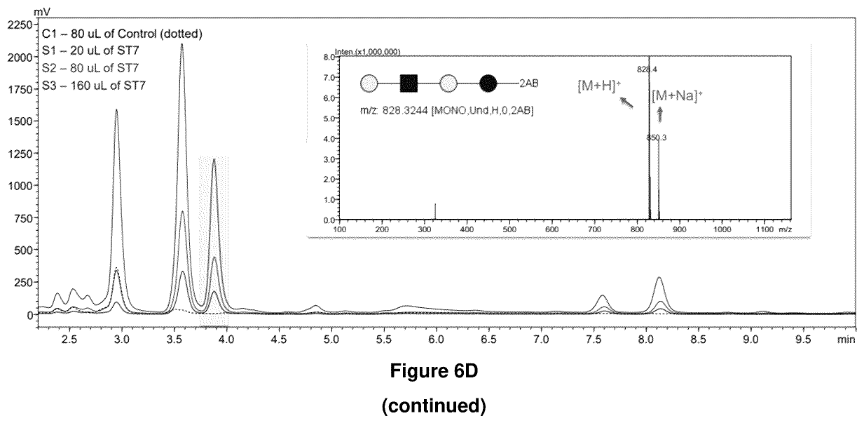

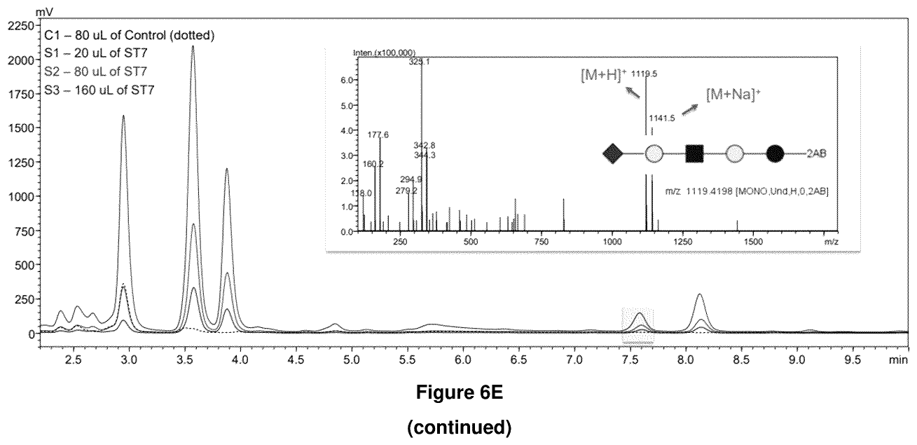

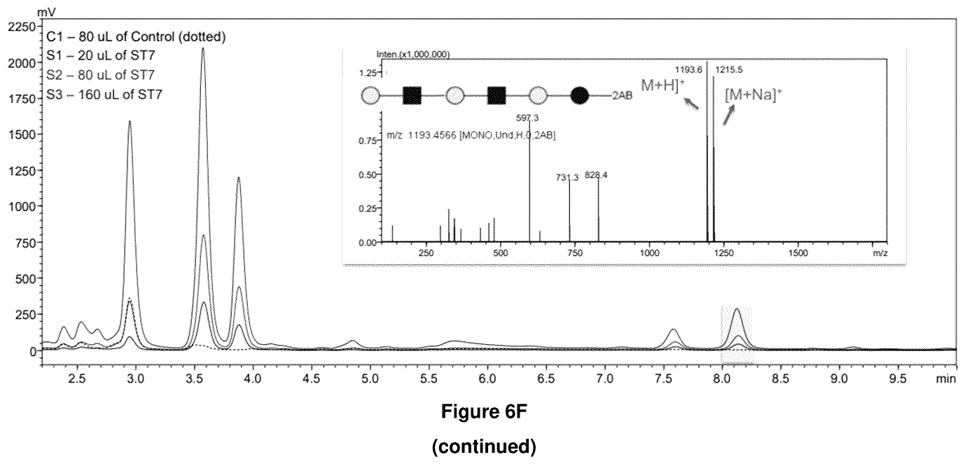

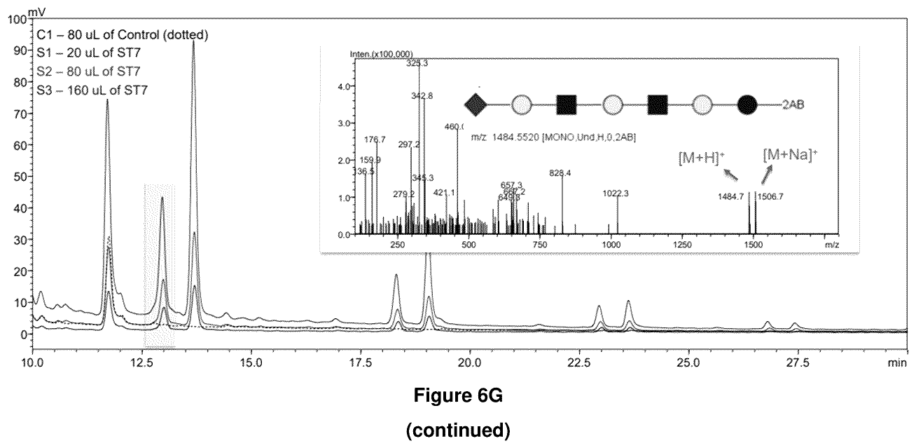

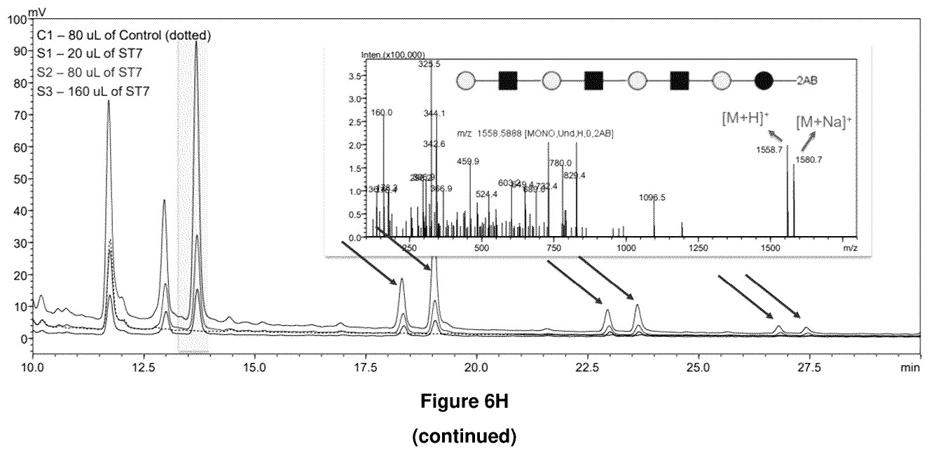

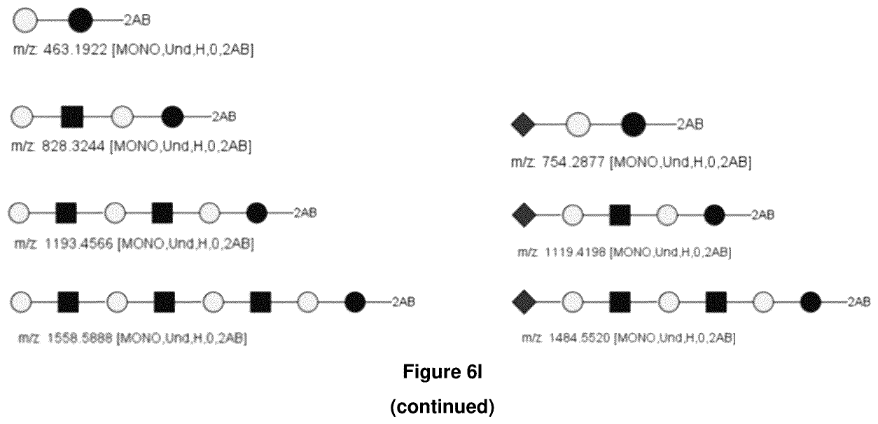

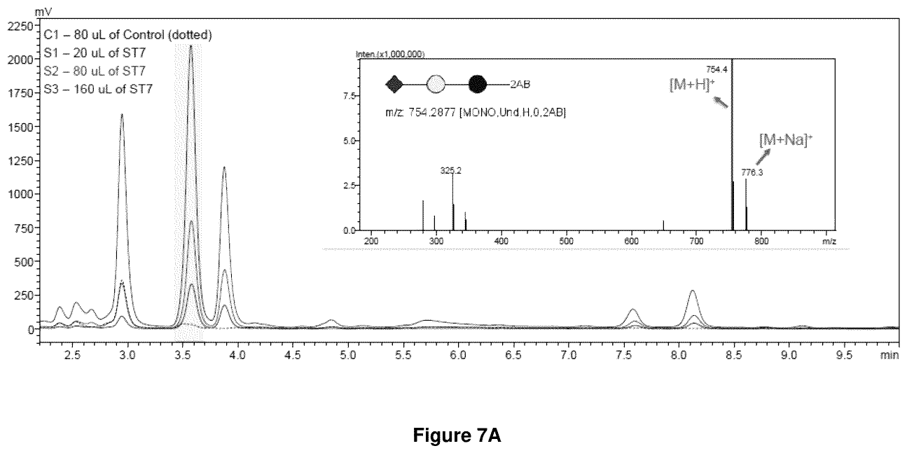

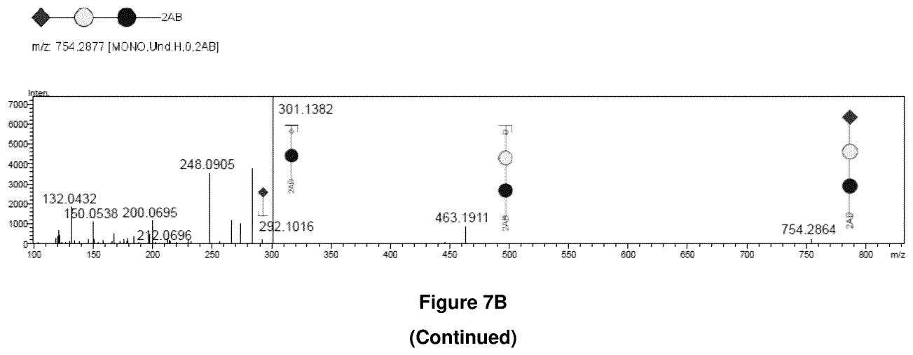

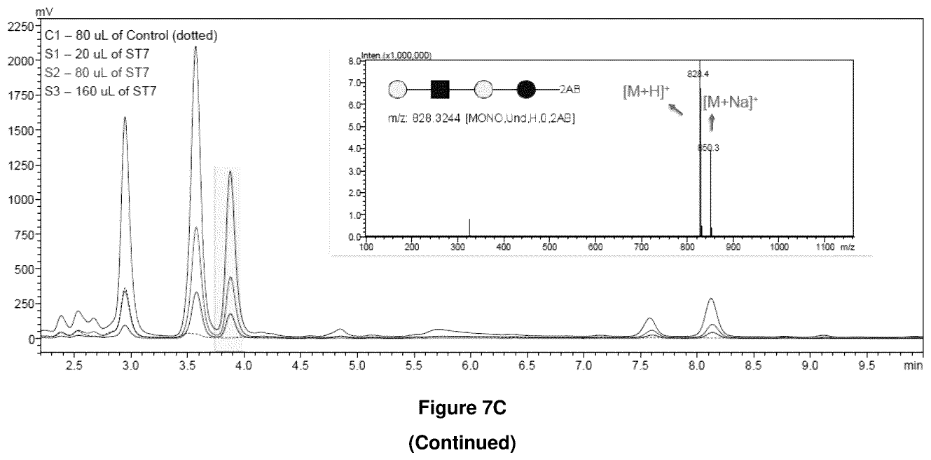

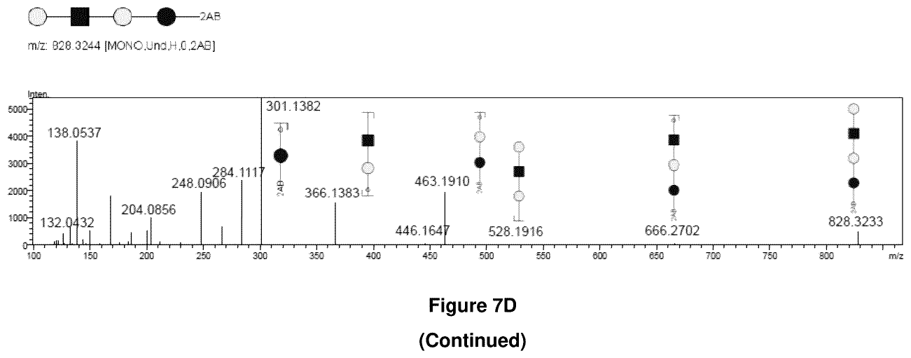

Lactose produced by recombinant cells are further processed into milk oligosaccharides within the same recombinant cells. As described in FIG. 6C to FIG. 6I, several milk oligosaccharides commonly found in human breast milk are identified in the cell culture media of the recombinant cells. Thus, in one embodiment, the recombinant cell for producing lactose further produces within said cell human milk oligosaccharides (HMOs). Human milk oligosaccharides comprises, and are not limited to: 2′-Fucosyllactose (2′-FL), 3′-Fucosyllactose (3′-FL), Lacto-N-tetraose (LNT), Lacto-N-neotetraose (LNnT), Lacto-N-fucopentaose I (LNFPI), Lacto-N-fucopentaose II (LNFPII), Lacto-N-fucopentaose III (LNFPIII), 3′-Sialyllactose (3′-SL), 6′-Sialyllactose (6′-SL), Sialyllacto-N-tetraose (a) (LSTa), Sialyllacto-N-tetraose (b) (LSTb), Sialyllacto-N-tetraose (c) (LSTc), 6′-Sialyllactosamine (6′ SLN), Disialyllactose (DSL), Disialyllactose-N-tetraose (DSLNT), α-3′-Galactosyllactose (a3′-GL), β-3′-Galactosyllactose (33′-GL), 3-4′-Galactosyllactose (4′-GL), β-6′-Galactosyllactose (6′-GL), α-3′-N-acetylgalactosaminyllactose (α-3′-GalNACL), Lacto-N-difucohexaose I (LNDFH-I), Lacto-N-neohexaose (LNnH), Lacto-N-hexaose (LNH), and 6′-N-Acetyl-glucosaminyl-lactose (NAL). For example, the detected human milk oligosaccharides (HMOs) comprise Sialyl-lactose, Lacto-n-neotatraose (LNnT), sialyl-LNnT, para-lacto-N-hexaose (Para-LNH), sialyl-para-LNH, para-lacto-N-octaose. In a further example, the sialyl-lactose is a 3′ sialyl-lactose (3′SL).

In another aspect, the disclosure provides a one or more expression constructs. In one example, the one or more expression constructs are the same constructs comprised in the recombinant cells as described herein. In one example, the one or more expression constructs are plasmids. In a further example, the one or more expression constructs are in the form of circular DNA. A person skilled in the art would understand that the one or more expression constructs serve the purpose of expressing recombinant proteins in a system. Thus, the expression constructs comprise, but are not limited to, a vector backbone, one or more origins of replication, a selection marker, a reporter (for example, a fluorescent protein), a promoter, an internal ribosome entry site (IRES), a linker sequence, and multiple cloning sites. The one or more expression constructs can utilise the same or different vector backbones, such as pcDNA3.1, pGenLenti, and pCMV that are commonly known in the art.

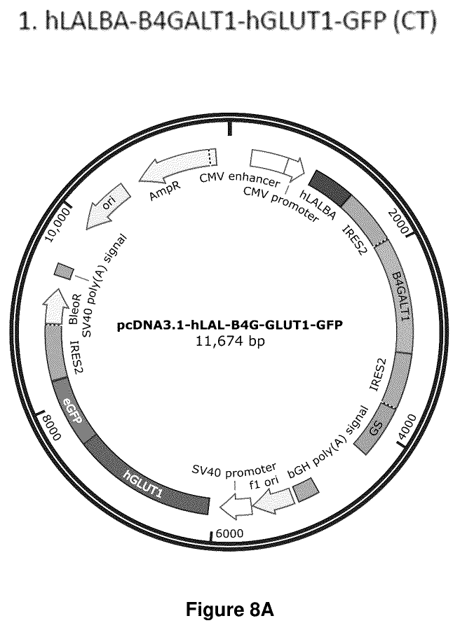

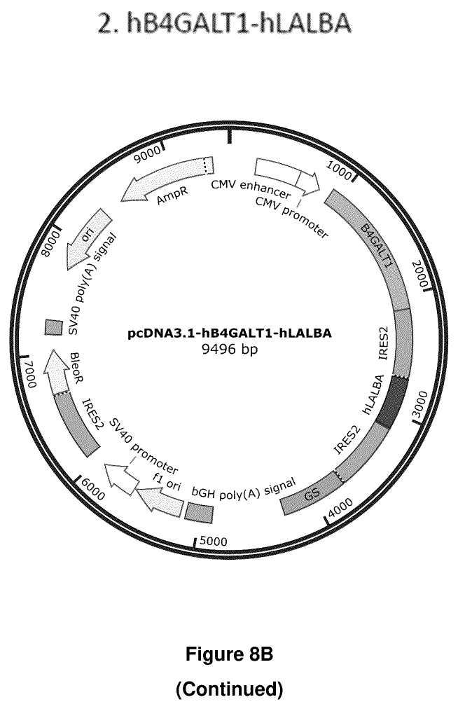

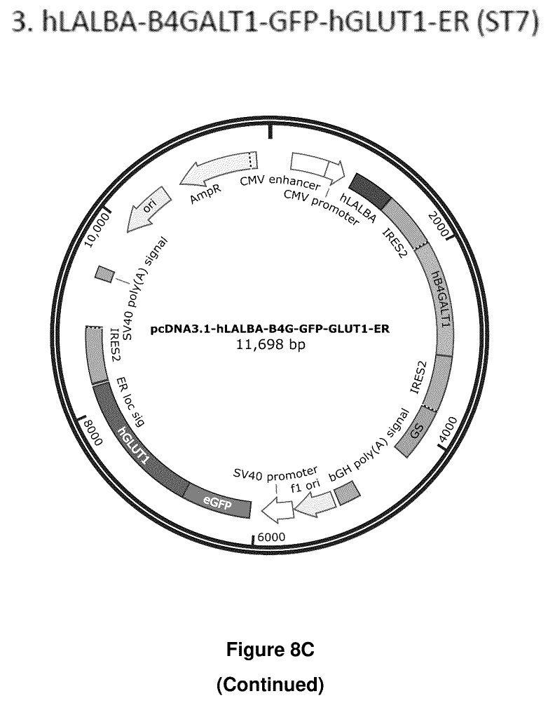

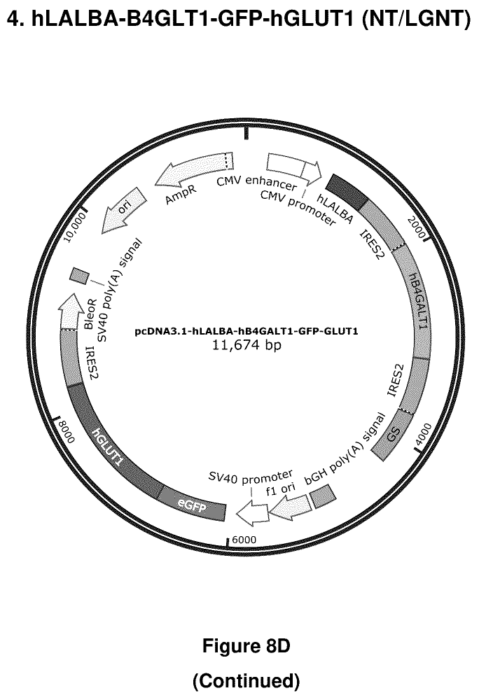

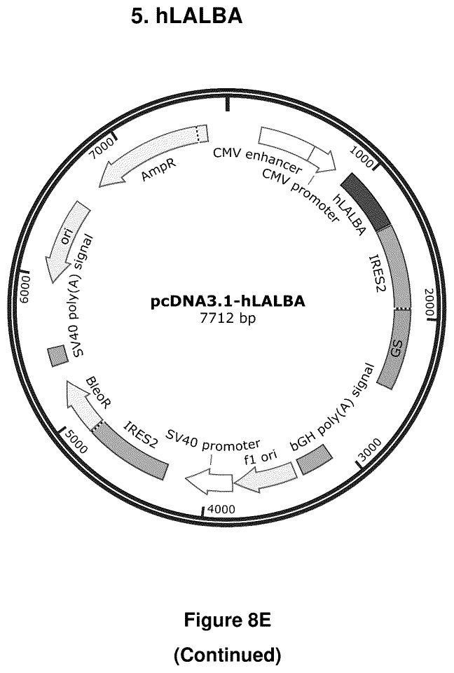

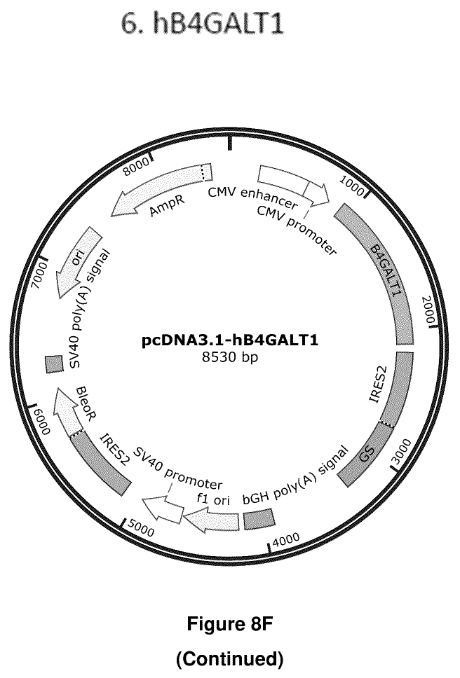

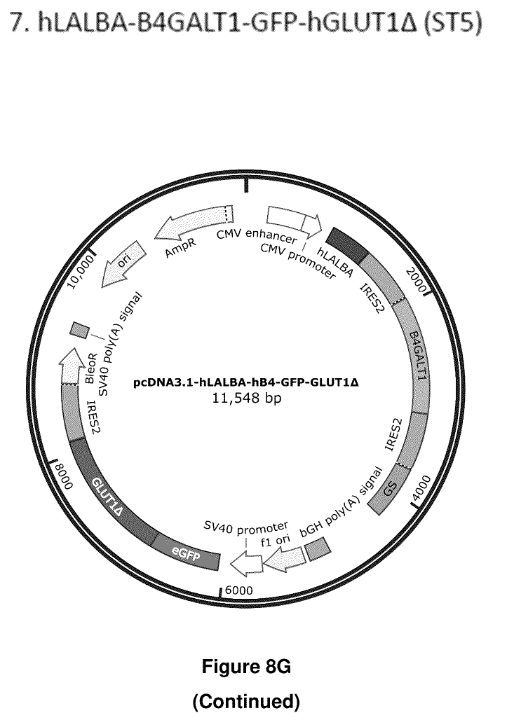

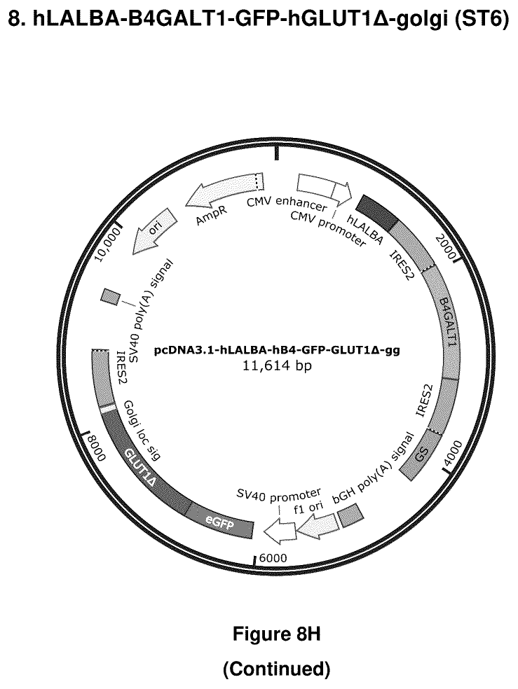

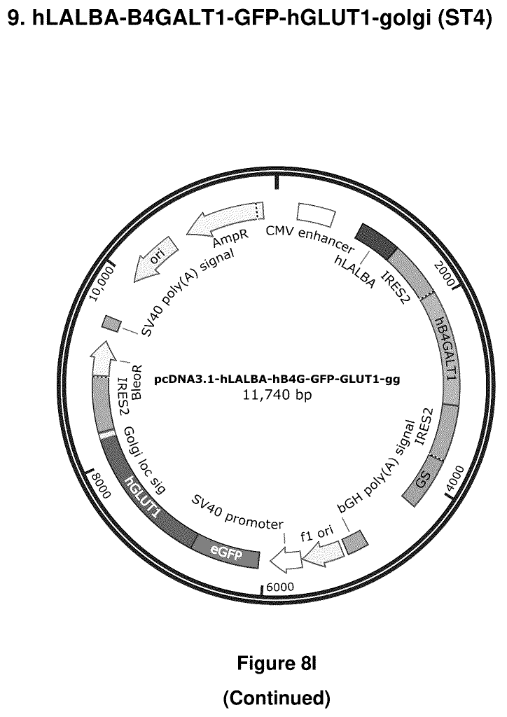

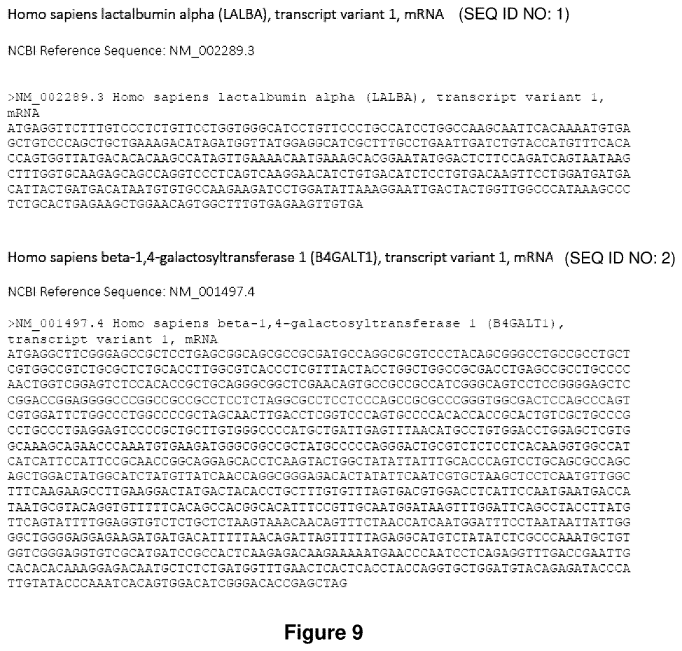

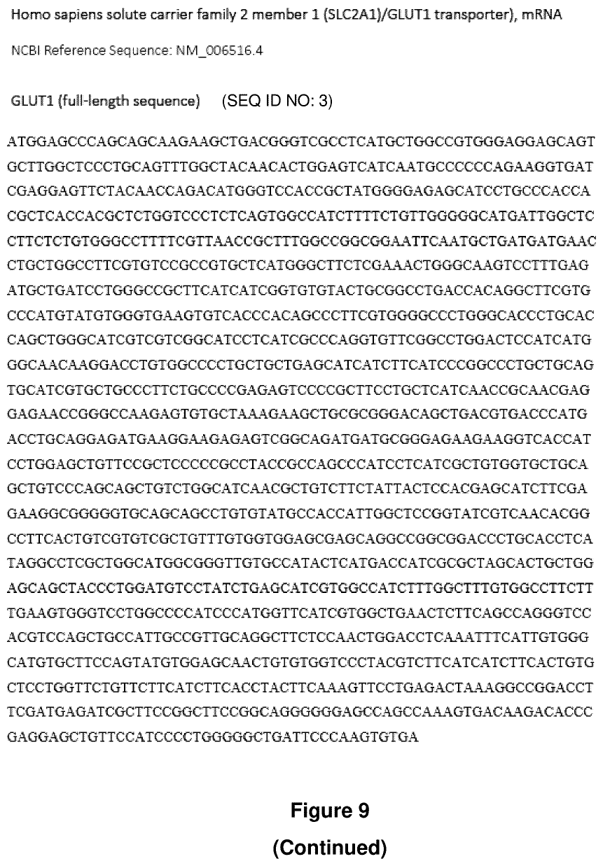

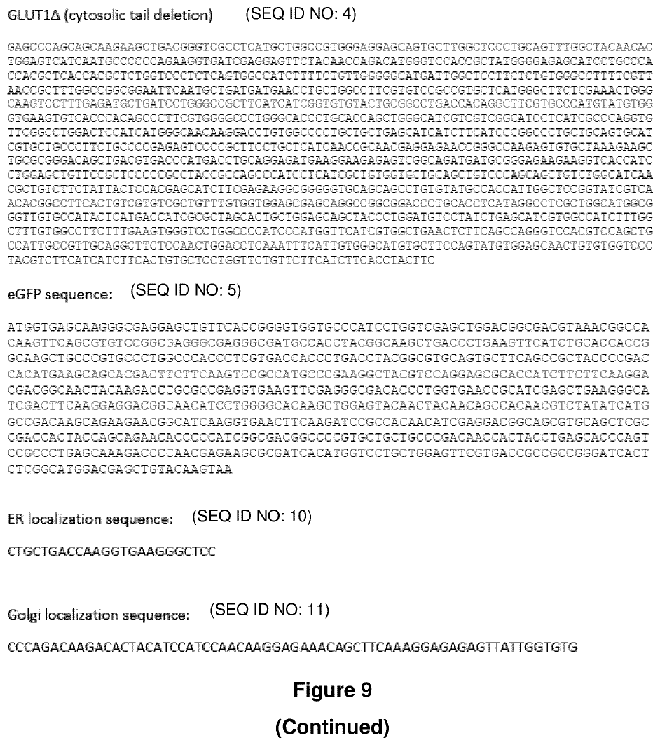

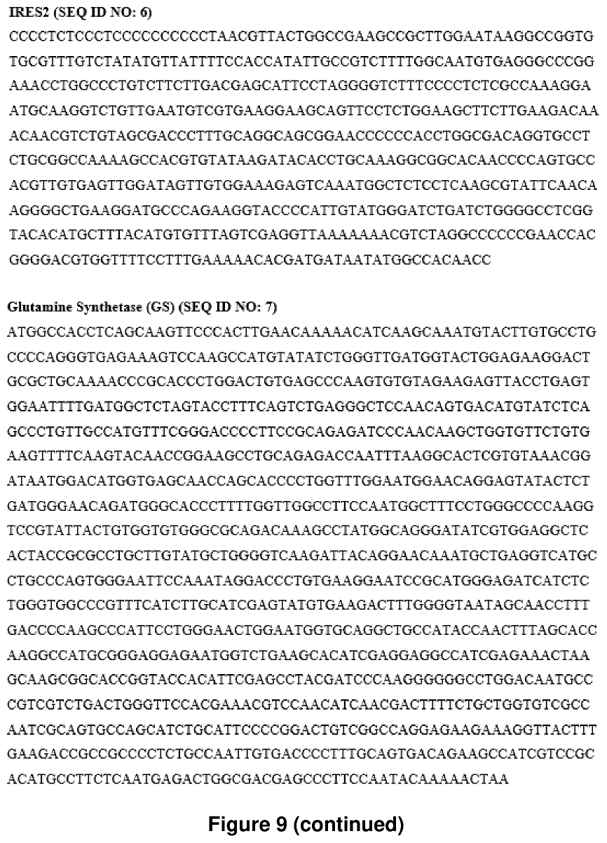

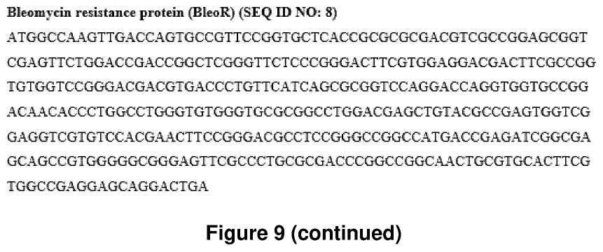

In one embodiment, the one or more expression constructs comprise polynucleotides encoding an alpha-lactalbumin (LALBA) and a beta-1,4-galactosyltransferase 1 (B4GalT1). In one example, the one or more expression constructs further comprise polynucleotide sequences encoding a glucose transporter. In one example, the one or more expression constructs comprise polynucleotide sequences encoding an alpha-lactalbumin (LALBA), and a beta-1,4-galactosyltransferase 1 (B4GalT1), and optionally a glucose transporter. In one further example, the one or more expression constructs comprise polynucleotide sequences encoding an alpha-lactalbumin (LALBA) and a beta-1,4-galactosyltransferase 1 (B4GalT1) and optionally a glucose transporter are engineered in at least one, at least two, at least three or at least four expression constructs. Exemplary expression constructs are shown in FIG. 8A to FIG. 8I.

In one example, the polynucleotides encoding an alpha-lactalbumin (LALBA) and the polynucleotide encoding a beta-1,4-galactosyltransferase 1 (B4GalT1) are within the same construct. In another example, the polynucleotides encoding an alpha-lactalbumin (LALBA) and polynucleotides encoding a beta-1,4-galactosyltransferase 1 (B4GalT1) are in separate expression constructs. In another example, the polynucleotides encoding alpha-lactalbumin (LALBA) and beta-1,4-galactosyltransferase 1 (B4GalT1) encoded are of mammalian origin. In a further example, the polynucleotides encoding alpha-lactalbumin (LALBA) and beta-1,4-galactosyltransferase 1 (B4GalT1) are of human origin. In a further example, the polynucleotides encoding alpha-lactalbumin (LALBA) and beta-1,4-galactosyltransferase 1 (B4GalT1) are from a hamster. In yet another example, the polynucleotides encoding alpha-lactalbumin (LALBA) and beta-1,4-galactosyltransferase 1 (B4GalT1) are from the same species. In yet another example, polynucleotides encoding the alpha-lactalbumin (LALBA) and beta-1,4-galactosyltransferase 1 (B4GalT1) are from the same genus. In yet another example, the polynucleotides encoding alpha-lactalbumin (LALBA) and beta-1,4-galactosyltransferase 1 (B4GalT1) are from different species. The alpha-lactalbumin (LALBA) and beta-1,4-galactosyltransferase 1 (B4GalT1) encoded in the expression construct may be expressed under the control of the same promoter, or different promoters. The alpha-lactalbumin (LALBA) and beta-1,4-galactosyltransferase 1 (B4GalT1) encoded in the expression construct may be tagged or untagged, by a reporter. The reporter sequence tagging the alpha-lactalbumin (LALBA) and/or beta-1,4-galactosyltransferase 1 (B4GalT1) may be at the N-terminal, or at the C-terminal of each polynucleotide. In one example, the reporter is a fluorescent protein. In a further example, the fluorescent protein is a Green Fluorescent Protein (GFP).

In another example, the polynucleotide encoding alpha-lactalbumin (LALBA) has a nucleic acid sequence of SEQ ID NO: 1. In another example, the polynucleotide encoding beta-1,4-galactosyltransferase 1 (B4GalT1) has a nucleic acid sequence of SEQ ID NO: 2. In another example, the polynucleotide encoding alpha-lactalbumin (LALBA) has a nucleic acid sequence that is 90%, 95%, 96%, 97%, 98%, 99%, or 99.9% identical to SEQ ID NO: 1. In another example, the polynucleotide encoding beta-1,4-galactosyltransferase 1 (B4GalT1) has a nucleic acid sequence that is 90%, 95%, 96%, 97%, 98%, 99%, or 99.9% identical to SEQ ID NO: 2. Irrespective of the sequence identity percentage, the alpha-lactalbumin (LALBA) and the beta-1,4-galactosyltransferase 1 (B4GalT1) are capable of lactose synthesis.

In another embodiment, the one or more expression constructs further comprise a polynucleotide encoding a glucose transporter. In one example, the polynucleotide encoding the glucose transporter can be in a different expression construct with the one or more expression constructs comprising polynucleotides encoding alpha-lactalbumin (LALBA) and/or beta-1,4-galactosyltransferase 1 (B4GalT1). In another example, the polynucleotide encoding the glucose transporter can be in the same expression construct with the one or more expression constructs comprising polynucleotide encoding alpha-lactalbumin (LALBA) or beta-1,4-galactosyltransferase 1 (B4GalT1), or both. In one example, the glucose transporter is a mammalian glucose transporter. In another example, the glucose transporter is a human glucose transporter. In a further example, the glucose transporter is from GLUT family. In another example, the glucose transporter is selected from any one of the following: GLUT 1, GLUT2, GLUT3, GLUT4, GLUT5, GLUT6, GLUT7, GLUT8, GLUT9, GLUT10, GLUT11, GLUT12, HMIT (H+ driven myoinositol transporter, also GLUT13), GLUT14, and sodium-glucose transporter (SGLT1). In yet another example, the glucose transporter is GLUT1. In one example, the glucose transporter has a nucleic acid sequence of SEQ ID NO: 3. In another example, the glucose transporter encoded in the expression construct has a nucleic acid sequence that is 90%, 95%, 96%, 97%, 98%, 99%, or 99.9% identical to SEQ ID NO: 3. Irrespective of the sequence identity percentage, the glucose transporter is capable of transporting glucose.

In one example, the glucose transporter comprised in the expression construct is a wild-type glucose transporter. In another example, the glucose transporter comprised in the expression construct is an engineered glucose transporter. In one example, a full-length glucose transporter sequence can be fused with a reporter sequence at the N-terminal or the C-terminal. Additional signal sequences, such as Golgi retention sequence, or ER retention sequence, can be fused with the glucose transporter/reporter fusion polynucleotide sequence. The signal sequences are preferably fused at the C-terminal of the glucose transporter/reporter polynucleotide sequence. In one example, the ER retention sequence is LLTKVKGS (SEQ ID NO: 30). In another example, the Golgi retention sequence is PRQDTTSIQQGETASKERVIGV (SEQ ID NO: 31) or TTSIQQGETASKERVIGV (SEQ ID NO: 32).

In another embodiment, a C-terminal truncated glucose transporter is used. In one example, the truncated glucose transporter has a nucleic acid sequence of SEQ ID NO: 4. In another example, the truncated glucose transporter encoded in the expression construct has a nucleic acid sequence that is 90%, 95%, 96%, 97%, 98%, 99%, or 99.9% identical to SEQ ID NO: 4.

In another embodiment, the one or more expression constructs further comprise sequences encoding one or more selectable markers in the expression constructs to allow selection of the host cell with the one or more expression construct. Methods and systems for selecting cells based on selectable markers are known by a person skilled in the art. In one example, the selection is based on glutamine synthetase (GS) system. The selectable marker sequence can be a nucleic acid sequence encoding a glutamine synthetase (GS), a dihydrofolate reductase (DHFR) or an antibiotic compound resistant marker. A person skilled in the art would appreciate that the host cells used for a particular selectable marker may contain necessary mutations to enable the selection. For example, to select cells successfully transfected with expression constructs comprising glutamine synthetase (GS), the host cells may comprise a GS^{-/-} knockout mutation.

In another embodiment, the one or more expression constructs comprise a sequence having at least 90% or 95% or 98% or 100% sequence identity with SEQ ID NO: 1 (alpha-lactalbumin (LALBA)). In another embodiment, the one or more expression constructs comprise a sequence having at least 90% or 95% or 98% or 100% sequence identity with SEQ ID NO: 2 (beta-1,4-galactosyltransferase 1 (B4GalT1)). In another embodiment, the one or more expression constructs comprise a sequence having at least 90% or 95% or 98% or 100% sequence identity with SEQ ID NO: 3 (glucose transporter 1 (GLUT1)). In another embodiment, the one or more expression constructs comprise a sequence having at least 90% or 95% or 98% or 100% sequence identity with SEQ ID NO: 4 (C-terminal truncated glucose transporter 1 (GLUT14)). In yet another embodiment, the one or more expression constructs comprise the following: a sequence having at least 90% or 95% or 98% or 100% sequence identity with SEQ ID NO: 1 (alpha-lactalbumin (LALBA)); a sequence having at least 90% or 95% or 98% or 100% sequence identity with SEQ ID NO: 2 (beta-1,4-galactosyltransferase 1 (B4GalT1)); and a sequence having at least 90% or 95% or 98% or 100% sequence identity with SEQ ID NO: 3 (glucose transporter 1 (GLUT1)). In yet another embodiment, the one or more expression constructs comprise the following: a sequence having at least 90% or 95% or 98% or 100% sequence identity to SEQ ID NO: 1 (alpha-lactalbumin (LALBA)); a sequence having at least 90% or 95% or 98% or 100% sequence identity to SEQ ID NO: 2 (beta-1,4-galactosyltransferase 1 (B4GalT1)); and a sequence having at least 90% or 95% or 98% or 100% sequence identity to SEQ ID NO: 4 (C-terminal truncated glucose transporter 1 (GLUT1Δ)).

In another aspect, the disclosure provides a method of producing lactose using a recombinant cell as described herein. In one embodiment, the disclosure provides a method of producing lactose using the recombinant cell, wherein the method comprises culturing the recombinant cell as described herein. It is appreciated by a person skilled in the art that the type of cell culture used depends on the type of the recombinant cell. Suitable cell culture media are well known in the art. In one example, the cell culture used for the CHO recombinant cell is a suspension cell culture. In the cell culture used for the CHO recombinant cell is an adherent cell culture. In another embodiment, the disclosure provides a method of producing lactose using the recombinant cell, wherein the method comprises culturing the recombinant cell as described herein, and detecting lactose from the recombinant cell culture media. As shown in FIG. 3 and FIG. 4, for example, a lactose peak is detected in the suspension culture.

In another aspect, the disclosure provides a cell culture comprising the recombinant cell as described herein, and a cell culture medium.

In another aspect, the disclosure provides a cryopreserved cell culture comprising the recombinant cell as described herein. Methods of cryopreserving cells are known in the art.

In another aspect, the disclosure provides a kit comprising the recombinant cell as described herein, or the cryopreserved cell culture as described herein.

In yet another aspect, the disclosure provides a kit comprising the one or more constructs as described herein. In one embodiment, the kit further comprises suitable cells for expression of the one or more constructs as described herein.

The disclosure has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein. Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

### EXAMPLES

Generating huGLUT1 Constructs for Transportation of Glucose from Cytosol to the ER or Golgi Apparatus in Cultured Cells

As glucose transporter 1 (GLUT1) is normally expressed on the cell surface and responsible for transporting glucose from the environment into the cells, GLUT1 variants were generated to identify a suitable variant that remains in the ER or Golgi, instead of translocating to the cell membrane. Such variant will be able to transport glucose from the cytosol to the ER or Golgi. To visualize the change in cellular localization of the GLUT1 in CHO cells, two GFP fusion proteins were produced with GLUT1, GFP-GLUT1 (GFP is fused to the N-terminus of GLUT1) and GLUT-GFP (GFP is fused to the C-terminus of GLUT1), respectively. The GLUT1/GFP fusion proteins were expressed in mammalian cell culture, for example, CHO cells. After transfecting the constructs encoding these two fusion proteins into suspension CHO cells, the GFP fused at the N-terminus of GLUT1 (GFP-GLUT1) did not seem to affect the cell membrane localization of GLUT1. However, the GFP fused at the C-terminus of GLUT1 affected the GLUT1 localization significantly (FIG. 1). This observation agrees with the notion that the cytosolic tail at the C-terminus plays a role in directing the localization of GLUT1. Thus, without being bound by theory, the results suggest that GLUT1-GFP is able to transport glucose to the ER or Golgi.

Additional huGLUT1 variants were generated and their cellular localization were analyzed in adherent CHO cells. Based on the observations shown in FIG. 1, several GLUT1 variants were generated and expressed in adherent CHO cells for detailed localization analyses. The C-terminal cytosolic tail of huGLUT1 was believed to play a role in directing the GLUT1 to the cell membrane. Therefore, the C-terminal cytosolic tail of huGLUT1 was deleted in one of the variants, GFP-GLUT1Δ. The ER retention signal of huGT2, LLTKVKGS (SEQ ID NO: 30), was fused to the C-terminus of the full length GLUT1, GFP-GLUT1-ER. The C-terminal 18 amino acids of the human CMP-sialic acid transporter (CST) (having the sequence of TTSIQQGETASKERVIGV (SEQ ID NO: 32)) were shown to play a critical role in directing the CST to the Golgi; these 18 amino acids were fused to the C-terminal ends of GFP-GLUT1 and GFP-GLUT1Δ. They have been named as GFP-GLUT1-Golgi and GFP-GLUT1Δ-Golgi respectively. The exemplary constructs were transiently expressed in adherent CHO cells and the cellular localization of the GFP fusion proteins (green) and the Golgi marker (red) were visualized by confocal microscopy. The results are shown in FIG. 2.

The data show that ST7 (GFP-huGLUT1-ER), ST5 (GFP-huGLUT1Δ) and some of ST6 (GFP-huGLUT1Δ-Golgi) are co-localized with the Golgi marker, suggesting Golgi localization. These constructs perform better in transporting glucose into the ER and Golgi.

### Production of Lactose in Cultured Cells Expressing Both α-Lactalbumin (LALBA) and B4GalT1

The lactose synthase (LS) enzyme synthesizes lactose (galactose β-1,4-glucose) from UDP-galactose and glucose. Lactose synthesis takes place in the Golgi of the mammary gland epithelial cells. The LS is an enzymatic complex of galactosyltransferase (B4GalT1) and alpha-lactalbumin (LALBA). LALBA is only found in mammary epithelial cells and it can increase the affinity of B4GalT1 for glucose by 1000-fold. Therefore, it enables B4GalT1 to add galactose from UDP-galactose to glucose even at low concentrations of glucose.

Expression constructs were generated to express human B4GalT1, human LALBA, and GFP-GLUT1. Different combinations of the three constructs were stably transfected into CHO cells. The presence of lactose in conditioned media was analysed by LC-MS and the results are shown in FIG. 3. The result showed that a molecule with the same mass as a disaccharide was eluted at the same position as lactose. The synthesis of this disaccharide requires both B4GalT1 and LALBA. CHO cells transfected with only one of them, either B4GalT1 alone or LALBA alone, were unable to produce this disaccharide, suggesting this disaccharide is lactose.

### Confirmation of Lactose Production in Recombination Cells by β-Galactosidase Treatment

The disaccharide shown in FIG. 3 is lactose as it was eluted out of the column at the same position as the lactose standard and has the same molecular mass as lactose. In addition, its synthesis requires both B4GalT1 and LALBA. To further confirm this, the product was treated with β-galactosidase which digests lactose. Indeed, the product was sensitive to β-galactosidase treatment thus resulting in loss of the lactose peak (FIG. 4). These results together confirmed that lactose was indeed produced in CHO with the expression of B4GalT1 and LALBA.

### Different GLUT1 Variants Resulted in Varied Amounts of Lactose Production in CHO Cells

With the confirmation of lactose production in CHO cells, further attempts were made to increase the productivity of lactose by optimizing the huGLUT1 constructs. Several huGLUT1 constructs and B4GalT1-LALBA constructs were generated and transfected into CHO cells. Stably transfected pools were developed, and the amounts of lactose produced by each pool was determined. The results are shown in FIG. 5.

Production of Putative HMOs in CHO-K1 Cells Transfected with huGLUT1, B4GalT1 and LALBA

The CHO-K1 ST7+GL (ST7) cells shown in FIG. 5 were cultivated in single-use Erlenmeyer flasks (Corning) with a cell seeding density of 0.3×106 cells/mL in 50/50 media. Cell culture supernatant was harvested on day 4 by centrifugation at 200 g for 5 minutes. Clarified supernatant was then applied to Blotglyco beads and columns (BlotGlyco Kit, Shimadzu) for glycan labelling and analysis, according to manufacturer's protocol. Briefly, 20-160 μL of clarified supernatant was allowed to bind to BlotGlyco beads by incubating for 1 hour at 80° C. until beads were dry. Beads were then washed twice with 2M guanidine hydrochloride, once with distilled water and twice with 1% trimethylamine/methanol solution by centrifugation through a column at 3000 g for 30 seconds. After blocking and wash steps, glycans were released using 2% acetic acid/acetonitrile, and labelled with fluorescent label 2-aminobenzamide (2-AB). 2-AB labelled glycans were washed to remove excess labelling reagent, collected and analysed with a liquid chromatograph coupled with fluorescence detector and quadruple time-of-flight mass spectrometer (LC-FLD-QTOF). The HMOs attached with 2-AB fluorescence label at the reducing ends enabled detection via fluorescence detector. Peaks present in ST7 cells, not in the untransfected control cells, from the LC-FLD chromatogram were then identified based on their respective MS spectra collected with the QTOF mass spectrometer under positive electrospray ionization mode.

The results showed that the ST7 cells produced 6 putative HMOs, with 6 other peaks to be determined. FIG. 6 shows the detection of glucose, lactose, 6 putative HMOs. Note that the structures of the putative HMOs shown in FIG. 6 are based on the total mass. Detailed structures with specific linkages are determine subsequently.

### Generation of Stable Cell Lines Producing Lactose

Suspension CHO-K1 cells lacking the glutamine synthetase gene (CHO-K1 GS−/−) were generated as previously described (Lin et al 2019, mAbs, 11:5, 965-976). CHO-K1 GS−/− cells were transfected with various DNA constructs containing lactose-expressing genes, as well as the glutamine synthetase selection marker GSR324C, via electroporation using the SG Cell line 4D-Nucleofector® X Kit (LONZA). Briefly, 1×10⁶ cells were transfected with 5 μg of DNA in 100 μl Nucleofector Solution SG. Following transfection, cells were maintained in 50/50 CHO cell medium, a 1:1 ratio mix of PfCho (HyClone) and CD CHO (Thermo Scientific) media, supplemented with L-glutamine (6 mM, Thermo Scientific). At 48 hours post-transfection, the medium was replaced with glutamine-free 50/50 media. Cells were cultured until viability recovered to more than 95% before experimental assays were performed.

### Identification of Human Milk Oligosaccharides (HMOs) Produced by Recombinant Cells

Cells were cultivated in single-use Erlenmeyer flasks (Corning) with a cell seeding density of 0.3×10⁶ cells/mL in 50/50 media. Cell culture supernatant was harvested on day 4 by centrifugation at 200 g for 5 minutes. Clarified supernatant was then applied to Blotglyco beads and columns (BlotGlyco Kit, Shimadzu) for glycan labelling and analysis, according to manufacturer's protocol. Briefly, 160 μL of clarified supernatant was allowed to bind to BlotGlyco beads by incubating for 1 hour at 80° C. until beads were dry. Beads were then washed twice with 2M guanidine hydrochloride, once with distilled water and twice with 1% trimethylamine/methanol solution by centrifugation through a column at 3000 g for 30 seconds. After blocking and wash steps, glycans were released using 2% acetic acid/acetonitrile, and labelled with fluorescent label 2-aminobenzamide (2-AB). 2-AB labelled glycans were washed to remove excess labelling reagent, collected and analyzed with a liquid chromatograph coupled with fluorescence detector and quadruple time-of-flight mass spectrometer (LC-FLD-QTOF). The HMOs attached with 2-AB fluorescence label at the reducing ends enabled detection via fluorescence detector. Prominent peaks from the LC-FLD chromatogram were then tentatively identified based on their respective MS and MS/MS spectra collected with the QTOF mass spectrometer under positive electrospray ionization mode.

### Exemplary Vector Maps and Sequences

Exemplary Vectors 1-9 as disclosed herein are shown in FIG. 8A-FIG. 8C. The recombinant cell as disclosed herein comprises, but not limited to exemplary constructs of the following:
an expression construct having at least 90% or 95% or 100% sequence identity with Vector 1 and an expression construct having at least 90% or 95% or 100% sequence identity with Vector 2; or
an expression construct having at least 90% or 95% or 100% sequence identity with Vector 3 and an expression construct having at least 90% or 95% or 100% sequence identity with Vector 2; or
an expression construct having at least 90% or 95% or 100% sequence identity with Vector 4 and an expression construct having at least 90% or 95% or 100% sequence identity with Vector 2 or
an expression construct having at least 90% or 95% or 100% sequence identity with Vector 5 and an expression construct having at least 90% or 95% or 100% sequence identity with Vector 6 or
an expression construct having at least 90% or 95% or 100% sequence identity with Vector 7 and an expression construct having at least 90% or 95% or 100% sequence identity with Vector 2 or
an expression construct having at least 90% or 95% or 100% sequence identity with Vector 8 and an expression construct having at least 90% or 95% or 100% sequence identity with Vector 2 or
an expression construct having at least 90% or 95% or 100% sequence identity with Vector 9 and an expression construct having at least 90% or 95% or 100% sequence identity with Vector 2 or
an expression construct having at least 90% or 95% or 100% sequence identity with Vector 4.

List of exemplary sequences are listed below:

Homo sapiens lactalbumin alpha (LALBA),
mRNA (SEQ ID NO: 1)
NCBI Reference Sequence: NM_002289.2
>NM_002289.2:27-455 Homo sapiens lactalbumin
alpha (LALBA), mRNA
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGA
Homo sapiens beta-1,4-galactosyltransferase 1
(B4GALT1), transcript variant 1, mRNA
(SEQ ID NO: 2)
NCBI Reference Sequence: NM_001497.4
>NM_001497.4:168-1364 Homo sapiens beta-1,4-
galactosyltransferase 1 (B4GALT1),
transcript variant 1, mRNA
ATGAGGCTTCGGGAGCCGCTCCTGAGCGGCAGCGCCGCGATGCCA
GGCGCGTCCCTACAGCGGGCCTGCCGCCTGCTCGTGGCCGTCTGC
GCTCTGCACCTTGGCGTCACCCTCGTTTACTACCTGGCTGGCCGC
GACCTGAGCCGCCTGCCCCAACTGGTCGGAGTCTCCACACCGCTG
CAGGGCGGCTCGAACAGTGCCGCCGCCATCGGGCAGTCCTCCGGG
GAGCTCCGGACCGGAGGGGCCCGGCCGCCGCCTCCTCTAGGCGCC
TCCTCCCAGCCGCGCCCGGGTGGCGACTCCAGCCCAGTCGTGGAT
TCTGGCCCTGGCCCCGCTAGCAACTTGACCTCGGTCCCAGTGCCC
CACACCACCGCACTGTCGCTGCCCGCCTGCCCTGAGGAGTCCCCG
CTGCTTGTGGGCCCCATGCTGATTGAGTTTAACATGCCTGTGGAC
CTGGAGCTCGTGGCAAAGCAGAACCCAAATGTGAAGATGGGCGGC
CGCTATGCCCCCAGGGACTGCGTCTCTCCTCACAAGGTGGCCATC
ATCATTCCATTCCGCAACCGGCAGGAGCACCTCAAGTACTGGCTA
TATTATTTGCACCCAGTCCTGCAGCGCCAGCAGCTGGACTATGGC
ATCTATGTTATCAACCAGGCGGGAGACACTATATTCAATCGTGCT
AAGCTCCTCAATGTTGGCTTTCAAGAAGCCTTGAAGGACTATGAC
TACACCTGCTTTGTGTTTAGTGACGTGGACCTCATTCCAATGAAT
GACCATAATGCGTACAGGTGTTTTTCACAGCCACGGCACATTTCC
GTTGCAATGGATAAGTTTGGATTCAGCCTACCTTATGTTCAGTAT
TTTGGAGGTGTCTCTGCTCTAAGTAAACAACAGTTTCTAACCATC
AATGGATTTCCTAATAATTATTGGGGCTGGGGAGGAGAAGATGAT
GACATTTTTAACAGATTAGTTTTTAGAGGCATGTCTATATCTCGC
CCAAATGCTGTGGTCGGGAGGTGTCGCATGATCCGCCACTCAAGA
GACAAGAAAAATGAACCCAATCCTCAGAGGTTTGACCGAATTGCA
CACACAAAGGAGACAATGCTCTCTGATGGTTTGAACTCACTCACC
TACCAGGTGCTGGATGTACAGAGATACCCATTGTATACCCAAATC
ACAGTGGACATCGGGACACCGAGCTAG
GLUT1 (full-length) (SEQ ID NO: 3)
Homo sapiens solute carrier family 2 member 1
(SLC2A1), mRNA. NCBI Reference
Sequence: NM_006516.4 >NM_006516.4:
218-1696 Homo sapiens solute carrier family 2
member 1 (SLC2A1), mRNA.
ATGGAGCCCAGCAGCAAGAAGCTGACGGGTCGCCTCATGCTGGCC
GTGGGAGGAGCAGTGCTTGGCTCCCTGCAGTTTGGCTACAACACT
GGAGTCATCAATGCCCCCCAGAAGGTGATCGAGGAGTTCTACAAC
CAGACATGGGTCCACCGCTATGGGGAGAGCATCCTGCCCACCACG
CTCACCACGCTCTGGTCCCTCTCAGTGGCCATCTTTTCTGTTGGG
GGCATGATTGGCTCCTTCTCTGTGGGCCTTTTCGTTAACCGCTTT
GGCCGGCGGAATTCAATGCTGATGATGAACCTGCTGGCCTTCGTG
TCCGCCGTGCTCATGGGCTTCTCGAAACTGGGCAAGTCCTTTGAG
ATGCTGATCCTGGGCCGCTTCATCATCGGTGTGTACTGCGGCCTG
ACCACAGGCTTCGTGCCCATGTATGTGGGTGAAGTGTCACCCACA
GCCCTTCGTGGGGCCCTGGGCACCCTGCACCAGCTGGGCATCGTC
GTCGGCATCCTCATCGCCCAGGTGTTCGGCCTGGACTCCATCATG
GGCAACAAGGACCTGTGGCCCCTGCTGCTGAGCATCATCTTCATC
CCGGCCCTGCTGCAGTGCATCGTGCTGCCCTTCTGCCCCGAGAGT
CCCCGCTTCCTGCTCATCAACCGCAACGAGGAGAACCGGGCCAAG
AGTGTGCTAAAGAAGCTGCGCGGGACAGCTGACGTGACCCATGAC
CTGCAGGAGATGAAGGAAGAGAGTCGGCAGATGATGCGGGAGAAG
AAGGTCACCATCCTGGAGCTGTTCCGCTCCCCCGCCTACCGCCAG
CCCATCCTCATCGCTGTGGTGCTGCAGCTGTCCCAGCAGCTGTCT
GGCATCAACGCTGTCTTCTATTACTCCACGAGCATCTTCGAGAAG
GCGGGGGTGCAGCAGCCTGTGTATGCCACCATTGGCTCCGGTATC
GTCAACACGGCCTTCACTGTCGTGTCGCTGTTTGTGGTGGAGCGA
GCAGGCCGGCGGACCCTGCACCTCATAGGCCTCGCTGGCATGGCG
GGTTGTGCCATACTCATGACCATCGCGCTAGCACTGCTGGAGCAG
CTACCCTGGATGTCCTATCTGAGCATCGTGGCCATCTTTGGCTTT
GTGGCCTTCTTTGAAGTGGGTCCTGGCCCCATCCCATGGTTCATC
GTGGCTGAACTCTTCAGCCAGGGTCCACGTCCAGCTGCCATTGCC
GTTGCAGGCTTCTCCAACTGGACCTCAAATTTCATTGTGGGCATG
TGCTTCCAGTATGTGGAGCAACTGTGTGGTCCCTACGTCTTCATC
ATCTTCACTGTGCTCCTGGTTCTGTTCTTCATCTTCACCTACTTC
AAAGTTCCTGAGACTAAAGGCCGGACCTTCGATGAGATCGCTTCC
GGCTTCCGGCAGGGGGGAGCCAGCCAAAGTGACAAGACACCCGAG
GAGCTGTTCCATCCCCTGGGGGCTGATTCCCAAGTGTGA
GLUT1Δ (cytosolic tail deletion) (SEQ ID NO: 4)
C-terminal truncated version of homo sapiens
solute carrier family 2 member 1 (SLC2A1),
mRNA. NCBI Reference Sequence: NM_006516.4
>NM_006516.4:218-1696 Homo sapiens
solute carrier family 2 member 1 (SLC2A1),
mRNA.
ATGGAGCCCAGCAGCAAGAAGCTGACGGGTCGCCTCATGCTGGCC
GTGGGAGGAGCAGTGCTTGGCTCCCTGCAGTTTGGCTACAACACT
GGAGTCATCAATGCCCCCCAGAAGGTGATCGAGGAGTTCTACAAC
CAGACATGGGTCCACCGCTATGGGGAGAGCATCCTGCCCACCACG
CTCACCACGCTCTGGTCCCTCTCAGTGGCCATCTTTTCTGTTGGG
GGCATGATTGGCTCCTTCTCTGTGGGCCTTTTCGTTAACCGCTTT
GGCCGGCGGAATTCAATGCTGATGATGAACCTGCTGGCCTTCGTG
TCCGCCGTGCTCATGGGCTTCTCGAAACTGGGCAAGTCCTTTGAG
ATGCTGATCCTGGGCCGCTTCATCATCGGTGTGTACTGCGGCCTG
ACCACAGGCTTCGTGCCCATGTATGTGGGTGAAGTGTCACCCACA
GCCCTTCGTGGGGCCCTGGGCACCCTGCACCAGCTGGGCATCGTC
GTCGGCATCCTCATCGCCCAGGTGTTCGGCCTGGACTCCATCATG
GGCAACAAGGACCTGTGGCCCCTGCTGCTGAGCATCATCTTCATC
CCGGCCCTGCTGCAGTGCATCGTGCTGCCCTTCTGCCCCGAGAGT
CCCCGCTTCCTGCTCATCAACCGCAACGAGGAGAACCGGGCCAAG
AGTGTGCTAAAGAAGCTGCGCGGGACAGCTGACGTGACCCATGAC
CTGCAGGAGATGAAGGAAGAGAGTCGGCAGATGATGCGGGAGAAG
AAGGTCACCATCCTGGAGCTGTTCCGCTCCCCCGCCTACCGCCAG
CCCATCCTCATCGCTGTGGTGCTGCAGCTGTCCCAGCAGCTGTCT
GGCATCAACGCTGTCTTCTATTACTCCACGAGCATCTTCGAGAAG
GCGGGGGTGCAGCAGCCTGTGTATGCCACCATTGGCTCCGGTATC
GTCAACACGGCCTTCACTGTCGTGTCGCTGTTTGTGGTGGAGCGA
GCAGGCCGGCGGACCCTGCACCTCATAGGCCTCGCTGGCATGGCG
GGTTGTGCCATACTCATGACCATCGCGCTAGCACTGCTGGAGCAG
CTACCCTGGATGTCCTATCTGAGCATCGTGGCCATCTTTGGCTTT
GTGGCCTTCTTTGAAGTGGGTCCTGGCCCCATCCCATGGTTCATC
GTGGCTGAACTCTTCAGCCAGGGTCCACGTCCAGCTGCCATTGCC
GTTGCAGGCTTCTCCAACTGGACCTCAAATTTCATTGTGGGCATG
TGCTTCCAGTATGTGGAGCAACTGTGTGGTCCCTACGTCTTCATC
ATCTTCACTGTGCTCCTGGTTCTGTTCTTCATCTTCACCTACTTC
eGFP sequence (SEQ ID NO: 5)
ATGGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGGACGGCAGCGTGCAGCTCGCC
GACCACTACCAGCAGAACACCCCCATCGGCGACGGCCCCGTGCTG
CTGCCCGACAACCACTACCTGAGCACCCAGTCCGCCCTGAGCAAA
GACCCCAACGAGAAGCGCGATCACATGGTCCTGCTGGAGTTCGTG
ACCGCCGCCGGGATCACTCTCGGCATGGACGAGCTGTACAAGTAA
IRES2 (SEQ ID NO: 6)
CCCCTCTCCCTCCCCCCCCCCTAACGTTACTGGCCGAAGCCGCTT
GGAATAAGGCCGGTGTGCGTTTGTCTATATGTTATTTTCCACCAT
ATTGCCGTCTTTTGGCAATGTGAGGGCCCGGAAACCTGGCCCTGT
CTTCTTGACGAGCATTCCTAGGGGTCTTTCCCCTCTCGCCAAAGG
AATGCAAGGTCTGTTGAATGTCGTGAAGGAAGCAGTTCCTCTGGA
AGCTTCTTGAAGACAAACAACGTCTGTAGCGACCCTTTGCAGGCA
GCGGAACCCCCCACCTGGCGACAGGTGCCTCTGCGGCCAAAAGCC
ACGTGTATAAGATACACCTGCAAAGGCGGCACAACCCCAGTGCCA
CGTTGTGAGTTGGATAGTTGTGGAAAGAGTCAAATGGCTCTCCTC
AAGCGTATTCAACAAGGGGCTGAAGGATGCCCAGAAGGTACCCCA
TTGTATGGGATCTGATCTGGGGCCTCGGTACACATGCTTTACATG
TGTTTAGTCGAGGTTAAAAAAACGTCTAGGCCCCCCGAACCACGG
GGACGTGGTTTTCCTTTGAAAAACACGATGATAATATGGCCACAA
CC
Glutamine Synthetase (GS) (SEQ ID NO: 7)
ATGGCCACCTCAGCAAGTTCCCACTTGAACAAAAACATCAAGCAA
ATGTACTTGTGCCTGCCCCAGGGTGAGAAAGTCCAAGCCATGTAT
ATCTGGGTTGATGGTACTGGAGAAGGACTGCGCTGCAAAACCCGC
ACCCTGGACTGTGAGCCCAAGTGTGTAGAAGAGTTACCTGAGTGG
AATTTTGATGGCTCTAGTACCTTTCAGTCTGAGGGCTCCAACAGT
GACATGTATCTCAGCCCTGTTGCCATGTTTCGGGACCCCTTCCGC
AGAGATCCCAACAAGCTGGTGTTCTGTGAAGTTTTCAAGTACAAC
CGGAAGCCTGCAGAGACCAATTTAAGGCACTCGTGTAAACGGATA
ATGGACATGGTGAGCAACCAGCACCCCTGGTTTGGAATGGAACAG
GAGTATACTCTGATGGGAACAGATGGGCACCCTTTTGGTTGGCCT
TCCAATGGCTTTCCTGGGCCCCAAGGTCCGTATTACTGTGGTGTG
GGCGCAGACAAAGCCTATGGCAGGGATATCGTGGAGGCTCACTAC
CGCGCCTGCTTGTATGCTGGGGTCAAGATTACAGGAACAAATGCT
GAGGTCATGCCTGCCCAGTGGGAATTCCAAATAGGACCCTGTGAA
GGAATCCGCATGGGAGATCATCTCTGGGTGGCCCGTTTCATCTTG
CATCGAGTATGTGAAGACTTTGGGGTAATAGCAACCTTTGACCCC
AAGCCCATTCCTGGGAACTGGAATGGTGCAGGCTGCCATACCAAC
TTTAGCACCAAGGCCATGCGGGAGGAGAATGGTCTGAAGCACATC
GAGGAGGCCATCGAGAAACTAAGCAAGCGGCACCGGTACCACATT
CGAGCCTACGATCCCAAGGGGGGCCTGGACAATGCCCGTCGTCTG
ACTGGGTTCCACGAAACGTCCAACATCAACGACTTTTCTGCTGGT
GTCGCCAATCGCAGTGCCAGCATCTGCATTCCCCGGACTGTCGGC
CAGGAGAAGAAAGGTTACTTTGAAGACCGCCGCCCCTCTGCCAAT
TGTGACCCCTTTGCAGTGACAGAAGCCATCGTCCGCACATGCCTT
CTCAATGAGACTGGCGACGAGCCCTTCCAATACAAAAACTAA
Bleomycin resistance protein (BleoR)
(SEQ ID NO: 8)
ATGGCCAAGTTGACCAGTGCCGTTCCGGTGCTCACCGCGCGCGAC
GTCGCCGGAGCGGTCGAGTTCTGGACCGACCGGCTCGGGTTCTCC
CGGGACTTCGTGGAGGACGACTTCGCCGGTGTGGTCCGGGACGAC
GTGACCCTGTTCATCAGCGCGGTCCAGGACCAGGTGGTGCCGGAC
AACACCCTGGCCTGGGTGTGGGTGCGCGGCCTGGACGAGCTGTAC
GCCGAGTGGTCGGAGGTCGTGTCCACGAACTTCCGGGACGCCTCC
GGGCCGGCCATGACCGAGATCGGCGAGCAGCCGTGGGGGGGGGAG
TTCGCCCTGCGCGACCCGGCCGGCAACTGCGTGCACTTCGTGGCC
GAGGAGCAGGACTGA
Homo sapiens solute carrier family 35
(CMP-sialic acid transporter), member A1,
mRNA (cDNA clone MGC: 22375 IMAGE: 4690513),
complete cds (SEQ ID NO: 9)
GenBank: BC017807.1
ATGGCTGCCCCGAGAGACAATGTCACTTTATTATTCAAGTTATAC
TGCTTGGCAGTGATGACCCTGATGGCTGCAGTCTATACCATAGCT
TTAAGATACACAAGGACATCAGACAAAGAACTCTACTTTTCAACC
ACAGCCGTGTGTATCACAGAAGTTATAAAGTTATTGCTAAGTGTG
GGAATTTTAGCTAAAGAAACTGGTAGTCTGGGTAGATTCAAAGCA
TCTTTAAGAGAAAATGTCTTGGGGAGCCCCAAGGAACTGTTGAAG
TTAAGTGTGCCATCGTTAGTGTATGCTGTTCAGAACAACATGGCT
TTCCTAGCTCTTAGCAATCTGGATGCAGCAGTGTACCAGGTGACC
TACCAGTTGAAGATTCCGTGTACTGCTTTATGCACTGTTTTAATG
TTAAACCGGACACTCAGCAAATTACAGTGGGTTTCAGTTTTTATG
CTGTGTGCTGGAGTTACGCTTGTACAGTGGAAACCAGCCCAAGCT
ACAAAAGTGGTGGTGGAACAAAATCCATTATTAGGGTTTGGCGCT
ATAGCTATTGCTGTATTGTGCTCAGGATTTGCAGGAGTATATTTT
GAAAAAGTTTTAAAGAGTTCAGATACTTCTCTTTGGGTGAGAAAC
ATTCAAATGTATCTATCAGGGATTATTGTGACATTAGCTGGCGTC
TACTTGTCAGATGGAGCTGAAATTAAAGAAAAAGGATTTTTCTAT
GGTTACACATATTATGTCTGGTTTGTCATCTTTCTTGCAAGTGTT
GGTGGCCTCTACACTTCTGTTGTGGTTAAGTACACAGACAACATC
ATGAAAGGCTTTTCTGCAGCAGCGGCCATTGTCCTTTCCACCATT
GCTTCAGTAATGCTGTTTGGATTACAGATAACACTCACCTTTGCC
CTGGGTACTCTTCTTGTATGTGTTTCCATATATCTCTATGGATTA
CCCAGACAAGACACTACATCCATCCAACAAGGAGAAACAGCTTCA
AAGGAGAGAGTTATTGGTGTGTGA
ER localization sequence: (SEQ ID NO: 10)
CTGCTGACCAAGGTGAAGGGCTCC
Golgi localization sequence: (SEQ ID NO: 11)
CCCAGACAAGACACTACATCCATCCAACAAGGAGAAACAGCTTCA
AAGGAGAGAGTTATTGGTGTG
Construct set 1: hLALBA-B4GALT1-hGLUT1-GFP(CT)
Sequence 1 (SEQ ID NO: 12):
hLALBA(SEQ ID NO: 1)-CTCGAG(linker)-
IRES2 (SEQ ID NO: 6)-B4GALT1(SEQ ID NO: 2)-
CTCGAG(linker)-IRES2(SEQ ID NO: 6)-
GS(SEQ ID NO: 7)
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGAGGATCCGCCCCTCTCCCTCCC
CCCCCCCTAACGTTACTGGCCGAAGCCGCTTGGAATAAGGCCGGT
GTGCGTTTGTCTATATGTTATTTTCCACCATATTGCCGTCTTTTG
GCAATGTGAGGGCCCGGAAACCTGGCCCTGTCTTCTTGACGAGCA
TTCCTAGGGGTCTTTCCCCTCTCGCCAAAGGAATGCAAGGTCTGT
TGAATGTCGTGAAGGAAGCAGTTCCTCTGGAAGCTTCTTGAAGAC
AAACAACGTCTGTAGCGACCCTTTGCAGGCAGCGGAACCCCCCAC
CTGGCGACAGGTGCCTCTGCGGCCAAAAGCCACGTGTATAAGATA
CACCTGCAAAGGCGGCACAACCCCAGTGCCACGTTGTGAGTTGGA
TAGTTGTGGAAAGAGTCAAATGGCTCTCCTCAAGCGTATTCAACA
AGGGGCTGAAGGATGCCCAGAAGGTACCCCATTGTATGGGATCTG
ATCTGGGGCCTCGGTACACATGCTTTACATGTGTTTAGTCGAGGT
TAAAAAAACGTCTAGGCCCCCCGAACCACGGGGACGTGGTTTTCC
TTTGAAAAACACGATGATAATATGGCCACAACCATGAGGCTTCGG
GAGCCGCTCCTGAGCGGCAGCGCCGCGATGCCAGGCGCGTCCCTA
CAGCGGGCCTGCCGCCTGCTCGTGGCCGTCTGCGCTCTGCACCTT
GGCGTCACCCTCGTTTACTACCTGGCTGGCCGCGACCTGAGCCGC
CTGCCCCAACTGGTCGGAGTCTCCACACCGCTGCAGGGCGGCTCG
AACAGTGCCGCCGCCATCGGGCAGTCCTCCGGGGAGCTCCGGACC
GGAGGGGCCCGGCCGCCGCCTCCTCTAGGCGCCTCCTCCCAGCCG
CGCCCGGGTGGCGACTCCAGCCCAGTCGTGGATTCTGGCCCTGGC
CCCGCTAGCAACTTGACCTCGGTCCCAGTGCCCCACACCACCGCA
CTGTCGCTGCCCGCCTGCCCTGAGGAGTCCCCGCTGCTTGTGGGC
CCCATGCTGATTGAGTTTAACATGCCTGTGGACCTGGAGCTCGTG
GCAAAGCAGAACCCAAATGTGAAGATGGGCGGCCGCTATGCCCCC
AGGGACTGCGTCTCTCCTCACAAGGTGGCCATCATCATTCCATTC
CGCAACCGGCAGGAGCACCTCAAGTACTGGCTATATTATTTGCAC
CCAGTCCTGCAGCGCCAGCAGCTGGACTATGGCATCTATGTTATC
AACCAGGCGGGAGACACTATATTCAATCGTGCTAAGCTCCTCAAT
GTTGGCTTTCAAGAAGCCTTGAAGGACTATGACTACACCTGCTTT
GTGTTTAGTGACGTGGACCTCATTCCAATGAATGACCATAATGCG
TACAGGTGTTTTTCACAGCCACGGCACATTTCCGTTGCAATGGAT
AAGTTTGGATTCAGCCTACCTTATGTTCAGTATTTTGGAGGTGTC
TCTGCTCTAAGTAAACAACAGTTTCTAACCATCAATGGATTTCCT
AATAATTATTGGGGCTGGGGAGGAGAAGATGATGACATTTTTAAC
AGATTAGTTTTTAGAGGCATGTCTATATCTCGCCCAAATGCTGTG
GTCGGGAGGTGTCGCATGATCCGCCACTCAAGAGACAAGAAAAAT
GAACCCAATCCTCAGAGGTTTGACCGAATTGCACACACAAAGGAG
ACAATGCTCTCTGATGGTTTGAACTCACTCACCTACCAGGTGCTG
GATGTACAGAGATACCCATTGTATACCCAAATCACAGTGGACATC
GGGACACCGAGCTAGCTCGAGCCCCTCTCCCTCCCCCCCCCCTAA
CGTTACTGGCCGAAGCCGCTTGGAATAAGGCCGGTGTGCGTTTGT
CTATATGTTATTTTCCACCATATTGCCGTCTTTTGGCAATGTGAG
GGCCCGGAAACCTGGCCCTGTCTTCTTGACGAGCATTCCTAGGGG
TCTTTCCCCTCTCGCCAAAGGAATGCAAGGTCTGTTGAATGTCGT
GAAGGAAGCAGTTCCTCTGGAAGCTTCTTGAAGACAAACAACGTC
TGTAGCGACCCTTTGCAGGCAGCGGAACCCCCCACCTGGCGACAG
GTGCCTCTGCGGCCAAAAGCCACGTGTATAAGATACACCTGCAAA
GGCGGCACAACCCCAGTGCCACGTTGTGAGTTGGATAGTTGTGGA
AAGAGTCAAATGGCTCTCCTCAAGCGTATTCAACAAGGGGCTGAA
GGATGCCCAGAAGGTACCCCATTGTATGGGATCTGATCTGGGGCC
TCGGTACACATGCTTTACATGTGTTTAGTCGAGGTTAAAAAAACG
TCTAGGCCCCCCGAACCACGGGGACGTGGTTTTCCTTTGAAAAAC
ACGATGATAATATGGCCACAACCATGGCCACCTCAGCAAGTTCCC
ACTTGAACAAAAACATCAAGCAAATGTACTTGTGCCTGCCCCAGG
GTGAGAAAGTCCAAGCCATGTATATCTGGGTTGATGGTACTGGAG
AAGGACTGCGCTGCAAAACCCGCACCCTGGACTGTGAGCCCAAGT
GTGTAGAAGAGTTACCTGAGTGGAATTTTGATGGCTCTAGTACCT
TTCAGTCTGAGGGCTCCAACAGTGACATGTATCTCAGCCCTGTTG
CCATGTTTCGGGACCCCTTCCGCAGAGATCCCAACAAGCTGGTGT
TCTGTGAAGTTTTCAAGTACAACCGGAAGCCTGCAGAGACCAATT
TAAGGCACTCGTGTAAACGGATAATGGACATGGTGAGCAACCAGC
ACCCCTGGTTTGGAATGGAACAGGAGTATACTCTGATGGGAACAG
ATGGGCACCCTTTTGGTTGGCCTTCCAATGGCTTTCCTGGGCCCC
AAGGTCCGTATTACTGTGGTGTGGGCGCAGACAAAGCCTATGGCA
GGGATATCGTGGAGGCTCACTACCGCGCCTGCTTGTATGCTGGGG
TCAAGATTACAGGAACAAATGCTGAGGTCATGCCTGCCCAGTGGG
AATTCCAAATAGGACCCTGTGAAGGAATCCGCATGGGAGATCATC
TCTGGGTGGCCCGTTTCATCTTGCATCGAGTATGTGAAGACTTTG
GGGTAATAGCAACCTTTGACCCCAAGCCCATTCCTGGGAACTGGA
ATGGTGCAGGCTGCCATACCAACTTTAGCACCAAGGCCATGCGGG
AGGAGAATGGTCTGAAGCACATCGAGGAGGCCATCGAGAAACTAA
GCAAGCGGCACCGGTACCACATTCGAGCCTACGATCCCAAGGGGG
GCCTGGACAATGCCCGTCGTCTGACTGGGTTCCACGAAACGTCCA
ACATCAACGACTTTTCTGCTGGTGTCGCCAATCGCAGTGCCAGCA
TCTGCATTCCCCGGACTGTCGGCCAGGAGAAGAAAGGTTACTTTG
AAGACCGCCGCCCCTCTGCCAATTGTGACCCCTTTGCAGTGACAG
AAGCCATCGTCCGCACATGCCTTCTCAATGAGACTGGCGACGAGC
CCTTCCAATACAAAAACTAA
Sequence 2 (SEQ ID NO: 13):
hGLUT1 (SEQ ID NO: 3)-Egfp (SEQ ID NO: 5)-
GGCGCGCC(linker)-IRES2 (SEQ ID NO: 6)-
BleoR (SEQ ID NO: 8)
ATGGAGCCCAGCAGCAAGAAGCTGACGGGTCGCCTCATGCTGGCC
GTGGGAGGAGCAGTGCTTGGCTCCCTGCAGTTTGGCTACAACACT
GGAGTCATCAATGCCCCCCAGAAGGTGATCGAGGAGTTCTACAAC
CAGACATGGGTCCACCGCTATGGGGAGAGCATCCTGCCCACCACG
CTCACCACGCTCTGGTCCCTCTCAGTGGCCATCTTTTCTGTTGGG
GGCATGATTGGCTCCTTCTCTGTGGGCCTTTTCGTTAACCGCTTT
GGCCGGCGGAATTCAATGCTGATGATGAACCTGCTGGCCTTCGTG
TCCGCCGTGCTCATGGGCTTCTCGAAACTGGGCAAGTCCTTTGAG
ATGCTGATCCTGGGCCGCTTCATCATCGGTGTGTACTGCGGCCTG
ACCACAGGCTTCGTGCCCATGTATGTGGGTGAAGTGTCACCCACA
GCCCTTCGTGGGGCCCTGGGCACCCTGCACCAGCTGGGCATCGTC
GTCGGCATCCTCATCGCCCAGGTGTTCGGCCTGGACTCCATCATG
GGCAACAAGGACCTGTGGCCCCTGCTGCTGAGCATCATCTTCATC
CCGGCCCTGCTGCAGTGCATCGTGCTGCCCTTCTGCCCCGAGAGT
CCCCGCTTCCTGCTCATCAACCGCAACGAGGAGAACCGGGCCAAG
AGTGTGCTAAAGAAGCTGCGCGGGACAGCTGACGTGACCCATGAC
CTGCAGGAGATGAAGGAAGAGAGTCGGCAGATGATGCGGGAGAAG
AAGGTCACCATCCTGGAGCTGTTCCGCTCCCCCGCCTACCGCCAG
CCCATCCTCATCGCTGTGGTGCTGCAGCTGTCCCAGCAGCTGTCT
GGCATCAACGCTGTCTTCTATTACTCCACGAGCATCTTCGAGAAG
GCGGGGGTGCAGCAGCCTGTGTATGCCACCATTGGCTCCGGTATC
GTCAACACGGCCTTCACTGTCGTGTCGCTGTTTGTGGTGGAGCGA
GCAGGCCGGCGGACCCTGCACCTCATAGGCCTCGCTGGCATGGCG
GGTTGTGCCATACTCATGACCATCGCGCTAGCACTGCTGGAGCAG
CTACCCTGGATGTCCTATCTGAGCATCGTGGCCATCTTTGGCTTT
GTGGCCTTCTTTGAAGTGGGTCCTGGCCCCATCCCATGGTTCATC
GTGGCTGAACTCTTCAGCCAGGGTCCACGTCCAGCTGCCATTGCC
GTTGCAGGCTTCTCCAACTGGACCTCAAATTTCATTGTGGGCATG
TGCTTCCAGTATGTGGAGCAACTGTGTGGTCCCTACGTCTTCATC
ATCTTCACTGTGCTCCTGGTTCTGTTCTTCATCTTCACCTACTTC
AAAGTTCCTGAGACTAAAGGCCGGACCTTCGATGAGATCGCTTCC
GGCTTCCGGCAGGGGGGAGCCAGCCAAAGTGACAAGACACCCGAG
GAGCTGTTCCATCCCCTGGGGGCTGATTCCCAAGTGGTGAGCAAG
GGCGAGGAGCTGTTCACCGGGGTGGTGCCCATCCTGGTCGAGCTG
GACGGCGACGTAAACGGCCACAAGTTCAGCGTGTCCGGCGAGGGC
GAGGGCGATGCCACCTACGGCAAGCTGACCCTGAAGTTCATCTGC
ACCACCGGCAAGCTGCCCGTGCCCTGGCCCACCCTCGTGACCACC
CTGACCTACGGCGTGCAGTGCTTCAGCCGCTACCCCGACCACATG
AAGCAGCACGACTTCTTCAAGTCCGCCATGCCCGAAGGCTACGTC
CAGGAGCGCACCATCTTCTTCAAGGACGACGGCAACTACAAGACC
CGCGCCGAGGTGAAGTTCGAGGGCGACACCCTGGTGAACCGCATC
GAGCTGAAGGGCATCGACTTCAAGGAGGACGGCAACATCCTGGGG
CACAAGCTGGAGTACAACTACAACAGCCACAACGTCTATATCATG
GCCGACAAGCAGAAGAACGGCATCAAGGTGAACTTCAAGATCCGC
CACAACATCGAGGACGGCAGCGTGCAGCTCGCCGACCACTACCAG
CAGAACACCCCCATCGGCGACGGCCCCGTGCTGCTGCCCGACAAC
CACTACCTGAGCACCCAGTCCGCCCTGAGCAAAGACCCCAACGAG
AAGCGCGATCACATGGTCCTGCTGGAGTTCGTGACCGCCGCCGGG
ATCACTCTCGGCATGGACGAGCTGTACAAGTAAGGCGCGCCCCCC
TCTCCCTCCCCCCCCCCTAACGTTACTGGCCGAAGCCGCTTGGAA
TAAGGCCGGTGTGCGTTTGTCTATATGTTATTTTCCACCATATTG
CCGTCTTTTGGCAATGTGAGGGCCCGGAAACCTGGCCCTGTCTTC
TTGACGAGCATTCCTAGGGGTCTTTCCCCTCTCGCCAAAGGAATG
CAAGGTCTGTTGAATGTCGTGAAGGAAGCAGTTCCTCTGGAAGCT
TCTTGAAGACAAACAACGTCTGTAGCGACCCTTTGCAGGCAGCGG
AACCCCCCACCTGGCGACAGGTGCCTCTGCGGCCAAAAGCCACGT
GTATAAGATACACCTGCAAAGGCGGCACAACCCCAGTGCCACGTT
GTGAGTTGGATAGTTGTGGAAAGAGTCAAATGGCTCTCCTCAAGC
GTATTCAACAAGGGGCTGAAGGATGCCCAGAAGGTACCCCATTGT
ATGGGATCTGATCTGGGGCCTCGGTACACATGCTTTACATGTGTT
TAGTCGAGGTTAAAAAAACGTCTAGGCCCCCCGAACCACGGGGAC
GTGGTTTTCCTTTGAAAAACACGATGATAATATGGCCACAACCAT
GGCCAAGTTGACCAGTGCCGTTCCGGTGCTCACCGCGCGCGACGT
CGCCGGAGCGGTCGAGTTCTGGACCGACCGGCTCGGGTTCTCCCG
GGACTTCGTGGAGGACGACTTCGCCGGTGTGGTCCGGGACGACGT
GACCCTGTTCATCAGCGCGGTCCAGGACCAGGTGGTGCCGGACAA
CACCCTGGCCTGGGTGTGGGTGCGCGGCCTGGACGAGCTGTACGC
CGAGTGGTCGGAGGTCGTGTCCACGAACTTCCGGGACGCCTCCGG
GCCGGCCATGACCGAGATCGGCGAGCAGCCGTGGGGGGGGAGTTC
GCCCTGCGCGACCCGGCCGGCAACTGCGTGCACTTCGTGGCCGAG
GAGCAGGACTGA
Construct set 2: Hb4GALT1-Hlalba
Sequence 1 (SEQ ID NO: 14):
hB4GALT1 (SEQ ID NO: 2)-IRES2(SEQ ID NO: 6)-
hLALBA (SEQ ID NO: 1)-CTCGAG(linker)-
IRES2 (SEQ ID NO: 6)-GS (SEQ ID NO: 7)
ATGAGGCTTCGGGAGCCGCTCCTGAGCGGCAGCGCCGCGATGCCA
GGCGCGTCCCTACAGCGGGCCTGCCGCCTGCTCGTGGCCGTCTGC
GCTCTGCACCTTGGCGTCACCCTCGTTTACTACCTGGCTGGCCGC
GACCTGAGCCGCCTGCCCCAACTGGTCGGAGTCTCCACACCGCTG
CAGGGCGGCTCGAACAGTGCCGCCGCCATCGGGCAGTCCTCCGGG
GAGCTCCGGACCGGAGGGGCCCGGCCGCCGCCTCCTCTAGGCGCC
TCCTCCCAGCCGCGCCCGGGTGGCGACTCCAGCCCAGTCGTGGAT
TCTGGCCCTGGCCCCGCTAGCAACTTGACCTCGGTCCCAGTGCCC
CACACCACCGCACTGTCGCTGCCCGCCTGCCCTGAGGAGTCCCCG
CTGCTTGTGGGCCCCATGCTGATTGAGTTTAACATGCCTGTGGAC
CTGGAGCTCGTGGCAAAGCAGAACCCAAATGTGAAGATGGGCGGC
CGCTATGCCCCCAGGGACTGCGTCTCTCCTCACAAGGTGGCCATC
ATCATTCCATTCCGCAACCGGCAGGAGCACCTCAAGTACTGGCTA
TATTATTTGCACCCAGTCCTGCAGCGCCAGCAGCTGGACTATGGC
ATCTATGTTATCAACCAGGCGGGAGACACTATATTCAATCGTGCT
AAGCTCCTCAATGTTGGCTTTCAAGAAGCCTTGAAGGACTATGAC
TACACCTGCTTTGTGTTTAGTGACGTGGACCTCATTCCAATGAAT
GACCATAATGCGTACAGGTGTTTTTCACAGCCACGGCACATTTCC
GTTGCAATGGATAAGTTTGGATTCAGCCTACCTTATGTTCAGTAT
TTTGGAGGTGTCTCTGCTCTAAGTAAACAACAGTTTCTAACCATC
AATGGATTTCCTAATAATTATTGGGGCTGGGGAGGAGAAGATGAT
GACATTTTTAACAGATTAGTTTTTAGAGGCATGTCTATATCTCGC
CCAAATGCTGTGGTCGGGAGGTGTCGCATGATCCGCCACTCAAGA
GACAAGAAAAATGAACCCAATCCTCAGAGGTTTGACCGAATTGCA
CACACAAAGGAGACAATGCTCTCTGATGGTTTGAACTCACTCACC
TACCAGGTGCTGGATGTACAGAGATACCCATTGTATACCCAAATC
ACAGTGGACATCGGGACACCGAGCTAGGGATCCCCTCTCCCTCCC
CCCCCCCTAACGTTACTGGCCGAAGCCGCTTGGAATAAGGCCGGT
GTGCGTTTGTCTATATGTTATTTTCCACCATATTGCCGTCTTTTG
GCAATGTGAGGGCCCGGAAACCTGGCCCTGTCTTCTTGACGAGCA
TTCCTAGGGGTCTTTCCCCTCTCGCCAAAGGAATGCAAGGTCTGT
TGAATGTCGTGAAGGAAGCAGTTCCTCTGGAAGCTTCTTGAAGAC
AAACAACGTCTGTAGCGACCCTTTGCAGGCAGCGGAACCCCCCAC
CTGGCGACAGGTGCCTCTGCGGCCAAAAGCCACGTGTATAAGATA
CACCTGCAAAGGCGGCACAACCCCAGTGCCACGTTGTGAGTTGGA
TAGTTGTGGAAAGAGTCAAATGGCTCTCCTCAAGCGTATTCAACA
AGGGGCTGAAGGATGCCCAGAAGGTACCCCATTGTATGGGATCTG
ATCTGGGGCCTCGGTACACATGCTTTACATGTGTTTAGTCGAGGT
TAAAAAAACGTCTAGGCCCCCCGAACCACGGGGACGTGGTTTTCC
TTTGAAAAACACGATGATAATATGGCCACAACCATGAGGTTCTTT
GTCCCTCTGTTCCTGGTGGGCATCCTGTTCCCTGCCATCCTGGCC
AAGCAATTCACAAAATGTGAGCTGTCCCAGCTGCTGAAAGACATA
GATGGTTATGGAGGCATCGCTTTGCCTGAATTGATCTGTACCATG
TTTCACACCAGTGGTTATGACACACAAGCCATAGTTGAAAACAAT
GAAAGCACGGAATATGGACTCTTCCAGATCAGTAATAAGCTTTGG
TGCAAGAGCAGCCAGGTCCCTCAGTCAAGGAACATCTGTGACATC
TCCTGTGACAAGTTCCTGGATGATGACATTACTGATGACATAATG
TGTGCCAAGAAGATCCTGGATATTAAAGGAATTGACTACTGGTTG
GCCCATAAAGCCCTCTGCACTGAGAAGCTGGAACAGTGGCTTTGT
GAGAAGTTGTGActcgagCCCCTCTCCCTCCCCCCCCCCTAACGT
TACTGGCCGAAGCCGCTTGGAATAAGGCCGGTGTGCGTTTGTCTA
TATGTTATTTTCCACCATATTGCCGTCTTTTGGCAATGTGAGGGC
CCGGAAACCTGGCCCTGTCTTCTTGACGAGCATTCCTAGGGGTCT
TTCCCCTCTCGCCAAAGGAATGCAAGGTCTGTTGAATGTCGTGAA
GGAAGCAGTTCCTCTGGAAGCTTCTTGAAGACAAACAACGTCTGT
AGCGACCCTTTGCAGGCAGCGGAACCCCCCACCTGGCGACAGGTG
CCTCTGCGGCCAAAAGCCACGTGTATAAGATACACCTGCAAAGGC
GGCACAACCCCAGTGCCACGTTGTGAGTTGGATAGTTGTGGAAAG
AGTCAAATGGCTCTCCTCAAGCGTATTCAACAAGGGGCTGAAGGA
TGCCCAGAAGGTACCCCATTGTATGGGATCTGATCTGGGGCCTCG
GTACACATGCTTTACATGTGTTTAGTCGAGGTTAAAAAAACGTCT
AGGCCCCCCGAACCACGGGGACGTGGTTTTCCTTTGAAAAACACG
ATGATAATATGGCCACAACCATGGCCACCTCAGCAAGTTCCCACT
TGAACAAAAACATCAAGCAAATGTACTTGTGCCTGCCCCAGGGTG
AGAAAGTCCAAGCCATGTATATCTGGGTTGATGGTACTGGAGAAG
GACTGCGCTGCAAAACCCGCACCCTGGACTGTGAGCCCAAGTGTG
TAGAAGAGTTACCTGAGTGGAATTTTGATGGCTCTAGTACCTTTC
AGTCTGAGGGCTCCAACAGTGACATGTATCTCAGCCCTGTTGCCA
TGTTTCGGGACCCCTTCCGCAGAGATCCCAACAAGCTGGTGTTCT
GTGAAGTTTTCAAGTACAACCGGAAGCCTGCAGAGACCAATTTAA
GGCACTCGTGTAAACGGATAATGGACATGGTGAGCAACCAGCACC
CCTGGTTTGGAATGGAACAGGAGTATACTCTGATGGGAACAGATG
GGCACCCTTTTGGTTGGCCTTCCAATGGCTTTCCTGGGCCCCAAG
GTCCGTATTACTGTGGTGTGGGCGCAGACAAAGCCTATGGCAGGG
ATATCGTGGAGGCTCACTACCGCGCCTGCTTGTATGCTGGGGTCA
AGATTACAGGAACAAATGCTGAGGTCATGCCTGCCCAGTGGGAAT
TCCAAATAGGACCCTGTGAAGGAATCCGCATGGGAGATCATCTCT
GGGTGGCCCGTTTCATCTTGCATCGAGTATGTGAAGACTTTGGGG
TAATAGCAACCTTTGACCCCAAGCCCATTCCTGGGAACTGGAATG
GTGCAGGCTGCCATACCAACTTTAGCACCAAGGCCATGCGGGAGG
AGAATGGTCTGAAGCACATCGAGGAGGCCATCGAGAAACTAAGCA
AGCGGCACCGGTACCACATTCGAGCCTACGATCCCAAGGGGGGCC
TGGACAATGCCCGTCGTCTGACTGGGTTCCACGAAACGTCCAACA
TCAACGACTTTTCTGCTGGTGTCGCCAATCGCAGTGCCAGCATCT
GCATTCCCCGGACTGTCGGCCAGGAGAAGAAAGGTTACTTTGAAG
ACCGCCGCCCCTCTGCCAATTGTGACCCCTTTGCAGTGACAGAAG
CCATCGTCCGCACATGCCTTCTCAATGAGACTGGCGACGAGCCCT
TCCAATACAAAAACTAA
Sequence 2 (SEQ ID NO: 15):
IRES2 (SEQ ID NO: 6)-BleoR (SEQ ID NO: 8)
CCCCTCTCCCTCCCCCCCCCCTAACGTTACTGGCCGAAGCCGCTT
GGAATAAGGCCGGTGTGCGTTTGTCTATATGTTATTTTCCACCAT
ATTGCCGTCTTTTGGCAATGTGAGGGCCCGGAAACCTGGCCCTGT
CTTCTTGACGAGCATTCCTAGGGGTCTTTCCCCTCTCGCCAAAGG
AATGCAAGGTCTGTTGAATGTCGTGAAGGAAGCAGTTCCTCTGGA
AGCTTCTTGAAGACAAACAACGTCTGTAGCGACCCTTTGCAGGCA
GCGGAACCCCCCACCTGGCGACAGGTGCCTCTGCGGCCAAAAGCC
ACGTGTATAAGATACACCTGCAAAGGCGGCACAACCCCAGTGCCA
CGTTGTGAGTTGGATAGTTGTGGAAAGAGTCAAATGGCTCTCCTC
AAGCGTATTCAACAAGGGGCTGAAGGATGCCCAGAAGGTACCCCA
TTGTATGGGATCTGATCTGGGGCCTCGGTACACATGCTTTACATG
TGTTTAGTCGAGGTTAAAAAAACGTCTAGGCCCCCCGAACCACGG
GGACGTGGTTTTCCTTTGAAAAACACGATGATAATATGGCCACAA
CCATGGCCAAGTTGACCAGTGCCGTTCCGGTGCTCACCGCGCGCG
ACGTCGCCGGAGCGGTCGAGTTCTGGACCGACCGGCTCGGGTTCT
CCCGGGACTTCGTGGAGGACGACTTCGCCGGTGTGGTCCGGGACG
ACGTGACCCTGTTCATCAGCGCGGTCCAGGACCAGGTGGTGCCGG
ACAACACCCTGGCCTGGGTGTGGGTGCGCGGCCTGGACGAGCTGT
ACGCCGAGTGGTCGGAGGTCGTGTCCACGAACTTCCGGGACGCCT
CCGGGCCGGCCATGACCGAGATCGGCGAGCAGCCGTGGGGGGGGA
GTTCGCCCTGCGCGACCCGGCCGGCAACTGCGTGCACTTCGTGGC
CGAGGAGCAGGACTGA
Construct set 3: Hlalba-B4GALT1-GFP-Hglut1-ER
Sequence 1 (SEQ ID NO: 16):
hLALBA(SEQ ID NO: 1)-GGATCCG(linker)-
IRES2 (SEQ ID NO: 6)-hB4GALT1 (SEQ ID NO: 2)-
CTCGAG(linker)-IRES2 (SEQ ID NO: 6)-
GS (SEQ ID NO: 7)
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGAGGATCCGCCCCTCTCCCTCCC
CCCCCCCTAACGTTACTGGCCGAAGCCGCTTGGAATAAGGCCGGT
GTGCGTTTGTCTATATGTTATTTTCCACCATATTGCCGTCTTTTG
GCAATGTGAGGGCCCGGAAACCTGGCCCTGTCTTCTTGACGAGCA
TTCCTAGGGGTCTTTCCCCTCTCGCCAAAGGAATGCAAGGTCTGT
TGAATGTCGTGAAGGAAGCAGTTCCTCTGGAAGCTTCTTGAAGAC
AAACAACGTCTGTAGCGACCCTTTGCAGGCAGCGGAACCCCCCAC
CTGGCGACAGGTGCCTCTGCGGCCAAAAGCCACGTGTATAAGATA
CACCTGCAAAGGCGGCACAACCCCAGTGCCACGTTGTGAGTTGGA
TAGTTGTGGAAAGAGTCAAATGGCTCTCCTCAAGCGTATTCAACA
AGGGGCTGAAGGATGCCCAGAAGGTACCCCATTGTATGGGATCTG
ATCTGGGGCCTCGGTACACATGCTTTACATGTGTTTAGTCGAGGT
TAAAAAAACGTCTAGGCCCCCCGAACCACGGGGACGTGGTTTTCC
TTTGAAAAACACGATGATAATATGGCCACAACCATGAGGCTTCGG
GAGCCGCTCCTGAGCGGCAGCGCCGCGATGCCAGGCGCGTCCCTA
CAGCGGGCCTGCCGCCTGCTCGTGGCCGTCTGCGCTCTGCACCTT
GGCGTCACCCTCGTTTACTACCTGGCTGGCCGCGACCTGAGCCGC
CTGCCCCAACTGGTCGGAGTCTCCACACCGCTGCAGGGCGGCTCG
AACAGTGCCGCCGCCATCGGGCAGTCCTCCGGGGAGCTCCGGACC
GGAGGGGCCCGGCCGCCGCCTCCTCTAGGCGCCTCCTCCCAGCCG
CGCCCGGGTGGCGACTCCAGCCCAGTCGTGGATTCTGGCCCTGGC
CCCGCTAGCAACTTGACCTCGGTCCCAGTGCCCCACACCACCGCA
CTGTCGCTGCCCGCCTGCCCTGAGGAGTCCCCGCTGCTTGTGGGC
CCCATGCTGATTGAGTTTAACATGCCTGTGGACCTGGAGCTCGTG
GCAAAGCAGAACCCAAATGTGAAGATGGGCGGCCGCTATGCCCCC
AGGGACTGCGTCTCTCCTCACAAGGTGGCCATCATCATTCCATTC
CGCAACCGGCAGGAGCACCTCAAGTACTGGCTATATTATTTGCAC
CCAGTCCTGCAGCGCCAGCAGCTGGACTATGGCATCTATGTTATC
AACCAGGCGGGAGACACTATATTCAATCGTGCTAAGCTCCTCAAT
GTTGGCTTTCAAGAAGCCTTGAAGGACTATGACTACACCTGCTTT
GTGTTTAGTGACGTGGACCTCATTCCAATGAATGACCATAATGCG
TACAGGTGTTTTTCACAGCCACGGCACATTTCCGTTGCAATGGAT
AAGTTTGGATTCAGCCTACCTTATGTTCAGTATTTTGGAGGTGTC
TCTGCTCTAAGTAAACAACAGTTTCTAACCATCAATGGATTTCCT
AATAATTATTGGGGCTGGGGAGGAGAAGATGATGACATTTTTAAC
AGATTAGTTTTTAGAGGCATGTCTATATCTCGCCCAAATGCTGTG
GTCGGGAGGTGTCGCATGATCCGCCACTCAAGAGACAAGAAAAAT
GAACCCAATCCTCAGAGGTTTGACCGAATTGCACACACAAAGGAG
ACAATGCTCTCTGATGGTTTGAACTCACTCACCTACCAGGTGCTG
GATGTACAGAGATACCCATTGTATACCCAAATCACAGTGGACATC
GGGACACCGAGCTAGCTCGAGCCCCTCTCCCTCCCCCCCCCCTAA
CGTTACTGGCCGAAGCCGCTTGGAATAAGGCCGGTGTGCGTTTGT
CTATATGTTATTTTCCACCATATTGCCGTCTTTTGGCAATGTGAG
GGCCCGGAAACCTGGCCCTGTCTTCTTGACGAGCATTCCTAGGGG
TCTTTCCCCTCTCGCCAAAGGAATGCAAGGTCTGTTGAATGTCGT
GAAGGAAGCAGTTCCTCTGGAAGCTTCTTGAAGACAAACAACGTC
TGTAGCGACCCTTTGCAGGCAGCGGAACCCCCCACCTGGCGACAG
GTGCCTCTGCGGCCAAAAGCCACGTGTATAAGATACACCTGCAAA
GGCGGCACAACCCCAGTGCCACGTTGTGAGTTGGATAGTTGTGGA
AAGAGTCAAATGGCTCTCCTCAAGCGTATTCAACAAGGGGCTGAA
GGATGCCCAGAAGGTACCCCATTGTATGGGATCTGATCTGGGGCC
TCGGTACACATGCTTTACATGTGTTTAGTCGAGGTTAAAAAAACG
TCTAGGCCCCCCGAACCACGGGGACGTGGTTTTCCTTTGAAAAAC
ACGATGATAATATGGCCACAACCATGGCCACCTCAGCAAGTTCCC
ACTTGAACAAAAACATCAAGCAAATGTACTTGTGCCTGCCCCAGG
GTGAGAAAGTCCAAGCCATGTATATCTGGGTTGATGGTACTGGAG
AAGGACTGCGCTGCAAAACCCGCACCCTGGACTGTGAGCCCAAGT
GTGTAGAAGAGTTACCTGAGTGGAATTTTGATGGCTCTAGTACCT
TTCAGTCTGAGGGCTCCAACAGTGACATGTATCTCAGCCCTGTTG
CCATGTTTCGGGACCCCTTCCGCAGAGATCCCAACAAGCTGGTGT
TCTGTGAAGTTTTCAAGTACAACCGGAAGCCTGCAGAGACCAATT
TAAGGCACTCGTGTAAACGGATAATGGACATGGTGAGCAACCAGC
ACCCCTGGTTTGGAATGGAACAGGAGTATACTCTGATGGGAACAG
ATGGGCACCCTTTTGGTTGGCCTTCCAATGGCTTTCCTGGGCCCC
AAGGTCCGTATTACTGTGGTGTGGGCGCAGACAAAGCCTATGGCA
GGGATATCGTGGAGGCTCACTACCGCGCCTGCTTGTATGCTGGGG
TCAAGATTACAGGAACAAATGCTGAGGTCATGCCTGCCCAGTGGG
AATTCCAAATAGGACCCTGTGAAGGAATCCGCATGGGAGATCATC
TCTGGGTGGCCCGTTTCATCTTGCATCGAGTATGTGAAGACTTTG
GGGTAATAGCAACCTTTGACCCCAAGCCCATTCCTGGGAACTGGA
ATGGTGCAGGCTGCCATACCAACTTTAGCACCAAGGCCATGCGGG
AGGAGAATGGTCTGAAGCACATCGAGGAGGCCATCGAGAAACTAA
GCAAGCGGCACCGGTACCACATTCGAGCCTACGATCCCAAGGGGG
GCCTGGACAATGCCCGTCGTCTGACTGGGTTCCACGAAACGTCCA
ACATCAACGACTTTTCTGCTGGTGTCGCCAATCGCAGTGCCAGCA
TCTGCATTCCCCGGACTGTCGGCCAGGAGAAGAAAGGTTACTTTG
AAGACCGCCGCCCCTCTGCCAATTGTGACCCCTTTGCAGTGACAG
AAGCCATCGTCCGCACATGCCTTCTCAATGAGACTGGCGACGAGC
CCTTCCAATACAAAAACTAA
Sequence 2 (SEQ ID NO: 17):
eGFP(SEQ ID NO: 5)-hGLUT1(SEQ ID NO: 3)
(ER(SEQ ID NO: 10))-TGAGGCGCGCC(linker)-
IRES2 (SEQ ID NO: 6)-BleoR (SEQ ID NO: 8)
ATGGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGGACGGCAGCGTGCAGCTCGCC
GACCACTACCAGCAGAACACCCCCATCGGCGACGGCCCCGTGCTG
CTGCCCGACAACCACTACCTGAGCACCCAGTCCGCCCTGAGCAAA
GACCCCAACGAGAAGCGCGATCACATGGTCCTGCTGGAGTTCGTG
ACCGCCGCCGGGATCACTCTCGGCATGGACGAGCTGTACAAGGAG
CCCAGCAGCAAGAAGCTGACGGGTCGCCTCATGCTGGCCGTGGGA
GGAGCAGTGCTTGGCTCCCTGCAGTTTGGCTACAACACTGGAGTC
ATCAATGCCCCCCAGAAGGTGATCGAGGAGTTCTACAACCAGACA
TGGGTCCACCGCTATGGGGAGAGCATCCTGCCCACCACGCTCACC
ACGCTCTGGTCCCTCTCAGTGGCCATCTTTTCTGTTGGGGGCATG
ATTGGCTCCTTCTCTGTGGGCCTTTTCGTTAACCGCTTTGGCCGG
CGGAATTCAATGCTGATGATGAACCTGCTGGCCTTCGTGTCCGCC
GTGCTCATGGGCTTCTCGAAACTGGGCAAGTCCTTTGAGATGCTG
ATCCTGGGCCGCTTCATCATCGGTGTGTACTGCGGCCTGACCACA
GGCTTCGTGCCCATGTATGTGGGTGAAGTGTCACCCACAGCCCTT
CGTGGGGCCCTGGGCACCCTGCACCAGCTGGGCATCGTCGTCGGC
ATCCTCATCGCCCAGGTGTTCGGCCTGGACTCCATCATGGGCAAC
AAGGACCTGTGGCCCCTGCTGCTGAGCATCATCTTCATCCCGGCC
CTGCTGCAGTGCATCGTGCTGCCCTTCTGCCCCGAGAGTCCCCGC
TTCCTGCTCATCAACCGCAACGAGGAGAACCGGGCCAAGAGTGTG
CTAAAGAAGCTGCGCGGGACAGCTGACGTGACCCATGACCTGCAG
GAGATGAAGGAAGAGAGTCGGCAGATGATGCGGGAGAAGAAGGTC
ACCATCCTGGAGCTGTTCCGCTCCCCCGCCTACCGCCAGCCCATC
CTCATCGCTGTGGTGCTGCAGCTGTCCCAGCAGCTGTCTGGCATC
AACGCTGTCTTCTATTACTCCACGAGCATCTTCGAGAAGGCGGGG
GTGCAGCAGCCTGTGTATGCCACCATTGGCTCCGGTATCGTCAAC
ACGGCCTTCACTGTCGTGTCGCTGTTTGTGGTGGAGCGAGCAGGC
CGGCGGACCCTGCACCTCATAGGCCTCGCTGGCATGGGGGGTTGT
GCCATACTCATGACCATCGCGCTAGCACTGCTGGAGCAGCTACCC
TGGATGTCCTATCTGAGCATCGTGGCCATCTTTGGCTTTGTGGCC
TTCTTTGAAGTGGGTCCTGGCCCCATCCCATGGTTCATCGTGGCT
GAACTCTTCAGCCAGGGTCCACGTCCAGCTGCCATTGCCGTTGCA
GGCTTCTCCAACTGGACCTCAAATTTCATTGTGGGCATGTGCTTC
CAGTATGTGGAGCAACTGTGTGGTCCCTACGTCTTCATCATCTTC
ACTGTGCTCCTGGTTCTGTTCTTCATCTTCACCTACTTCAAAGTT
CCTGAGACTAAAGGCCGGACCTTCGATGAGATCGCTTCCGGCTTC
CGGCAGGGGGGAGCCAGCCAAAGTGACAAGACACCCGAGGAGCTG
TTCCATCCCCTGGGGGCTGATTCCCAAGTGCTGCTGACCAAGGTG
AAGGGCTCCTGAGGCGCGCCCCCCTCTCCCTCCCCCCCCCCTAAC
GTTACTGGCCGAAGCCGCTTGGAATAAGGCCGGTGTGCGTTTGTC
TATATGTTATTTTCCACCATATTGCCGTCTTTTGGCAATGTGAGG
GCCCGGAAACCTGGCCCTGTCTTCTTGACGAGCATTCCTAGGGGT
CTTTCCCCTCTCGCCAAAGGAATGCAAGGTCTGTTGAATGTCGTG
AAGGAAGCAGTTCCTCTGGAAGCTTCTTGAAGACAAACAACGTCT
GTAGCGACCCTTTGCAGGCAGCGGAACCCCCCACCTGGCGACAGG
TGCCTCTGCGGCCAAAAGCCACGTGTATAAGATACACCTGCAAAG
GCGGCACAACCCCAGTGCCACGTTGTGAGTTGGATAGTTGTGGAA
AGAGTCAAATGGCTCTCCTCAAGCGTATTCAACAAGGGGCTGAAG
GATGCCCAGAAGGTACCCCATTGTATGGGATCTGATCTGGGGCCT
CGGTACACATGCTTTACATGTGTTTAGTCGAGGTTAAAAAAACGT
CTAGGCCCCCCGAACCACGGGGACGTGGTTTTCCTTTGAAAAACA
CGATGATAATATGGCCACAACCATGGCCAAGTTGACCAGTGCCGT
TCCGGTGCTCACCGCGCGCGACGTCGCCGGAGCGGTCGAGTTCTG
GACCGACCGGCTCGGGTTCTCCCGGGACTTCGTGGAGGACGACTT
CGCCGGTGTGGTCCGGGACGACGTGACCCTGTTCATCAGCGCGGT
CCAGGACCAGGTGGTGCCGGACAACACCCTGGCCTGGGTGTGGGT
GCGCGGCCTGGACGAGCTGTACGCCGAGTGGTCGGAGGTCGTGTC
CACGAACTTCCGGGACGCCTCCGGGCCGGCCATGACCGAGATCGG
CGAGCAGCCGTGGGGGGGGGAGTTCGCCCTGCGCGACCCGGCCGG
CAACTGCGTGCACTTCGTGGCCGAGGAGCAGGACTGA
Construct set 4: hLALBA-B4GALT1-GFP-hGLUT1
(NT/LGNT)
Sequence 1 (SEQ ID NO: 18):
hLALBA(SEQ ID NO: 1)-GGATCCG(linker)-
IRES2(SEQ ID NO: 6)-B4GALT1(SEQ ID NO: 2)-
CTCGAG(linker)-IRES2 (SEQ ID NO: 6)-
GS (SEQ ID NO: 7)
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGAGGATCCGCCCCTCTCCCTCCC
CCCCCCCTAACGTTACTGGCCGAAGCCGCTTGGAATAAGGCCGGT
GTGCGTTTGTCTATATGTTATTTTCCACCATATTGCCGTCTTTTG
GCAATGTGAGGGCCCGGAAACCTGGCCCTGTCTTCTTGACGAGCA
TTCCTAGGGGTCTTTCCCCTCTCGCCAAAGGAATGCAAGGTCTGT
TGAATGTCGTGAAGGAAGCAGTTCCTCTGGAAGCTTCTTGAAGAC
AAACAACGTCTGTAGCGACCCTTTGCAGGCAGCGGAACCCCCCAC
CTGGCGACAGGTGCCTCTGCGGCCAAAAGCCACGTGTATAAGATA
CACCTGCAAAGGCGGCACAACCCCAGTGCCACGTTGTGAGTTGGA
TAGTTGTGGAAAGAGTCAAATGGCTCTCCTCAAGCGTATTCAACA
AGGGGCTGAAGGATGCCCAGAAGGTACCCCATTGTATGGGATCTG
ATCTGGGGCCTCGGTACACATGCTTTACATGTGTTTAGTCGAGGT
TAAAAAAACGTCTAGGCCCCCCGAACCACGGGGACGTGGTTTTCC
TTTGAAAAACACGATGATAATATGGCCACAACCATGAGGCTTCGG
GAGCCGCTCCTGAGCGGCAGCGCCGCGATGCCAGGCGCGTCCCTA
CAGCGGGCCTGCCGCCTGCTCGTGGCCGTCTGCGCTCTGCACCTT
GGCGTCACCCTCGTTTACTACCTGGCTGGCCGCGACCTGAGCCGC
CTGCCCCAACTGGTCGGAGTCTCCACACCGCTGCAGGGCGGCTCG
AACAGTGCCGCCGCCATCGGGCAGTCCTCCGGGGAGCTCCGGACC
GGAGGGGCCCGGCCGCCGCCTCCTCTAGGCGCCTCCTCCCAGCCG
CGCCCGGGTGGCGACTCCAGCCCAGTCGTGGATTCTGGCCCTGGC
CCCGCTAGCAACTTGACCTCGGTCCCAGTGCCCCACACCACCGCA
CTGTCGCTGCCCGCCTGCCCTGAGGAGTCCCCGCTGCTTGTGGGC
CCCATGCTGATTGAGTTTAACATGCCTGTGGACCTGGAGCTCGTG
GCAAAGCAGAACCCAAATGTGAAGATGGGCGGCCGCTATGCCCCC
AGGGACTGCGTCTCTCCTCACAAGGTGGCCATCATCATTCCATTC
CGCAACCGGCAGGAGCACCTCAAGTACTGGCTATATTATTTGCAC
CCAGTCCTGCAGCGCCAGCAGCTGGACTATGGCATCTATGTTATC
AACCAGGCGGGAGACACTATATTCAATCGTGCTAAGCTCCTCAAT
GTTGGCTTTCAAGAAGCCTTGAAGGACTATGACTACACCTGCTTT
GTGTTTAGTGACGTGGACCTCATTCCAATGAATGACCATAATGCG
TACAGGTGTTTTTCACAGCCACGGCACATTTCCGTTGCAATGGAT
AAGTTTGGATTCAGCCTACCTTATGTTCAGTATTTTGGAGGTGTC
TCTGCTCTAAGTAAACAACAGTTTCTAACCATCAATGGATTTCCT
AATAATTATTGGGGCTGGGGAGGAGAAGATGATGACATTTTTAAC
AGATTAGTTTTTAGAGGCATGTCTATATCTCGCCCAAATGCTGTG
GTCGGGAGGTGTCGCATGATCCGCCACTCAAGAGACAAGAAAAAT
GAACCCAATCCTCAGAGGTTTGACCGAATTGCACACACAAAGGAG
ACAATGCTCTCTGATGGTTTGAACTCACTCACCTACCAGGTGCTG
GATGTACAGAGATACCCATTGTATACCCAAATCACAGTGGACATC
GGGACACCGAGCTAGCTCGAGCCCCTCTCCCTCCCCCCCCCCTAA
CGTTACTGGCCGAAGCCGCTTGGAATAAGGCCGGTGTGCGTTTGT
CTATATGTTATTTTCCACCATATTGCCGTCTTTTGGCAATGTGAG
GGCCCGGAAACCTGGCCCTGTCTTCTTGACGAGCATTCCTAGGGG
TCTTTCCCCTCTCGCCAAAGGAATGCAAGGTCTGTTGAATGTCGT
GAAGGAAGCAGTTCCTCTGGAAGCTTCTTGAAGACAAACAACGTC
TGTAGCGACCCTTTGCAGGCAGCGGAACCCCCCACCTGGCGACAG
GTGCCTCTGCGGCCAAAAGCCACGTGTATAAGATACACCTGCAAA
GGCGGCACAACCCCAGTGCCACGTTGTGAGTTGGATAGTTGTGGA
AAGAGTCAAATGGCTCTCCTCAAGCGTATTCAACAAGGGGCTGAA
GGATGCCCAGAAGGTACCCCATTGTATGGGATCTGATCTGGGGCC
TCGGTACACATGCTTTACATGTGTTTAGTCGAGGTTAAAAAAACG
TCTAGGCCCCCCGAACCACGGGGACGTGGTTTTCCTTTGAAAAAC
ACGATGATAATATGGCCACAACCATGGCCACCTCAGCAAGTTCCC
ACTTGAACAAAAACATCAAGCAAATGTACTTGTGCCTGCCCCAGG
GTGAGAAAGTCCAAGCCATGTATATCTGGGTTGATGGTACTGGAG
AAGGACTGCGCTGCAAAACCCGCACCCTGGACTGTGAGCCCAAGT
GTGTAGAAGAGTTACCTGAGTGGAATTTTGATGGCTCTAGTACCT
TTCAGTCTGAGGGCTCCAACAGTGACATGTATCTCAGCCCTGTTG
CCATGTTTCGGGACCCCTTCCGCAGAGATCCCAACAAGCTGGTGT
TCTGTGAAGTTTTCAAGTACAACCGGAAGCCTGCAGAGACCAATT
TAAGGCACTCGTGTAAACGGATAATGGACATGGTGAGCAACCAGC
ACCCCTGGTTTGGAATGGAACAGGAGTATACTCTGATGGGAACAG
ATGGGCACCCTTTTGGTTGGCCTTCCAATGGCTTTCCTGGGCCCC
AAGGTCCGTATTACTGTGGTGTGGGCGCAGACAAAGCCTATGGCA
GGGATATCGTGGAGGCTCACTACCGCGCCTGCTTGTATGCTGGGG
TCAAGATTACAGGAACAAATGCTGAGGTCATGCCTGCCCAGTGGG
AATTCCAAATAGGACCCTGTGAAGGAATCCGCATGGGAGATCATC
TCTGGGTGGCCCGTTTCATCTTGCATCGAGTATGTGAAGACTTTG
GGGTAATAGCAACCTTTGACCCCAAGCCCATTCCTGGGAACTGGA
ATGGTGCAGGCTGCCATACCAACTTTAGCACCAAGGCCATGCGGG
AGGAGAATGGTCTGAAGCACATCGAGGAGGCCATCGAGAAACTAA
GCAAGCGGCACCGGTACCACATTCGAGCCTACGATCCCAAGGGGG
GCCTGGACAATGCCCGTCGTCTGACTGGGTTCCACGAAACGTCCA
ACATCAACGACTTTTCTGCTGGTGTCGCCAATCGCAGTGCCAGCA
TCTGCATTCCCCGGACTGTCGGCCAGGAGAAGAAAGGTTACTTTG
AAGACCGCCGCCCCTCTGCCAATTGTGACCCCTTTGCAGTGACAG
AAGCCATCGTCCGCACATGCCTTCTCAATGAGACTGGCGACGAGC
CCTTCCAATACAAAAACTAA
Sequence 2 (SEQ ID NO: 19):
eGFP(SEQ ID NO: 5)-hGLUT1(SEQ ID NO: 3)-
IRES2(SEQ ID NO: 6)-BleoR(SEQ ID NO: 8)
ATGGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGGACGGCAGCGTGCAGCTCGCC
GACCACTACCAGCAGAACACCCCCATCGGCGACGGCCCCGTGCTG
CTGCCCGACAACCACTACCTGAGCACCCAGTCCGCCCTGAGCAAA
GACCCCAACGAGAAGCGCGATCACATGGTCCTGCTGGAGTTCGTG
ACCGCCGCCGGGATCACTCTCGGCATGGACGAGCTGTACAAGGAG
CCCAGCAGCAAGAAGCTGACGGGTCGCCTCATGCTGGCCGTGGGA
GGAGCAGTGCTTGGCTCCCTGCAGTTTGGCTACAACACTGGAGTC
ATCAATGCCCCCCAGAAGGTGATCGAGGAGTTCTACAACCAGACA
TGGGTCCACCGCTATGGGGAGAGCATCCTGCCCACCACGCTCACC
ACGCTCTGGTCCCTCTCAGTGGCCATCTTTTCTGTTGGGGGCATG
ATTGGCTCCTTCTCTGTGGGCCTTTTCGTTAACCGCTTTGGCCGG
CGGAATTCAATGCTGATGATGAACCTGCTGGCCTTCGTGTCCGCC
GTGCTCATGGGCTTCTCGAAACTGGGCAAGTCCTTTGAGATGCTG
ATCCTGGGCCGCTTCATCATCGGTGTGTACTGCGGCCTGACCACA
GGCTTCGTGCCCATGTATGTGGGTGAAGTGTCACCCACAGCCCTT
CGTGGGGCCCTGGGCACCCTGCACCAGCTGGGCATCGTCGTCGGC
ATCCTCATCGCCCAGGTGTTCGGCCTGGACTCCATOATGGGCAAC
AAGGACCTGTGGCCCCTGCTGCTGAGCATCATCTTCATCCCGGCC
CTGCTGCAGTGCATCGTGCTGCCCTTCTGCCCCGAGAGTCCCCGC
TTCCTGCTCATCAACCGCAACGAGGAGAACCGGGCCAAGAGTGTG
CTAAAGAAGCTGCGCGGGACAGCTGACGTGACCCATGACCTGCAG
GAGATGAAGGAAGAGAGTCGGCAGATGATGCGGGAGAAGAAGGTC
ACCATCCTGGAGCTGTTCCGCTCCCCCGCCTACCGCCAGCCCATC
CTCATCGCTGTGGTGCTGCAGCTGTCCCAGCAGCTGTCTGGCATC
AACGCTGTCTTCTATTACTCCACGAGCATCTTCGAGAAGGCGGGG
GTGCAGCAGCCTGTGTATGCCACCATTGGCTCCGGTATCGTCAAC
ACGGCCTTCACTGTCGTGTCGCTGTTTGTGGTGGAGCGAGCAGGC
CGGCGGACCCTGCACCTCATAGGCCTCGCTGGCATGGCGGGTTGT
GCCATACTCATGACCATCGCGCTAGCACTGCTGGAGCAGCTACCC
TGGATGTCCTATCTGAGCATCGTGGCCATCTTTGGCTTTGTGGCC
TTCTTTGAAGTGGGTCCTGGCCCCATCCCATGGTTCATCGTGGCT
GAACTCTTCAGCCAGGGTCCACGTCCAGCTGCCATTGCCGTTGCA
GGCTTCTCCAACTGGACCTCAAATTTCATTGTGGGCATGTGCTTC
CAGTATGTGGAGCAACTGTGTGGTCCCTACGTCTTCATCATCTTC
ACTGTGCTCCTGGTTCTGTTCTTCATCTTCACCTACTTCAAAGTT
CCTGAGACTAAAGGCCGGACCTTCGATGAGATCGCTTCCGGCTTC
CGGCAGGGGGGAGCCAGCCAAAGTGACAAGACACCCGAGGAGCTG
TTCCATCCCCTGGGGGCTGATTCCCAAGTGTGAGGCGCGCCCCCC
TCTCCCTCCCCCCCCCCTAACGTTACTGGCCGAAGCCGCTTGGAA
TAAGGCCGGTGTGCGTTTGTCTATATGTTATTTTCCACCATATTG
CCGTCTTTTGGCAATGTGAGGGCCCGGAAACCTGGCCCTGTCTTC
TTGACGAGCATTCCTAGGGGTCTTTCCCCTCTCGCCAAAGGAATG
CAAGGTCTGTTGAATGTCGTGAAGGAAGCAGTTCCTCTGGAAGCT
TCTTGAAGACAAACAACGTCTGTAGCGACCCTTTGCAGGCAGCGG
AACCCCCCACCTGGCGACAGGTGCCTCTGCGGCCAAAAGCCACGT
GTATAAGATACACCTGCAAAGGCGGCACAACCCCAGTGCCACGTT
GTGAGTTGGATAGTTGTGGAAAGAGTCAAATGGCTCTCCTCAAGC
GTATTCAACAAGGGGCTGAAGGATGCCCAGAAGGTACCCCATTGT
ATGGGATCTGATCTGGGGCCTCGGTACACATGCTTTACATGTGTT
TAGTCGAGGTTAAAAAAACGTCTAGGCCCCCCGAACCACGGGGAC
GTGGTTTTCCTTTGAAAAACACGATGATAATATGGCCACAACCAT
GGCCAAGTTGACCAGTGCCGTTCCGGTGCTCACCGCGCGCGACGT
CGCCGGAGCGGTCGAGTTCTGGACCGACCGGCTCGGGTTCTCCCG
GGACTTCGTGGAGGACGACTTCGCCGGTGTGGTCCGGGACGACGT
GACCCTGTTCATCAGCGCGGTCCAGGACCAGGTGGTGCCGGACAA
CACCCTGGCCTGGGTGTGGGTGCGCGGCCTGGACGAGCTGTACGC
CGAGTGGTCGGAGGTCGTGTCCACGAACTTCCGGGACGCCTCCGG
GCCGGCCATGACCGAGATCGGCGAGCAGCCGTGGGGGGGGGAGTT
CGCCCTGCGCGACCCGGCCGGCAACTGCGTGCACTTCGTGGCCGA
GGAGCAGGACTGA
Construct set 5: hLALBA
Sequence 1 (SEQ ID NO: 20):
hLALBA(SEQ ID NO: 1)-CTCGAG(linker)-
IRES2(SEQ ID NO: 6)-GS(SEQ ID NO: 7)
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGACTCGAGCCCCTCTCCCTCCCC
CCCCCCTAACGTTACTGGCCGAAGCCGCTTGGAATAAGGCCGGTG
TGCGTTTGTCTATATGTTATTTTCCACCATATTGCCGTCTTTTGG
CAATGTGAGGGCCCGGAAACCTGGCCCTGTCTTCTTGACGAGCAT
TCCTAGGGGTCTTTCCCCTCTCGCCAAAGGAATGCAAGGTCTGTT
GAATGTCGTGAAGGAAGCAGTTCCTCTGGAAGCTTCTTGAAGACA
AACAACGTCTGTAGCGACCCTTTGCAGGCAGCGGAACCCCCCACC
TGGCGACAGGTGCCTCTGCGGCCAAAAGCCACGTGTATAAGATAC
ACCTGCAAAGGCGGCACAACCCCAGTGCCACGTTGTGAGTTGGAT
AGTTGTGGAAAGAGTCAAATGGCTCTCCTCAAGCGTATTCAACAA
GGGGCTGAAGGATGCCCAGAAGGTACCCCATTGTATGGGATCTGA
TCTGGGGCCTCGGTACACATGCTTTACATGTGTTTAGTCGAGGTT
AAAAAAACGTCTAGGCCCCCCGAACCACGGGGACGTGGTTTTCCT
TTGAAAAACACGATGATAATATGGCCACAACCATGGCCACCTCAG
CAAGTTCCCACTTGAACAAAAACATCAAGCAAATGTACTTGTGCC
TGCCCCAGGGTGAGAAAGTCCAAGCCATGTATATCTGGGTTGATG
GTACTGGAGAAGGACTGCGCTGCAAAACCCGCACCCTGGACTGTG
AGCCCAAGTGTGTAGAAGAGTTACCTGAGTGGAATTTTGATGGCT
CTAGTACCTTTCAGTCTGAGGGCTCCAACAGTGACATGTATCTCA
GCCCTGTTGCCATGTTTCGGGACCCCTTCCGCAGAGATCCCAACA
AGCTGGTGTTCTGTGAAGTTTTCAAGTACAACCGGAAGCCTGCAG
AGACCAATTTAAGGCACTCGTGTAAACGGATAATGGACATGGTGA
GCAACCAGCACCCCTGGTTTGGAATGGAACAGGAGTATACTCTGA
TGGGAACAGATGGGCACCCTTTTGGTTGGCCTTCCAATGGCTTTC
CTGGGCCCCAAGGTCCGTATTACTGTGGTGTGGGCGCAGACAAAG
CCTATGGCAGGGATATCGTGGAGGCTCACTACCGCGCCTGCTTGT
ATGCTGGGGTCAAGATTACAGGAACAAATGCTGAGGTCATGCCTG
CCCAGTGGGAATTCCAAATAGGACCCTGTGAAGGAATCCGCATGG
GAGATCATCTCTGGGTGGCCCGTTTCATCTTGCATCGAGTATGTG
AAGACTTTGGGGTAATAGCAACCTTTGACCCCAAGCCCATTCCTG
GGAACTGGAATGGTGCAGGCTGCCATACCAACTTTAGCACCAAGG
CCATGCGGGAGGAGAATGGTCTGAAGCACATCGAGGAGGCCATCG
AGAAACTAAGCAAGCGGCACCGGTACCACATTCGAGCCTACGATC
CCAAGGGGGGCCTGGACAATGCCCGTCGTCTGACTGGGTTCCACG
AAACGTCCAACATCAACGACTTTTCTGCTGGTGTCGCCAATCGCA
GTGCCAGCATCTGCATTCCCCGGACTGTCGGCCAGGAGAAGAAAG
GTTACTTTGAAGACCGCCGCCCCTCTGCCAATTGTGACCCCTTTG
CAGTGACAGAAGCCATCGTCCGCACATGCCTTCTCAATGAGACTG
GCGACGAGCCCTTCCAATACAAAAACTAA
Sequence 2 (SEQ ID NO: 21):
IRES2(SEQ ID NO: 6)-BleoR (SEQ ID NO: 8)
CCCCTCTCCCTCCCCCCCCCCTAACGTTACTGGCCGAAGCCGCTT
GGAATAAGGCCGGTGTGCGTTTGTCTATATGTTATTTTCCACCAT
ATTGCCGTCTTTTGGCAATGTGAGGGCCCGGAAACCTGGCCCTGT
CTTCTTGACGAGCATTCCTAGGGGTCTTTCCCCTCTCGCCAAAGG
AATGCAAGGTCTGTTGAATGTCGTGAAGGAAGCAGTTCCTCTGGA
AGCTTCTTGAAGACAAACAACGTCTGTAGCGACCCTTTGCAGGCA
GCGGAACCCCCCACCTGGCGACAGGTGCCTCTGCGGCCAAAAGCC
ACGTGTATAAGATACACCTGCAAAGGCGGCACAACCCCAGTGCCA
CGTTGTGAGTTGGATAGTTGTGGAAAGAGTCAAATGGCTCTCCTC
AAGCGTATTCAACAAGGGGCTGAAGGATGCCCAGAAGGTACCCCA
TTGTATGGGATCTGATCTGGGGCCTCGGTACACATGCTTTACATG
TGTTTAGTCGAGGTTAAAAAAACGTCTAGGCCCCCCGAACCACGG
GGACGTGGTTTTCCTTTGAAAAACACGATGATAATATGGCCACAA
CCATGGCCAAGTTGACCAGTGCCGTTCCGGTGCTCACCGCGCGCG
ACGTCGCCGGAGCGGTCGAGTTCTGGACCGACCGGCTCGGGTTCT
CCCGGGACTTCGTGGAGGACGACTTCGCCGGTGTGGTCCGGGACG
ACGTGACCCTGTTCATCAGCGCGGTCCAGGACCAGGTGGTGCCGG
ACAACACCCTGGCCTGGGTGTGGGTGCGCGGCCTGGACGAGCTGT
ACGCCGAGTGGTCGGAGGTCGTGTCCACGAACTTCCGGGACGCCT
CCGGGCCGGCCATGACCGAGATCGGCGAGCAGCCGTGGGGGGGGA
GTTCGCCCTGCGCGACCCGGCCGGCAACTGCGTGCACTTCGTGGC
CGAGGAGCAGGACTGA
Construct set 6: hB4GALT1
Sequence 1(SEQ ID NO: 22):
hB4GALT1(SEQ ID NO: 2)-CTCGAG(linker)-
IRES2(SEQ ID NO: 6)-GS(SEQ ID NO: 7)
ATGAGGCTTCGGGAGCCGCTCCTGAGCGGCAGCGCCGCGATGCCA
GGCGCGTCCCTACAGCGGGCCTGCCGCCTGCTCGTGGCCGTCTGC
GCTCTGCACCTTGGCGTCACCCTCGTTTACTACCTGGCTGGCCGC
GACCTGAGCCGCCTGCCCCAACTGGTCGGAGTCTCCACACCGCTG
CAGGGCGGCTCGAACAGTGCCGCCGCCATCGGGCAGTCCTCCGGG
GAGCTCCGGACCGGAGGGGCCCGGCCGCCGCCTCCTCTAGGCGCC
TCCTCCCAGCCGCGCCCGGGTGGCGACTCCAGCCCAGTCGTGGAT
TCTGGCCCTGGCCCCGCTAGCAACTTGACCTCGGTCCCAGTGCCC
CACACCACCGCACTGTCGCTGCCCGCCTGCCCTGAGGAGTCCCCG
CTGCTTGTGGGCCCCATGCTGATTGAGTTTAACATGCCTGTGGAC
CTGGAGCTCGTGGCAAAGCAGAACCCAAATGTGAAGATGGGCGGC
CGCTATGCCCCCAGGGACTGCGTCTCTCCTCACAAGGTGGCCATC
ATCATTCCATTCCGCAACCGGCAGGAGCACCTCAAGTACTGGCTA
TATTATTTGCACCCAGTCCTGCAGCGCCAGCAGCTGGACTATGGC
ATCTATGTTATCAACCAGGCGGGAGACACTATATTCAATCGTGCT
AAGCTCCTCAATGTTGGCTTTCAAGAAGCCTTGAAGGACTATGAC
TACACCTGCTTTGTGTTTAGTGACGTGGACCTCATTCCAATGAAT
GACCATAATGCGTACAGGTGTTTTTCACAGCCACGGCACATTTCC
GTTGCAATGGATAAGTTTGGATTCAGCCTACCTTATGTTCAGTAT
TTTGGAGGTGTCTCTGCTCTAAGTAAACAACAGTTTCTAACCATC
AATGGATTTCCTAATAATTATTGGGGCTGGGGAGGAGAAGATGAT
GACATTTTTAACAGATTAGTTTTTAGAGGCATGTCTATATCTCGC
CCAAATGCTGTGGTCGGGAGGTGTCGCATGATCCGCCACTCAAGA
GACAAGAAAAATGAACCCAATCCTCAGAGGTTTGACCGAATTGCA
CACACAAAGGAGACAATGCTCTCTGATGGTTTGAACTCACTCACC
TACCAGGTGCTGGATGTACAGAGATACCCATTGTATACCCAAATC
ACAGTGGACATCGGGACACCGAGCTAGctcgagCCCCTCTCCCTC
CCCCCCCCCTAACGTTACTGGCCGAAGCCGCTTGGAATAAGGCCG
GTGTGCGTTTGTCTATATGTTATTTTCCACCATATTGCCGTCTTT
TGGCAATGTGAGGGCCCGGAAACCTGGCCCTGTCTTCTTGACGAG
CATTCCTAGGGGTCTTTCCCCTCTCGCCAAAGGAATGCAAGGTCT
GTTGAATGTCGTGAAGGAAGCAGTTCCTCTGGAAGCTTCTTGAAG
ACAAACAACGTCTGTAGCGACCCTTTGCAGGCAGCGGAACCCCCC
ACCTGGCGACAGGTGCCTCTGCGGCCAAAAGCCACGTGTATAAGA
TACACCTGCAAAGGCGGCACAACCCCAGTGCCACGTTGTGAGTTG
GATAGTTGTGGAAAGAGTCAAATGGCTCTCCTCAAGCGTATTCAA
CAAGGGGCTGAAGGATGCCCAGAAGGTACCCCATTGTATGGGATC
TGATCTGGGGCCTCGGTACACATGCTTTACATGTGTTTAGTCGAG
GTTAAAAAAACGTCTAGGCCCCCCGAACCACGGGGACGTGGTTTT
CCTTTGAAAAACACGATGATAATATGGCCACAACCATGGCCACCT
CAGCAAGTTCCCACTTGAACAAAAACATCAAGCAAATGTACTTGT
GCCTGCCCCAGGGTGAGAAAGTCCAAGCCATGTATATCTGGGTTG
ATGGTACTGGAGAAGGACTGCGCTGCAAAACCCGCACCCTGGACT
GTGAGCCCAAGTGTGTAGAAGAGTTACCTGAGTGGAATTTTGATG
GCTCTAGTACCTTTCAGTCTGAGGGCTCCAACAGTGACATGTATC
TCAGCCCTGTTGCCATGTTTCGGGACCCCTTCCGCAGAGATCCCA
ACAAGCTGGTGTTCTGTGAAGTTTTCAAGTACAACCGGAAGCCTG
CAGAGACCAATTTAAGGCACTCGTGTAAACGGATAATGGACATGG
TGAGCAACCAGCACCCCTGGTTTGGAATGGAACAGGAGTATACTC
TGATGGGAACAGATGGGCACCCTTTTGGTTGGCCTTCCAATGGCT
TTCCTGGGCCCCAAGGTCCGTATTACTGTGGTGTGGGCGCAGACA
AAGCCTATGGCAGGGATATCGTGGAGGCTCACTACCGCGCCTGCT
TGTATGCTGGGGTCAAGATTACAGGAACAAATGCTGAGGTCATGC
CTGCCCAGTGGGAATTCCAAATAGGACCCTGTGAAGGAATCCGCA
TGGGAGATCATCTCTGGGTGGCCCGTTTCATCTTGCATCGAGTAT
GTGAAGACTTTGGGGTAATAGCAACCTTTGACCCCAAGCCCATTC
CTGGGAACTGGAATGGTGCAGGCTGCCATACCAACTTTAGCACCA
AGGCCATGCGGGAGGAGAATGGTCTGAAGCACATCGAGGAGGCCA
TCGAGAAACTAAGCAAGCGGCACCGGTACCACATTCGAGCCTACG
ATCCCAAGGGGGGCCTGGACAATGCCCGTCGTCTGACTGGGTTCC
ACGAAACGTCCAACATCAACGACTTTTCTGCTGGTGTCGCCAATC
GCAGTGCCAGCATCTGCATTCCCCGGACTGTCGGCCAGGAGAAGA
AAGGTTACTTTGAAGACCGCCGCCCCTCTGCCAATTGTGACCCCT
TTGCAGTGACAGAAGCCATCGTCCGCACATGCCTTCTCAATGAGA
CTGGCGACGAGCCCTTCCAATACAAAAACTAA
Sequence 2 (SEQ ID NO: 23):
IRES2 (SEQ ID NO: 6)-BleoR (SEQ ID NO: 8)
CCCCTCTCCCTCCCCCCCCCCTAACGTTACTGGCCGAAGCCGCTT
GGAATAAGGCCGGTGTGCGTTTGTCTATATGTTATTTTCCACCAT
ATTGCCGTCTTTTGGCAATGTGAGGGCCCGGAAACCTGGCCCTGT
CTTCTTGACGAGCATTCCTAGGGGTCTTTCCCCTCTCGCCAAAGG
AATGCAAGGTCTGTTGAATGTCGTGAAGGAAGCAGTTCCTCTGGA
AGCTTCTTGAAGACAAACAACGTCTGTAGCGACCCTTTGCAGGCA
GCGGAACCCCCCACCTGGCGACAGGTGCCTCTGCGGCCAAAAGCC
ACGTGTATAAGATACACCTGCAAAGGCGGCACAACCCCAGTGCCA
CGTTGTGAGTTGGATAGTTGTGGAAAGAGTCAAATGGCTCTCCTC
AAGCGTATTCAACAAGGGGCTGAAGGATGCCCAGAAGGTACCCCA
TTGTATGGGATCTGATCTGGGGCCTCGGTACACATGCTTTACATG
TGTTTAGTCGAGGTTAAAAAAACGTCTAGGCCCCCCGAACCACGG
GGACGTGGTTTTCCTTTGAAAAACACGATGATAATATGGCCACAA
CCATGGCCAAGTTGACCAGTGCCGTTCCGGTGCTCACCGCGCGCG
ACGTCGCCGGAGCGGTCGAGTTCTGGACCGACCGGCTCGGGTTCT
CCCGGGACTTCGTGGAGGACGACTTCGCCGGTGTGGTCCGGGACG
ACGTGACCCTGTTCATCAGCGCGGTCCAGGACCAGGTGGTGCCGG
ACAACACCCTGGCCTGGGTGTGGGTGCGCGGCCTGGACGAGCTGT
ACGCCGAGTGGTCGGAGGTCGTGTCCACGAACTTCCGGGACGCCT
CCGGGCCGGCCATGACCGAGATCGGCGAGCAGCCGTGGGGGGGGA
GTTCGCCCTGCGCGACCCGGCCGGCAACTGCGTGCACTTCGTGGC
CGAGGAGCAGGACTGA
Construct set 7: hLALBA-B4GALT1-GFP-
hGLUT1Δ(ST5)
Sequence 1 (SEQ ID NO: 24):
hLALBA(SEQ ID NO: 1)-GGATCCG(linker)-
IRES2(SEQ ID NO: 6)-hB4GALT1(SEQ ID
NO: 2)-CTCGAG(linker)-IRES2 (SEQ ID NO: 6)-
GS (SEQ ID NO: 7)
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGAGGATCCGCCCCTCTCCCTCCC
CCCCCCCTAACGTTACTGGCCGAAGCCGCTTGGAATAAGGCCGGT
GTGCGTTTGTCTATATGTTATTTTCCACCATATTGCCGTCTTTTG
GCAATGTGAGGGCCCGGAAACCTGGCCCTGTCTTCTTGACGAGCA
TTCCTAGGGGTCTTTCCCCTCTCGCCAAAGGAATGCAAGGTCTGT
TGAATGTCGTGAAGGAAGCAGTTCCTCTGGAAGCTTCTTGAAGAC
AAACAACGTCTGTAGCGACCCTTTGCAGGCAGCGGAACCCCCCAC
CTGGCGACAGGTGCCTCTGCGGCCAAAAGCCACGTGTATAAGATA
CACCTGCAAAGGCGGCACAACCCCAGTGCCACGTTGTGAGTTGGA
TAGTTGTGGAAAGAGTCAAATGGCTCTCCTCAAGCGTATTCAACA
AGGGGCTGAAGGATGCCCAGAAGGTACCCCATTGTATGGGATCTG
ATCTGGGGCCTCGGTACACATGCTTTACATGTGTTTAGTCGAGGT
TAAAAAAACGTCTAGGCCCCCCGAACCACGGGGACGTGGTTTTCC
TTTGAAAAACACGATGATAATATGGCCACAACCATGAGGCTTCGG
GAGCCGCTCCTGAGCGGCAGCGCCGCGATGCCAGGCGCGTCCCTA
CAGCGGGCCTGCCGCCTGCTCGTGGCCGTCTGCGCTCTGCACCTT
GGCGTCACCCTCGTTTACTACCTGGCTGGCCGCGACCTGAGCCGC
CTGCCCCAACTGGTCGGAGTCTCCACACCGCTGCAGGGCGGCTCG
AACAGTGCCGCCGCCATCGGGCAGTCCTCCGGGGAGCTCCGGACC
GGAGGGGCCCGGCCGCCGCCTCCTCTAGGCGCCTCCTCCCAGCCG
CGCCCGGGTGGCGACTCCAGCCCAGTCGTGGATTCTGGCCCTGGC
CCCGCTAGCAACTTGACCTCGGTCCCAGTGCCCCACACCACCGCA
CTGTCGCTGCCCGCCTGCCCTGAGGAGTCCCCGCTGCTTGTGGGC
CCCATGCTGATTGAGTTTAACATGCCTGTGGACCTGGAGCTCGTG
GCAAAGCAGAACCCAAATGTGAAGATGGGCGGCCGCTATGCCCCC
AGGGACTGCGTCTCTCCTCACAAGGTGGCCATCATCATTCCATTC
CGCAACCGGCAGGAGCACCTCAAGTACTGGCTATATTATTTGCAC
CCAGTCCTGCAGCGCCAGCAGCTGGACTATGGCATCTATGTTATC
AACCAGGCGGGAGACACTATATTCAATCGTGCTAAGCTCCTCAAT
GTTGGCTTTCAAGAAGCCTTGAAGGACTATGACTACACCTGCTTT
GTGTTTAGTGACGTGGACCTCATTCCAATGAATGACCATAATGCG
TACAGGTGTTTTTCACAGCCACGGCACATTTCCGTTGCAATGGAT
AAGTTTGGATTCAGCCTACCTTATGTTCAGTATTTTGGAGGTGTC
TCTGCTCTAAGTAAACAACAGTTTCTAACCATCAATGGATTTCCT
AATAATTATTGGGGCTGGGGAGGAGAAGATGATGACATTTTTAAC
AGATTAGTTTTTAGAGGCATGTCTATATCTCGCCCAAATGCTGTG
GTCGGGAGGTGTCGCATGATCCGCCACTCAAGAGACAAGAAAAAT
GAACCCAATCCTCAGAGGTTTGACCGAATTGCACACACAAAGGAG
ACAATGCTCTCTGATGGTTTGAACTCACTCACCTACCAGGTGCTG
GATGTACAGAGATACCCATTGTATACCCAAATCACAGTGGACATC
GGGACACCGAGCTAGCTCGAGCCCCTCTCCCTCCCCCCCCCCTAA
CGTTACTGGCCGAAGCCGCTTGGAATAAGGCCGGTGTGCGTTTGT
CTATATGTTATTTTCCACCATATTGCCGTCTTTTGGCAATGTGAG
GGCCCGGAAACCTGGCCCTGTCTTCTTGACGAGCATTCCTAGGGG
TCTTTCCCCTCTCGCCAAAGGAATGCAAGGTCTGTTGAATGTCGT
GAAGGAAGCAGTTCCTCTGGAAGCTTCTTGAAGACAAACAACGTC
TGTAGCGACCCTTTGCAGGCAGCGGAACCCCCCACCTGGCGACAG
GTGCCTCTGCGGCCAAAAGCCACGTGTATAAGATACACCTGCAAA
GGCGGCACAACCCCAGTGCCACGTTGTGAGTTGGATAGTTGTGGA
AAGAGTCAAATGGCTCTCCTCAAGCGTATTCAACAAGGGGCTGAA
GGATGCCCAGAAGGTACCCCATTGTATGGGATCTGATCTGGGGCC
TCGGTACACATGCTTTACATGTGTTTAGTCGAGGTTAAAAAAACG
TCTAGGCCCCCCGAACCACGGGGACGTGGTTTTCCTTTGAAAAAC
ACGATGATAATATGGCCACAACCATGGCCACCTCAGCAAGTTCCC
ACTTGAACAAAAACATCAAGCAAATGTACTTGTGCCTGCCCCAGG
GTGAGAAAGTCCAAGCCATGTATATCTGGGTTGATGGTACTGGAG
AAGGACTGCGCTGCAAAACCCGCACCCTGGACTGTGAGCCCAAGT
GTGTAGAAGAGTTACCTGAGTGGAATTTTGATGGCTCTAGTACCT
TTCAGTCTGAGGGCTCCAACAGTGACATGTATCTCAGCCCTGTTG
CCATGTTTCGGGACCCCTTCCGCAGAGATCCCAACAAGCTGGTGT
TCTGTGAAGTTTTCAAGTACAACCGGAAGCCTGCAGAGACCAATT
TAAGGCACTCGTGTAAACGGATAATGGACATGGTGAGCAACCAGC
ACCCCTGGTTTGGAATGGAACAGGAGTATACTCTGATGGGAACAG
ATGGGCACCCTTTTGGTTGGCCTTCCAATGGCTTTCCTGGGCCCC
AAGGTCCGTATTACTGTGGTGTGGGCGCAGACAAAGCCTATGGCA
GGGATATCGTGGAGGCTCACTACCGCGCCTGCTTGTATGCTGGGG
TCAAGATTACAGGAACAAATGCTGAGGTCATGCCTGCCCAGTGGG
AATTCCAAATAGGACCCTGTGAAGGAATCCGCATGGGAGATCATC
TCTGGGTGGCCCGTTTCATCTTGCATCGAGTATGTGAAGACTTTG
GGGTAATAGCAACCTTTGACCCCAAGCCCATTCCTGGGAACTGGA
ATGGTGCAGGCTGCCATACCAACTTTAGCACCAAGGCCATGCGGG
AGGAGAATGGTCTGAAGCACATCGAGGAGGCCATCGAGAAACTAA
GCAAGCGGCACCGGTACCACATTCGAGCCTACGATCCCAAGGGGG
GCCTGGACAATGCCCGTCGTCTGACTGGGTTCCACGAAACGTCCA
ACATCAACGACTTTTCTGCTGGTGTCGCCAATCGCAGTGCCAGCA
TCTGCATTCCCCGGACTGTCGGCCAGGAGAAGAAAGGTTACTTTG
AAGACCGCCGCCCCTCTGCCAATTGTGACCCCTTTGCAGTGACAG
AAGCCATCGTCCGCACATGCCTTCTCAATGAGACTGGCGACGAGC
CCTTCCAATACAAAAACTAA
Sequence 2 (SEQ ID NO: 25):
eGFP(SEQ ID NO: 5)-hGLUT1Δ(SEQ ID NO: 4)-
GGCGCGCC(linker)-IRES2(SEQ ID NO: 6)-
BleoR (SEQ ID NO: 8)
ATGGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGGACGGCAGCGTGCAGCTCGCC
GACCACTACCAGCAGAACACCCCCATCGGCGACGGCCCCGTGCTG
CTGCCCGACAACCACTACCTGAGCACCCAGTCCGCCCTGAGCAAA
GACCCCAACGAGAAGCGCGATCACATGGTCCTGCTGGAGTTCGTG
ACCGCCGCCGGGATCACTCTCGGCATGGACGAGCTGTACAAGGAG
CCCAGCAGCAAGAAGCTGACGGGTCGCCTCATGCTGGCCGTGGGA
GGAGCAGTGCTTGGCTCCCTGCAGTTTGGCTACAACACTGGAGTC
ATCAATGCCCCCCAGAAGGTGATCGAGGAGTTCTACAACCAGACA
TGGGTCCACCGCTATGGGGAGAGCATCCTGCCCACCACGCTCACC
ACGCTCTGGTCCCTCTCAGTGGCCATCTTTTCTGTTGGGGGCATG
ATTGGCTCCTTCTCTGTGGGCCTTTTCGTTAACCGCTTTGGCCGG
CGGAATTCAATGCTGATGATGAACCTGCTGGCCTTCGTGTCCGCC
GTGCTCATGGGCTTCTCGAAACTGGGCAAGTCCTTTGAGATGCTG
ATCCTGGGCCGCTTCATCATCGGTGTGTACTGCGGCCTGACCACA
GGCTTCGTGCCCATGTATGTGGGTGAAGTGTCACCCACAGCCCTT
CGTGGGGCCCTGGGCACCCTGCACCAGCTGGGCATCGTCGTCGGC
ATCCTCATCGCCCAGGTGTTCGGCCTGGACTCCATCATGGGCAAC
AAGGACCTGTGGCCCCTGCTGCTGAGCATCATCTTCATCCCGGCC
CTGCTGCAGTGCATCGTGCTGCCCTTCTGCCCCGAGAGTCCCCGC
TTCCTGCTCATCAACCGCAACGAGGAGAACCGGGCCAAGAGTGTG
CTAAAGAAGCTGCGCGGGACAGCTGACGTGACCCATGACCTGCAG
GAGATGAAGGAAGAGAGTCGGCAGATGATGCGGGAGAAGAAGGTC
ACCATCCTGGAGCTGTTCCGCTCCCCCGCCTACCGCCAGCCCATC
CTCATCGCTGTGGTGCTGCAGCTGTCCCAGCAGCTGTCTGGCATC
AACGCTGTCTTCTATTACTCCACGAGCATCTTCGAGAAGGCGGGG
GTGCAGCAGCCTGTGTATGCCACCATTGGCTCCGGTATCGTCAAC
ACGGCCTTCACTGTCGTGTCGCTGTTTGTGGTGGAGCGAGCAGGC
CGGCGGACCCTGCACCTCATAGGCCTCGCTGGCATGGCGGGTTGT
GCCATACTCATGACCATCGCGCTAGCACTGCTGGAGCAGCTACCC
TGGATGTCCTATCTGAGCATCGTGGCCATCTTTGGCTTTGTGGCC
TTCTTTGAAGTGGGTCCTGGCCCCATCCCATGGTTCATCGTGGCT
GAACTCTTCAGCCAGGGTCCACGTCCAGCTGCCATTGCCGTTGCA
GGCTTCTCCAACTGGACCTCAAATTTCATTGTGGGCATGTGCTTC
CAGTATGTGGAGCAACTGTGTGGTCCCTACGTCTTCATCATCTTC
ACTGTGCTCCTGGTTCTGTTCTTCATCTTCACCTACTTCTGAGGC
GCGCCCCCCTCTCCCTCCCCCCCCCCTAACGTTACTGGCCGAAGC
CGCTTGGAATAAGGCCGGTGTGCGTTTGTCTATATGTTATTTTCC
ACCATATTGCCGTCTTTTGGCAATGTGAGGGCCCGGAAACCTGGC
CCTGTCTTCTTGACGAGCATTCCTAGGGGTCTTTCCCCTCTCGCC
AAAGGAATGCAAGGTCTGTTGAATGTCGTGAAGGAAGCAGTTCCT
CTGGAAGCTTCTTGAAGACAAACAACGTCTGTAGCGACCCTTTGC
AGGCAGCGGAACCCCCCACCTGGCGACAGGTGCCTCTGCGGCCAA
AAGCCACGTGTATAAGATACACCTGCAAAGGCGGCACAACCCCAG
TGCCACGTTGTGAGTTGGATAGTTGTGGAAAGAGTCAAATGGCTC
TCCTCAAGCGTATTCAACAAGGGGCTGAAGGATGCCCAGAAGGTA
CCCCATTGTATGGGATCTGATCTGGGGCCTCGGTACACATGCTTT
ACATGTGTTTAGTCGAGGTTAAAAAAACGTCTAGGCCCCCCGAAC
CACGGGGACGTGGTTTTCCTTTGAAAAACACGATGATAATATGGC
CACAACCATGGCCAAGTTGACCAGTGCCGTTCCGGTGCTCACCGC
GCGCGACGTCGCCGGAGCGGTCGAGTTCTGGACCGACCGGCTCGG
GTTCTCCCGGGACTTCGTGGAGGACGACTTCGCCGGTGTGGTCCG
GGACGACGTGACCCTGTTCATCAGCGCGGTCCAGGACCAGGTGGT
GCCGGACAACACCCTGGCCTGGGTGTGGGTGCGCGGCCTGGACGA
GCTGTACGCCGAGTGGTCGGAGGTCGTGTCCACGAACTTCCGGGA
CGCCTCCGGGCCGGCCATGACCGAGATCGGCGAGCAGCCGTGGGG
GGGGAGTTCGCCCTGCGCGACCCGGCCGGCAACTGCGTGCACTTC
GTGGCCGAGGAGCAGGACTGA
Construct set 8: hLALBA-B4GALT1-GFP-
hGLUT1Δ-golgi (ST6)
Sequence 1 (SEQ ID NO: 26):
hLALBA(SEQ ID NO: 1)-GGATCCG(linker)-
IRES2(SEQ ID NO: 6)-hB4GALT1(SEQ ID
NO: 2)-CTCGAG(linker)-IRES2 (SEQ ID NO: 6)-
GS (SEQ ID NO: 7)
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGAGGATCCGCCCCTCTCCCTCCC
CCCCCCCTAACGTTACTGGCCGAAGCCGCTTGGAATAAGGCCGGT
GTGCGTTTGTCTATATGTTATTTTCCACCATATTGCCGTCTTTTG
GCAATGTGAGGGCCCGGAAACCTGGCCCTGTCTTCTTGACGAGCA
TTCCTAGGGGTCTTTCCCCTCTCGCCAAAGGAATGCAAGGTCTGT
TGAATGTCGTGAAGGAAGCAGTTCCTCTGGAAGCTTCTTGAAGAC
AAACAACGTCTGTAGCGACCCTTTGCAGGCAGCGGAACCCCCCAC
CTGGCGACAGGTGCCTCTGCGGCCAAAAGCCACGTGTATAAGATA
CACCTGCAAAGGCGGCACAACCCCAGTGCCACGTTGTGAGTTGGA
TAGTTGTGGAAAGAGTCAAATGGCTCTCCTCAAGCGTATTCAACA
AGGGGCTGAAGGATGCCCAGAAGGTACCCCATTGTATGGGATCTG
ATCTGGGGCCTCGGTACACATGCTTTACATGTGTTTAGTCGAGGT
TAAAAAAACGTCTAGGCCCCCCGAACCACGGGGACGTGGTTTTCC
TTTGAAAAACACGATGATAATATGGCCACAACCATGAGGCTTCGG
GAGCCGCTCCTGAGCGGCAGCGCCGCGATGCCAGGCGCGTCCCTA
CAGCGGGCCTGCCGCCTGCTCGTGGCCGTCTGCGCTCTGCACCTT
GGCGTCACCCTCGTTTACTACCTGGCTGGCCGCGACCTGAGCCGC
CTGCCCCAACTGGTCGGAGTCTCCACACCGCTGCAGGGCGGCTCG
AACAGTGCCGCCGCCATCGGGCAGTCCTCCGGGGAGCTCCGGACC
GGAGGGGCCCGGCCGCCGCCTCCTCTAGGCGCCTCCTCCCAGCCG
CGCCCGGGTGGCGACTCCAGCCCAGTCGTGGATTCTGGCCCTGGC
CCCGCTAGCAACTTGACCTCGGTCCCAGTGCCCCACACCACCGCA
CTGTCGCTGCCCGCCTGCCCTGAGGAGTCCCCGCTGCTTGTGGGC
CCCATGCTGATTGAGTTTAACATGCCTGTGGACCTGGAGCTCGTG
GCAAAGCAGAACCCAAATGTGAAGATGGGCGGCCGCTATGCCCCC
AGGGACTGCGTCTCTCCTCACAAGGTGGCCATCATCATTCCATTC
CGCAACCGGCAGGAGCACCTCAAGTACTGGCTATATTATTTGCAC
CCAGTCCTGCAGCGCCAGCAGCTGGACTATGGCATCTATGTTATC
AACCAGGCGGGAGACACTATATTCAATCGTGCTAAGCTCCTCAAT
GTTGGCTTTCAAGAAGCCTTGAAGGACTATGACTACACCTGCTTT
GTGTTTAGTGACGTGGACCTCATTCCAATGAATGACCATAATGCG
TACAGGTGTTTTTCACAGCCACGGCACATTTCCGTTGCAATGGAT
AAGTTTGGATTCAGCCTACCTTATGTTCAGTATTTTGGAGGTGTC
TCTGCTCTAAGTAAACAACAGTTTCTAACCATCAATGGATTTCCT
AATAATTATTGGGGCTGGGGAGGAGAAGATGATGACATTTTTAAC
AGATTAGTTTTTAGAGGCATGTCTATATCTCGCCCAAATGCTGTG
GTCGGGAGGTGTCGCATGATCCGCCACTCAAGAGACAAGAAAAAT
GAACCCAATCCTCAGAGGTTTGACCGAATTGCACACACAAAGGAG
ACAATGCTCTCTGATGGTTTGAACTCACTCACCTACCAGGTGCTG
GATGTACAGAGATACCCATTGTATACCCAAATCACAGTGGACATC
GGGACACCGAGCTAGCTCGAGCCCCTCTCCCTCCCCCCCCCCTAA
CGTTACTGGCCGAAGCCGCTTGGAATAAGGCCGGTGTGCGTTTGT
CTATATGTTATTTTCCACCATATTGCCGTCTTTTGGCAATGTGAG
GGCCCGGAAACCTGGCCCTGTCTTCTTGACGAGCATTCCTAGGGG
TCTTTCCCCTCTCGCCAAAGGAATGCAAGGTCTGTTGAATGTCGT
GAAGGAAGCAGTTCCTCTGGAAGCTTCTTGAAGACAAACAACGTC
TGTAGCGACCCTTTGCAGGCAGCGGAACCCCCCACCTGGCGACAG
GTGCCTCTGCGGCCAAAAGCCACGTGTATAAGATACACCTGCAAA
GGCGGCACAACCCCAGTGCCACGTTGTGAGTTGGATAGTTGTGGA
AAGAGTCAAATGGCTCTCCTCAAGCGTATTCAACAAGGGGCTGAA
GGATGCCCAGAAGGTACCCCATTGTATGGGATCTGATCTGGGGCC
TCGGTACACATGCTTTACATGTGTTTAGTCGAGGTTAAAAAAACG
TCTAGGCCCCCCGAACCACGGGGACGTGGTTTTCCTTTGAAAAAC
ACGATGATAATATGGCCACAACCATGGCCACCTCAGCAAGTTCCC
ACTTGAACAAAAACATCAAGCAAATGTACTTGTGCCTGCCCCAGG
GTGAGAAAGTCCAAGCCATGTATATCTGGGTTGATGGTACTGGAG
AAGGACTGCGCTGCAAAACCCGCACCCTGGACTGTGAGCCCAAGT
GTGTAGAAGAGTTACCTGAGTGGAATTTTGATGGCTCTAGTACCT
TTCAGTCTGAGGGCTCCAACAGTGACATGTATCTCAGCCCTGTTG
CCATGTTTCGGGACCCCTTCCGCAGAGATCCCAACAAGCTGGTGT
TCTGTGAAGTTTTCAAGTACAACCGGAAGCCTGCAGAGACCAATT
TAAGGCACTCGTGTAAACGGATAATGGACATGGTGAGCAACCAGC
ACCCCTGGTTTGGAATGGAACAGGAGTATACTCTGATGGGAACAG
ATGGGCACCCTTTTGGTTGGCCTTCCAATGGCTTTCCTGGGCCCC
AAGGTCCGTATTACTGTGGTGTGGGCGCAGACAAAGCCTATGGCA
GGGATATCGTGGAGGCTCACTACCGCGCCTGCTTGTATGCTGGGG
TCAAGATTACAGGAACAAATGCTGAGGTCATGCCTGCCCAGTGGG
AATTCCAAATAGGACCCTGTGAAGGAATCCGCATGGGAGATCATC
TCTGGGTGGCCCGTTTCATCTTGCATCGAGTATGTGAAGACTTTG
GGGTAATAGCAACCTTTGACCCCAAGCCCATTCCTGGGAACTGGA
ATGGTGCAGGCTGCCATACCAACTTTAGCACCAAGGCCATGCGGG
AGGAGAATGGTCTGAAGCACATCGAGGAGGCCATCGAGAAACTAA
GCAAGCGGCACCGGTACCACATTCGAGCCTACGATCCCAAGGGGG
GCCTGGACAATGCCCGTCGTCTGACTGGGTTCCACGAAACGTCCA
ACATCAACGACTTTTCTGCTGGTGTCGCCAATCGCAGTGCCAGCA
TCTGCATTCCCCGGACTGTCGGCCAGGAGAAGAAAGGTTACTTTG
AAGACCGCCGCCCCTCTGCCAATTGTGACCCCTTTGCAGTGACAG
AAGCCATCGTCCGCACATGCCTTCTCAATGAGACTGGCGACGAGC
CCTTCCAATACAAAAACTAA
Sequence 2 (SEQ ID NO: 27):
eGFP(SEQ ID NO: 5)-hGLUT1Δ(SEQ ID NO: 4)
(golgi(SEQ ID NO: 11))-TGAGGCGCGCC(linker)-
IRES2(SEQ ID NO: 6)-BleoR(SEQ ID NO: 8)
ATGGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGGACGGCAGCGTGCAGCTCGCC
GACCACTACCAGCAGAACACCCCCATCGGCGACGGCCCCGTGCTG
CTGCCCGACAACCACTACCTGAGCACCCAGTCCGCCCTGAGCAAA
GACCCCAACGAGAAGCGCGATCACATGGTCCTGCTGGAGTTCGTG
ACCGCCGCCGGGATCACTCTCGGCATGGACGAGCTGTACAAGGAG
CCCAGCAGCAAGAAGCTGACGGGTCGCCTCATGCTGGCCGTGGGA
GGAGCAGTGCTTGGCTCCCTGCAGTTTGGCTACAACACTGGAGTC
ATCAATGCCCCCCAGAAGGTGATCGAGGAGTTCTACAACCAGACA
TGGGTCCACCGCTATGGGGAGAGCATCCTGCCCACCACGCTCACC
ACGCTCTGGTCCCTCTCAGTGGCCATCTTTTCTGTTGGGGGCATG
ATTGGCTCCTTCTCTGTGGGCCTTTTCGTTAACCGCTTTGGCCGG
CGGAATTCAATGCTGATGATGAACCTGCTGGCCTTCGTGTCCGCC
GTGCTCATGGGCTTCTCGAAACTGGGCAAGTCCTTTGAGATGCTG
ATCCTGGGCCGCTTCATCATCGGTGTGTACTGCGGCCTGACCACA
GGCTTCGTGCCCATGTATGTGGGTGAAGTGTCACCCACAGCCCTT
CGTGGGGCCCTGGGCACCCTGCACCAGCTGGGCATCGTCGTCGGC
ATCCTCATCGCCCAGGTGTTCGGCCTGGACTCCATCATGGGCAAC
AAGGACCTGTGGCCCCTGCTGCTGAGCATCATCTTCATCCCGGCC
CTGCTGCAGTGCATCGTGCTGCCCTTCTGCCCCGAGAGTCCCCGC
TTCCTGCTCATCAACCGCAACGAGGAGAACCGGGCCAAGAGTGTG
CTAAAGAAGCTGCGCGGGACAGCTGACGTGACCCATGACCTGCAG
GAGATGAAGGAAGAGAGTCGGCAGATGATGCGGGAGAAGAAGGTC
ACCATCCTGGAGCTGTTCCGCTCCCCCGCCTACCGCCAGCCCATC
CTCATCGCTGTGGTGCTGCAGCTGTCCCAGCAGCTGTCTGGCATC
AACGCTGTCTTCTATTACTCCACGAGCATCTTCGAGAAGGCGGGG
GTGCAGCAGCCTGTGTATGCCACCATTGGCTCCGGTATCGTCAAC
ACGGCCTTCACTGTCGTGTCGCTGTTTGTGGTGGAGCGAGCAGGC
CGGCGGACCCTGCACCTCATAGGCCTCGCTGGCATGGCGGGTTGT
GCCATACTCATGACCATCGCGCTAGCACTGCTGGAGCAGCTACCC
TGGATGTCCTATCTGAGCATCGTGGCCATCTTTGGCTTTGTGGCC
TTCTTTGAAGTGGGTCCTGGCCCCATCCCATGGTTCATCGTGGCT
GAACTCTTCAGCCAGGGTCCACGTCCAGCTGCCATTGCCGTTGCA
GGCTTCTCCAACTGGACCTCAAATTTCATTGTGGGCATGTGCTTC
CAGTATGTGGAGCAACTGTGTGGTCCCTACGTCTTCATCATCTTC
ACTGTGCTCCTGGTTCTGTTCTTCATCTTCACCTACTTCCCCAGA
CAAGACACTACATCCATCCAACAAGGAGAAACAGCTTCAAAGGAG
AGAGTTATTGGTGTGTGAGGCGCGCCCCCCTCTCCCTCCCCCCCC
CCTAACGTTACTGGCCGAAGCCGCTTGGAATAAGGCCGGTGTGCG
TTTGTCTATATGTTATTTTCCACCATATTGCCGTCTTTTGGCAAT
GTGAGGGCCCGGAAACCTGGCCCTGTCTTCTTGACGAGCATTCCT
AGGGGTCTTTCCCCTCTCGCCAAAGGAATGCAAGGTCTGTTGAAT
GTCGTGAAGGAAGCAGTTCCTCTGGAAGCTTCTTGAAGACAAACA
ACGTCTGTAGCGACCCTTTGCAGGCAGCGGAACCCCCCACCTGGC
GACAGGTGCCTCTGCGGCCAAAAGCCACGTGTATAAGATACACCT
GCAAAGGCGGCACAACCCCAGTGCCACGTTGTGAGTTGGATAGTT
GTGGAAAGAGTCAAATGGCTCTCCTCAAGCGTATTCAACAAGGGG
CTGAAGGATGCCCAGAAGGTACCCCATTGTATGGGATCTGATCTG
GGGCCTCGGTACACATGCTTTACATGTGTTTAGTCGAGGTTAAAA
AAACGTCTAGGCCCCCCGAACCACGGGGACGTGGTTTTCCTTTGA
AAAACACGATGATAATATGGCCACAACCATGGCCAAGTTGACCAG
TGCCGTTCCGGTGCTCACCGCGCGCGACGTCGCCGGAGCGGTCGA
GTTCTGGACCGACCGGCTCGGGTTCTCCCGGGACTTCGTGGAGGA
CGACTTCGCCGGTGTGGTCCGGGACGACGTGACCCTGTTCATCAG
CGCGGTCCAGGACCAGGTGGTGCCGGACAACACCCTGGCCTGGGT
GTGGGTGCGCGGCCTGGACGAGCTGTACGCCGAGTGGTCGGAGGT
CGTGTCCACGAACTTCCGGGACGCCTCCGGGCCGGCCATGACCGA
GATCGGCGAGCAGCCGTGGGGGGGGAGTTCGCCCTGCGCGACCCG
GCCGGCAACTGCGTGCACTTCGTGGCCGAGGAGCAGGACTGA
Construct set 9: hLALBA-B4GALT1-GFP-
hGLUT1-golgi (ST4)
Sequence 1(SEQ ID NO: 28):
hLALBA (SEQ ID NO: 1)-GGATCCG(linker)-
IRES2(SEQ ID NO: 6)-B4GALT1 (SEQ ID NO: 2)-
CTCGAG(linker)-IRES2(SEQ ID NO: 6)-
GS(SEQ ID NO: 7)
ATGAGGTTCTTTGTCCCTCTGTTCCTGGTGGGCATCCTGTTCCCT
GCCATCCTGGCCAAGCAATTCACAAAATGTGAGCTGTCCCAGCTG
CTGAAAGACATAGATGGTTATGGAGGCATCGCTTTGCCTGAATTG
ATCTGTACCATGTTTCACACCAGTGGTTATGACACACAAGCCATA
GTTGAAAACAATGAAAGCACGGAATATGGACTCTTCCAGATCAGT
AATAAGCTTTGGTGCAAGAGCAGCCAGGTCCCTCAGTCAAGGAAC
ATCTGTGACATCTCCTGTGACAAGTTCCTGGATGATGACATTACT
GATGACATAATGTGTGCCAAGAAGATCCTGGATATTAAAGGAATT
GACTACTGGTTGGCCCATAAAGCCCTCTGCACTGAGAAGCTGGAA
CAGTGGCTTTGTGAGAAGTTGTGAGGATCCGCCCCTCTCCCTCCC
CCCCCCCTAACGTTACTGGCCGAAGCCGCTTGGAATAAGGCCGGT
GTGCGTTTGTCTATATGTTATTTTCCACCATATTGCCGTCTTTTG
GCAATGTGAGGGCCCGGAAACCTGGCCCTGTCTTCTTGACGAGCA
TTCCTAGGGGTCTTTCCCCTCTCGCCAAAGGAATGCAAGGTCTGT
TGAATGTCGTGAAGGAAGCAGTTCCTCTGGAAGCTTCTTGAAGAC
AAACAACGTCTGTAGCGACCCTTTGCAGGCAGCGGAACCCCCCAC
CTGGCGACAGGTGCCTCTGCGGCCAAAAGCCACGTGTATAAGATA
CACCTGCAAAGGCGGCACAACCCCAGTGCCACGTTGTGAGTTGGA
TAGTTGTGGAAAGAGTCAAATGGCTCTCCTCAAGCGTATTCAACA
AGGGGCTGAAGGATGCCCAGAAGGTACCCCATTGTATGGGATCTG
ATCTGGGGCCTCGGTACACATGCTTTACATGTGTTTAGTCGAGGT
TAAAAAAACGTCTAGGCCCCCCGAACCACGGGGACGTGGTTTTCC
TTTGAAAAACACGATGATAATATGGCCACAACCATGAGGCTTCGG
GAGCCGCTCCTGAGCGGCAGCGCCGCGATGCCAGGCGCGTCCCTA
CAGCGGGCCTGCCGCCTGCTCGTGGCCGTCTGCGCTCTGCACCTT
GGCGTCACCCTCGTTTACTACCTGGCTGGCCGCGACCTGAGCCGC
CTGCCCCAACTGGTCGGAGTCTCCACACCGCTGCAGGGCGGCTCG
AACAGTGCCGCCGCCATCGGGCAGTCCTCCGGGGAGCTCCGGACC
GGAGGGGCCCGGCCGCCGCCTCCTCTAGGCGCCTCCTCCCAGCCG
CGCCCGGGTGGCGACTCCAGCCCAGTCGTGGATTCTGGCCCTGGC
CCCGCTAGCAACTTGACCTCGGTCCCAGTGCCCCACACCACCGCA
CTGTCGCTGCCCGCCTGCCCTGAGGAGTCCCCGCTGCTTGTGGGC
CCCATGCTGATTGAGTTTAACATGCCTGTGGACCTGGAGCTCGTG
GCAAAGCAGAACCCAAATGTGAAGATGGGCGGCCGCTATGCCCCC
AGGGACTGCGTCTCTCCTCACAAGGTGGCCATCATCATTCCATTC
CGCAACCGGCAGGAGCACCTCAAGTACTGGCTATATTATTTGCAC
CCAGTCCTGCAGCGCCAGCAGCTGGACTATGGCATCTATGTTATC
AACCAGGCGGGAGACACTATATTCAATCGTGCTAAGCTCCTCAAT
GTTGGCTTTCAAGAAGCCTTGAAGGACTATGACTACACCTGCTTT
GTGTTTAGTGACGTGGACCTCATTCCAATGAATGACCATAATGCG
TACAGGTGTTTTTCACAGCCACGGCACATTTCCGTTGCAATGGAT
AAGTTTGGATTCAGCCTACCTTATGTTCAGTATTTTGGAGGTGTC
TCTGCTCTAAGTAAACAACAGTTTCTAACCATCAATGGATTTCCT
AATAATTATTGGGGCTGGGGAGGAGAAGATGATGACATTTTTAAC
AGATTAGTTTTTAGAGGCATGTCTATATCTCGCCCAAATGCTGTG
GTCGGGAGGTGTCGCATGATCCGCCACTCAAGAGACAAGAAAAAT
GAACCCAATCCTCAGAGGTTTGACCGAATTGCACACACAAAGGAG
ACAATGCTCTCTGATGGTTTGAACTCACTCACCTACCAGGTGCTG
GATGTACAGAGATACCCATTGTATACCCAAATCACAGTGGACATC
GGGACACCGAGCTAGctcgagCCCCTCTCCCTCCCCCCCCCCTAA
CGTTACTGGCCGAAGCCGCTTGGAATAAGGCCGGTGTGCGTTTGT
CTATATGTTATTTTCCACCATATTGCCGTCTTTTGGCAATGTGAG
GGCCCGGAAACCTGGCCCTGTCTTCTTGACGAGCATTCCTAGGGG
TCTTTCCCCTCTCGCCAAAGGAATGCAAGGTCTGTTGAATGTCGT
GAAGGAAGCAGTTCCTCTGGAAGCTTCTTGAAGACAAACAACGTC
TGTAGCGACCCTTTGCAGGCAGCGGAACCCCCCACCTGGCGACAG
GTGCCTCTGCGGCCAAAAGCCACGTGTATAAGATACACCTGCAAA
GGCGGCACAACCCCAGTGCCACGTTGTGAGTTGGATAGTTGTGGA
AAGAGTCAAATGGCTCTCCTCAAGCGTATTCAACAAGGGGCTGAA
GGATGCCCAGAAGGTACCCCATTGTATGGGATCTGATCTGGGGCC
TCGGTACACATGCTTTACATGTGTTTAGTCGAGGTTAAAAAAACG
TCTAGGCCCCCCGAACCACGGGGACGTGGTTTTCCTTTGAAAAAC
ACGATGATAATATGGCCACAACCATGGCCACCTCAGCAAGTTCCC
ACTTGAACAAAAACATCAAGCAAATGTACTTGTGCCTGCCCCAGG
GTGAGAAAGTCCAAGCCATGTATATCTGGGTTGATGGTACTGGAG
AAGGACTGCGCTGCAAAACCCGCACCCTGGACTGTGAGCCCAAGT
GTGTAGAAGAGTTACCTGAGTGGAATTTTGATGGCTCTAGTACCT
TTCAGTCTGAGGGCTCCAACAGTGACATGTATCTCAGCCCTGTTG
CCATGTTTCGGGACCCCTTCCGCAGAGATCCCAACAAGCTGGTGT
TCTGTGAAGTTTTCAAGTACAACCGGAAGCCTGCAGAGACCAATT
TAAGGCACTCGTGTAAACGGATAATGGACATGGTGAGCAACCAGC
ACCCCTGGTTTGGAATGGAACAGGAGTATACTCTGATGGGAACAG
ATGGGCACCCTTTTGGTTGGCCTTCCAATGGCTTTCCTGGGCCCC
AAGGTCCGTATTACTGTGGTGTGGGCGCAGACAAAGCCTATGGCA
GGGATATCGTGGAGGCTCACTACCGCGCCTGCTTGTATGCTGGGG
TCAAGATTACAGGAACAAATGCTGAGGTCATGCCTGCCCAGTGGG
AATTCCAAATAGGACCCTGTGAAGGAATCCGCATGGGAGATCATC
TCTGGGTGGCCCGTTTCATCTTGCATCGAGTATGTGAAGACTTTG
GGGTAATAGCAACCTTTGACCCCAAGCCCATTCCTGGGAACTGGA
ATGGTGCAGGCTGCCATACCAACTTTAGCACCAAGGCCATGCGGG
AGGAGAATGGTCTGAAGCACATCGAGGAGGCCATCGAGAAACTAA
GCAAGCGGCACCGGTACCACATTCGAGCCTACGATCCCAAGGGGG
GCCTGGACAATGCCCGTCGTCTGACTGGGTTCCACGAAACGTCCA
ACATCAACGACTTTTCTGCTGGTGTCGCCAATCGCAGTGCCAGCA
TCTGCATTCCCCGGACTGTCGGCCAGGAGAAGAAAGGTTACTTTG
AAGACCGCCGCCCCTCTGCCAATTGTGACCCCTTTGCAGTGACAG
AAGCCATCGTCCGCACATGCCTTCTCAATGAGACTGGCGACGAGC
CCTTCCAATACAAAAACTAA
Sequence 2 (SEQ ID NO: 29):
eGFP (SEQ ID NO: 5)-hGLUT1(SEQ ID NO: 3)
(golgi (SEQ ID NO: 11))-TGAGGCGCGCC(linker)-
IRES2(SEQ ID NO: 6)-BleoR(SEQ ID NO: 8)
ATGGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGGACGGCAGCGTGCAGCTCGCC
GACCACTACCAGCAGAACACCCCCATCGGCGACGGCCCCGTGCTG
CTGCCCGACAACCACTACCTGAGCACCCAGTCCGCCCTGAGCAAA
GACCCCAACGAGAAGCGCGATCACATGGTCCTGCTGGAGTTCGTG
ACCGCCGCCGGGATCACTCTCGGCATGGACGAGCTGTACAAGGAG
CCCAGCAGCAAGAAGCTGACGGGTCGCCTCATGCTGGCCGTGGGA
GGAGCAGTGCTTGGCTCCCTGCAGTTTGGCTACAACACTGGAGTC
ATCAATGCCCCCCAGAAGGTGATCGAGGAGTTCTACAACCAGACA
TGGGTCCACCGCTATGGGGAGAGCATCCTGCCCACCACGCTCACC
ACGCTCTGGTCCCTCTCAGTGGCCATCTTTTCTGTTGGGGGCATG
ATTGGCTCCTTCTCTGTGGGCCTTTTCGTTAACCGCTTTGGCCGG
CGGAATTCAATGCTGATGATGAACCTGCTGGCCTTCGTGTCCGCC
GTGCTCATGGGCTTCTCGAAACTGGGCAAGTCCTTTGAGATGCTG
ATCCTGGGCCGCTTCATCATCGGTGTGTACTGCGGCCTGACCACA
GGCTTCGTGCCCATGTATGTGGGTGAAGTGTCACCCACAGCCCTT
CGTGGGGCCCTGGGCACCCTGCACCAGCTGGGCATCGTCGTCGGC
ATCCTCATCGCCCAGGTGTTCGGCCTGGACTCCATCATGGGCAAC
AAGGACCTGTGGCCCCTGCTGCTGAGCATCATCTTCATCCCGGCC
CTGCTGCAGTGCATCGTGCTGCCCTTCTGCCCCGAGAGTCCCCGC
TTCCTGCTCATCAACCGCAACGAGGAGAACCGGGCCAAGAGTGTG
CTAAAGAAGCTGCGCGGGACAGCTGACGTGACCCATGACCTGCAG
GAGATGAAGGAAGAGAGTCGGCAGATGATGCGGGAGAAGAAGGTC
ACCATCCTGGAGCTGTTCCGCTCCCCCGCCTACCGCCAGCCCATC
CTCATCGCTGTGGTGCTGCAGCTGTCCCAGCAGCTGTCTGGCATC
AACGCTGTCTTCTATTACTCCACGAGCATCTTCGAGAAGGCGGGG
GTGCAGCAGCCTGTGTATGCCACCATTGGCTCCGGTATCGTCAAC
ACGGCCTTCACTGTCGTGTCGCTGTTTGTGGTGGAGCGAGCAGGC
CGGCGGACCCTGCACCTCATAGGCCTCGCTGGCATGGCGGGTTGT
GCCATACTCATGACCATCGCGCTAGCACTGCTGGAGCAGCTACCC
TGGATGTCCTATCTGAGCATCGTGGCCATCTTTGGCTTTGTGGCC
TTCTTTGAAGTGGGTCCTGGCCCCATCCCATGGTTCATCGTGGCT
GAACTCTTCAGCCAGGGTCCACGTCCAGCTGCCATTGCCGTTGCA
GGCTTCTCCAACTGGACCTCAAATTTCATTGTGGGCATGTGCTTC
CAGTATGTGGAGCAACTGTGTGGTCCCTACGTCTTCATCATCTTC
ACTGTGCTCCTGGTTCTGTTCTTCATCTTCACCTACTTCAAAGTT
CCTGAGACTAAAGGCCGGACCTTCGATGAGATCGCTTCCGGCTTC
CGGCAGGGGGGAGCCAGCCAAAGTGACAAGACACCCGAGGAGCTG
TTCCATCCCCTGGGGGCTGATTCCCAAGTGCCCAGACAAGACACT
ACATCCATCCAACAAGGAGAAACAGCTTCAAAGGAGAGAGTTATT
GGTGTGTGAGGCGCGCCCCCCTCTCCCTCCCCCCCCCCTAACGTT
ACTGGCCGAAGCCGCTTGGAATAAGGCCGGTGTGCGTTTGTCTAT
ATGTTATTTTCCACCATATTGCCGTCTTTTGGCAATGTGAGGGCC
CGGAAACCTGGCCCTGTCTTCTTGACGAGCATTCCTAGGGGTCTT
TCCCCTCTCGCCAAAGGAATGCAAGGTCTGTTGAATGTCGTGAAG
GAAGCAGTTCCTCTGGAAGCTTCTTGAAGACAAACAACGTCTGTA
GCGACCCTTTGCAGGCAGCGGAACCCCCCACCTGGCGACAGGTGC
CTCTGCGGCCAAAAGCCACGTGTATAAGATACACCTGCAAAGGCG
GCACAACCCCAGTGCCACGTTGTGAGTTGGATAGTTGTGGAAAGA
GTCAAATGGCTCTCCTCAAGCGTATTCAACAAGGGGCTGAAGGAT
GCCCAGAAGGTACCCCATTGTATGGGATCTGATCTGGGGCCTCGG
TACACATGCTTTACATGTGTTTAGTCGAGGTTAAAAAAACGTCTA
GGCCCCCCGAACCACGGGGACGTGGTTTTCCTTTGAAAAACACGA
TGATAATATGGCCACAACCATGGCCAAGTTGACCAGTGCCGTTCC
GGTGCTCACCGCGCGCGACGTCGCCGGAGCGGTCGAGTTCTGGAC
CGACCGGCTCGGGTTCTCCCGGGACTTCGTGGAGGACGACTTCGC
CGGTGTGGTCCGGGACGACGTGACCCTGTTCATCAGCGCGGTCCA
GGACCAGGTGGTGCCGGACAACACCCTGGCCTGGGTGTGGGTGCG
CGGCCTGGACGAGCTGTACGCCGAGTGGTCGGAGGTCGTGTCCAC
GAACTTCCGGGACGCCTCCGGGCCGGCCATGACCGAGATCGGCGA
GCAGCCGTGGGGGCGGGAGTTCGCCCTGCGCGACCCGGCCGGCAA
CTGCGTGCACTTCGTGGCCGAGGAGCAGGACTGA
ER localization sequence is LLTKVKGS
(SEQ ID NO: 30)
Golgi localization sequence is
PRQDTTSIQQGETASKERVIGV (SEQ ID NO: 31)
or
TTSIQQGETASKERVIGV
(SEQ ID NO: 32; CST C-terminal 18 amino acids)
CST C-terminal Golgi localisation sequence
(nucleic acid):
ACTACATCCATCCAACAAGGAGAAACAGCTTCAAAGGAGAGAGTT
ATTGGTGTG
(SEQ ID NO: 33)

1. A recombinant cell for producing lactose, wherein said recombinant cell comprises one or more expression constructs that encode an alpha-lactalbumin (LALBA) and a beta-1,4-galactosyltransferase 1 (B4GalT1).

2. One or more expression constructs, wherein the expression constructs comprise polynucleotides encoding an alpha-lactalbumin (LALBA) and a beta-1,4-galactosyltransferase 1 (B4GalT1).

3. The expression constructs of claim 2, wherein the one or more expression constructs encode an additional glucose transporter.

4. The expression constructs of claim 3, wherein the glucose transporter is selected from the group consisting of a glucose transporter 1 (GLUT1), a glucose transporter 8 (GLUT8), a glucose transporter 12 (GLUT12), and a sodium-glucose transporter (SGLT1).

5. The expression constructs of claim 3, wherein the glucose transporter is a glucose transporter 1 (GLUT1) or wherein the glucose transporter is a human glucose transporter 1 (huGLUT1).

6.-7. (canceled)

8. The expression constructs of claim 3, wherein the glucose transporter is a full-length GLUT1 or C-terminal truncated GLUT1.

9. The expression constructs of claim 2, wherein the alpha-lactalbumin (LALBA) and the beta-1,4-galactosyltransferase 1 (B4GalT1) are from the same mammalian species.

10. The recombinant cell or expression constructs of claim 2, wherein both the alpha-lactalbumin (LALBA) and the beta-1,4-galactosyltransferase 1 (B4GalT1) are from human.

11. The expression constructs of claim 2, wherein both the alpha-lactalbumin (LALBA) and the beta-1,4-galactosyltransferase 1 (B4GalT1) are from hamster.

12. The expression constructs of claim 2, wherein the one or more expression constructs are mammalian expression vector.

13. The recombinant cell of claim 1, wherein the cell is of mammalian origin.

14. The expression constructs of claim 3, wherein the sequences encoding alpha-lactalbumin (LALBA), beta-1,4-galactosyltransferase 1 (B4GalT1), glucose transporter, and combinations thereof, are engineered in at least one, at least two, at least three or at least four constructs.

15. The recombinant cell of claim 1, wherein the recombinant cell comprises:
an expression construct that encodes an alpha-lactalbumin (LALBA), a beta-1,4-galactosyltransferase 1 (B4GalT1), a glucose transporter 1 (GLUT1), and a marker protein,
wherein C-terminus of LALBA is linked to N-terminus of B4GalT1, C-terminus of B4GalT1 is linked to N-terminus of GLUT1, and C-terminus of GLUT1 is fused to N-terminus of the marker protein
and an expression construct that encodes a beta-1,4-galactosyltransferase 1 (B4GalT1) and an alpha-lactalbumin (LALBA),
wherein C-terminus of B4GalT1 is linked to N-terminus of LALBA;
or
an expression construct that encodes an alpha-lactalbumin (LALBA), a beta-1,4-galactosyltransferase 1 (B4GalT1), a marker protein, a glucose transporter 1 (huGLUT1), and an ER localization signal,
wherein C-terminus of LALBA is linked to N-terminus of B4GalT1, C-terminus of B4GalT1 is linked to N-terminus of the marker protein, C-terminus of the marker protein is linked to N-terminus of GLUT1, and C-terminus of GLUT1 is linked to N-terminus of the ER localization signal
and an expression construct that encodes a beta-1,4-galactosyltransferase 1 (B4GalT1) and an alpha-lactalbumin (LALBA),
wherein C-terminus of B4GalT1 is linked to N-terminus of LALBA;
or
an expression construct that encodes an alpha-lactalbumin (LALBA), a beta-1,4-galactosyltransferase 1 (B4GalT1), a marker protein, and a glucose transporter 1 (GLUT1),
wherein C-terminus of LALBA is linked to N-terminus of B4GalT1, C-terminus of B4GalT1 is linked to N-terminus of the marker protein, and C-terminus of the marker protein is linked to N-terminus of GLUT1
and an expression construct that encodes a beta-1,4-galactosyltransferase 1 (B4GalT1) and an alpha-lactalbumin (LALBA),
wherein C-terminus of B4GalT1 is linked to N-terminus of LALBA;
or
a first expression construct that encodes an alpha-lactalbumin (LALBA) and a second expression construct that encodes a beta-1,4-galactosyltransferase 1 (B4GalT1);
or
an expression construct that encodes an alpha-lactalbumin (LALBA), a beta-1,4-galactosyltransferase 1 (B4GalT1), a marker protein, and a C-terminal truncated glucose transporter 1 (GLUT1Δ),
wherein C-terminus of LALBA is linked to N-terminus of B4GalT1, C-terminus of B4GalT1 is linked to N-terminus of the marker protein, and C-terminus of the marker protein is linked to N-terminus of GLUT1Δ
and an expression construct that encodes a beta-1,4-galactosyltransferase 1 (B4GalT1) and an alpha-lactalbumin (LALBA),
wherein C-terminus of B4GalT1 is linked to N-terminus of LALBA;
or
an expression construct that encodes an alpha-lactalbumin (LALBA), a beta-1,4-galactosyltransferase 1 (B4GalT1), a marker protein, a C-terminal truncated glucose transporter 1 (GLUT14), and a Golgi localization sequence,
wherein C-terminus of LALBA is linked to N-terminus of B4GalT1, C-terminus of B4GalT1 is linked to N-terminus of the marker protein, C-terminus of the marker protein is linked to N-terminus of GLUT1Δ, and C-terminus of GLUT1Δ is linked to N-terminus of the Golgi localization sequence and an expression construct that encodes a beta-1,4-galactosyltransferase 1 (B4GalT1) and an alpha-lactalbumin (LALBA),
wherein C-terminus of B4GalT1 is linked to N-terminus of LALBA;
or
an expression construct that encodes an alpha-lactalbumin (LALBA), a beta-1,4-galactosyltransferase 1 (B4GalT1), a marker protein, a glucose transporter 1 (huGLUT1), and a Golgi localization sequence,
wherein C-terminus of LALBA is linked to N-terminus of B4GalT1, C-terminus of B4GalT1 is linked to N-terminus of the marker protein, C-terminus of the marker protein is linked to N-terminus of GLUT1, and C-terminus of GLUT1 is linked to N-terminus of C-terminal of the Golgi localization sequence
and an expression construct that encodes a beta-1,4-galactosyltransferase 1 (B4GalT1) linked to alpha-lactalbumin (LALBA)
wherein C-terminus of B4GalT1 is linked to N-terminus of LALBA;
or
an expression construct that encodes an alpha-lactalbumin (LALBA), a beta-1,4-galactosyltransferase 1 (B4GalT1), a marker protein, and a glucose transporter 1 (GLUT1),
wherein C-terminus of LALBA is linked to N-terminus of B4GalT1, C-terminus of B4GalT1 is linked to N-terminus of the marker protein, and C-terminus of the marker protein is linked to N-terminus of GLUT1.

16. The recombinant cell of claim 1, wherein the expression constructs comprise, if present, the following:
sequences having at least 90% or 95% or 98% or 100% sequence identity with SEQ ID NO: 1 (alpha-lactalbumin (LALBA));
sequences having at least 90% or 95% or 98% or 100% sequence identity with SEQ ID NO: 2 (beta-1,4-galactosyltransferase 1 (B4GalT1)); and
sequences having at least 90% or 95% or 98% or 100% sequence identity with SEQ ID NO: 3 (glucose transporter 1 (GLUT1)); or
sequences having at least 90% or 95% or 98% or 100% sequence identity with SEQ ID NO: 4 (C-terminal truncated glucose transporter 1 (GLUT1Δ)).

17. The expression constructs of claim 2, comprising the following:
polynucleotide sequences having at least 90% or 95% or 98% or 100% sequence identity with SEQ ID NO: 1 (alpha-lactalbumin (LALBA));
polynucleotide sequences having at least 90% or 95% or 98% or 100% sequence identity with SEQ ID NO: 2 (beta-1,4-galactosyltransferase 1 (B4GalT1)); and optionally
polynucleotide sequences having at least 90% or 95% or 98% or 100% sequence identity with SEQ ID NO: 3 (glucose transporter 1 (GLUT1)); or
polynucleotide sequences having at least 90% or 95% or 98% or 100% sequence identity with SEQ ID NO: 4 (C-terminal truncated glucose transporter 1 (GLUT1Δ)).

18. A method of producing lactose using the recombinant cell of claim 1, wherein the method comprises the steps of:
i) culturing the recombinant cell of any one of the preceding claims, and
ii) detecting lactose from the recombinant cell culture media in i).

19.-20. (canceled)

21. The recombinant cell of claim 1, wherein the lactose is further modified within said cell to produce human milk oligosaccharides (HMOs).

22. The recombinant cell of claim 21, wherein the human milk oligosaccharides (HMOs) produced are selected from the group consisting of sialyl-lactose, lacto-N-neotetraose (LNnT), sialyl-LNnT, para-lacto-N-hexaose (para-LNH), sialyl-para-LNH, para-lacto-N-octaose.

23. The recombinant cell of claim 22, wherein the sialyl-lactose is a 3′ sialyl-lactose (3′SL).

24. The one or more expression constructs of claim 2, wherein the one or more expression constructs are comprised in a recombinant cell.
